(19) United States
(12) Patent Application Publication
(10) Pub. No.: US 2007/0032537 A1
(43) Pub. Date: Feb. 8, 2007

(54) 5-Substituted 2h-pyrazole-3-carboxylic acid derivatives as agonists for the acid receptor rup25 for the treatment of dyslipidemia and related diseases

(75) Inventors: Graeme Semple, San Diego, CA (US); Tawfik Gharbaoui, San Diego, CA (US); Young-Jun Shin, San Diego, CA (US); Marc Decaire, San Diego, CA (US); Claudia L. Averbuj, San Diego, CA (US); Philip J. Skinner, San Diego, CA (US)

Correspondence Address: ARENA PHARMACEUTICALS, INC., 6166 NANCY RIDGE DRIVE, SAN DIEGO, CA, 92121, US

(73) Assignee: Arena Pharmaceuticals, Inc., San Diego, CA (US)

(21) Appl. No.: 10/560,332

(22) PCT Filed: Jun. 10, 2004
(86) PCT No.: PCT/US04/18389

§ 371 Date: Sep. 8, 2006

Related U.S. Application Data
(60) Provisional application No. 60/478,664, filed on Jun. 13, 2003.

(51) Int. Cl. A61K 31/4152; C07D 231/02

(52) U.S. Cl. 514/406; 548/374.1

(57) ABSTRACT

The present invention relates to certain pyrazole carboxylic acid and ester derivatives, and pharmaceutically acceptable salts thereof, which exhibit useful pharmaceutical properties, for example as agonists for the RUP25 receptor. (I) Also provided by the present invention are pharmaceutical compositions containing compounds of the invention, and methods of using the compounds and compositions of the invention in the prophylaxis or treatment of metabolic-related disorders, including dyslipidemia, atherosclerosis, coronary heart disease, insulin resistance, type 2 diabetes, Syndrome-X and the like. In addition, the present invention also provides for the use of the compounds of the invention in combination with other active agents such as those belonging to the class of α-glucosidase inhibitors, aldose reductase inhibitors, biguanides, HMG-CoA reductase inhibitors, squalene synthesis inhibitors, fibrates, LDL catabolism enhancers, angiotensin converting enzyme (ACE) inhibitors, insulin secretion enhancers and the like the substituents are defined in claim 1.

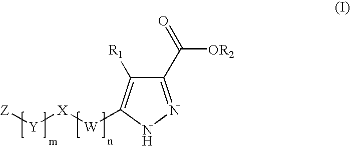

### FIELD OF THE INVENTION

The present invention relates to certain pyrazole carboxylic acid and ester derivatives, and pharmaceutically acceptable salts thereof, which exhibit useful pharmaceutical properties, for example as agonists for the nicotinic acid receptor, refered to as RUP25 herein. Also provided by the present invention are pharmaceutical compositions containing one or more compounds of the invention, and methods of using the compounds and compositions of the invention in the prophylaxis or treatment of metabolic-related disorders, including dyslipidemia, atherosclerosis, coronary heart disease, insulin resistance, type 2 diabetes, Syndrome-X and the like. In addition, the present invention also provides for the use of the compounds of the invention in combination with other active agents such as those belonging to the class of α-glucosidase inhibitors, aldose reductase inhibitors, biguanides, HMG-CoA reductase inhibitors, squalene synthesis inhibitors, fibrates, LDL catabolism enhancers, angiotensin converting enzyme (ACE) inhibitors, insulin secretion enhancers, thiazolidinedione and the like.

### BACKGROUND OF THE INVENTION

Compounds of the Invention as Antilipolytic Agents

Atherosclerosis and stroke are the numbers one and number three leading causes of death of both men and women in the United States. Type 2 diabetes is a public health problem that is serious, widespread and increasing. Elevated levels of low density lipoprotein (LDL) cholesterol or low levels of high density lipoprotein (HDL) cholesterol are, independently, risk factors for atherosclerosis and associated cardiovascular pathologies. In addition, high levels of plasma free fatty acids are associated with insulin resistance and type 2 diabetes. One strategy for decreasing LDL-cholesterol, increasing HDL-cholesterol, and decreasing plasma free fatty acids is to inhibit lipolysis in adipose tissue. This approach involves regulation of hormone sensitive lipase, which is the rate-limiting enzyme in lipolysis. Lipolytic agents increase cellular levels of cAMP, which leads to activation of hormone sensitive lipase within adipocytes. Agents that lower intracellular cAMP levels, by contrast, would be antilipolytic.

It is also worth noting in passing that an increase in cellular levels of cAMP down-regulates the secretion of adiponectin from adipocytes [Delporte, M L et al. Biochem J (2002) July]. Reduced levels of plasma adiponectin have been associated with metabolic-related disorders, including atherosclerosis, coronary heart disease, insulin resistance and type 2 diabetes [Matsuda, M et al. J Biol Chem (2002) July and reviewed therein].

Nicotinic acid (niacin, pyridine-3-carboxylic acid) is a water-soluble vitamin required by the human body for health, growth and reproduction; a part of the Vitamin B complex. Nicotinic acid is also one of the oldest used drugs for the treatment of dyslipidemia. It is a valuable drug in that it favorably affects virtually all of the lipid parameters listed above [Goodman and Gilman's Pharmacological Basis of Therapeutics, editors Harmon J G and Limbird L E, Chapter 36, Mahley R W and Bersot T P (2001) pages 971-1002]. The benefits of nicotinic acid in the treatment or prevention of atherosclerotic cardiovascular disease have been documented in six major clinical trials [Guyton J R (1998) Am J Cardiol 82:18U-23U]. Nicotinic acid and related derivatives, such as, acipimox have recently been discussed [Lorenzen, A et al (2001) Molecular Pharmacology 59:349-357].

Nicotinic acid and currently existing analogs thereof inhibit the production and release of free fatty acids from adipose tissue, likely via an inhibition of adenylyl cyclase, a decrease in intracellular cAMP levels, and a concomitant decrease in hormone sensitive lipase activity. Agonists that down-regulate hormone sensitive lipase activity leading to a decrease in plasma free fatty acid levels are likely to have therapeutic value. The consequence of decreasing plasma free fatty acids is two-fold. First, it will ultimately lower LDL-cholesterol and raise HDL-cholesterol levels, independent risk factors, thereby reducing the risk of mortality due to cardiovascular incidence subsequent to atheroma formation. Second, it will provide an increase in insulin sensitivity in individuals with insulin resistance or type 2 diabetes.

The rational development of novel, nicotinic acid receptor agonists that have fewer side-effects will be valuable, but to date this has been hindered by the inability to molecularly identify the nicotinic acid receptor. Furthermore, other receptors of the same class may exist on the surface of adipocytes and similarly decrease hormone sensitive lipase activity through a reduction in the level of intracellular cAMP but without the elicitation of adverse effects such as flushing, thereby representing promising novel therapeutic targets. Recent work suggests that nicotinic acid probably acts through a specific GPCR [Lorenzen A, et al. (2001) Molecular Pharmacology 59:349-357 and reviewed therein]. Further work has suggested that the effects of nicotinic acid on macrophages, spleen and probably adipocytes are mediated via this specific GPCR [Lorenzen A, et al. (2002) Biochemical Pharmacology 64:645-648 and reviewed therein].

Unfortunately, the use of nicotinic acid as a therapeutic agent is partially limited by a number of associated, adverse side-effects. These include flushing, free fatty acid rebound, and liver toxicity. The most noticeable side-effect associated with nicotinic acid is flushing. An individual may develop a visible, uncomfortable, hot or flushed feeling following each dose. Accordingly, there is a need for compounds and compositions with improved therapeutic activity with minimal side effects.

This application is related to U.S. Provisional Patent Application, Ser. No. 60/478,664, incorporated herein by reference in its entirety.

### SUMMARY OF THE INVENTION

The present invention is drawn to compounds which bind to and modulate the activity of a GPCR referred to herein as RUP25, and uses thereof. The term RUP25 as used herein includes the human sequences found in GeneBank accession number NP_{—}808219, naturally-occurring allelic variants, mammalian orthologs, and recombinant mutants thereof.

One aspect of the present invention encompasses pyrazole carboxylic acid and ester derivatives as shown in Formula (I):

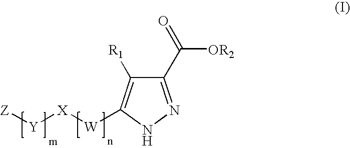

wherein:

W and Y are independently a straight or branched chain C₁₋₅ alkylene group optionally containing one double bond, one triple bond or carbonyl, wherein said C₁₋₅ alkylene group is optionally substituted with halogen, hydroxyl, C₁₋₄ alkyl, C₁₋₄ haloalkyl or C₁₋₄ alkoxy;

X is —NR₃C(O)—, —C(O)NR₃, —NR₃S(O)₂—, —S(O)₂NR₃—, —NR₃C(O)NR₄—, —NR₃C(O)O—, —OC(O)NR₃—, —NR₃—, —C(O)—, —CH(OH)—, —C(NH)—, —O—, —S—, —S(O)— or —S(O)₂—;

R₃ and R4 are independently H, C₁₋₄ alkyl, phenyl or heteroaryl, wherein each of the alkyl, phenyl and heteroaryl are optionally substituted with 1 to 5 substituents selected from the group consisting of halogen, hydroxyl, thiol, cyano, nitro, C₁₋₄ haloalkyl, amino, C₁₋₄ alkylamino, di-C₁₋₄-alkylamino, C₁₋₄ alkyl, C₁₋₄ alkoxy, C₂₋₄ alkenyl, C₂₋₄ alkynyl, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl;

Z is H, halogen, phenyl or heteroaryl, wherein said phenyl and heteroaryl are optionally substituted with 1 to 5 substituents selected from the group consisting of halogen, hydroxy, thiol, cyano, nitro, C₁₋₄ haloalkyl, amino, C₁₋₄ alkylamino, di-C₁₋₄-alkylamino, C₁₋₄ alkyl, C₁₋₄ alkoxy, C₂₋₄ alkenyl, C₂₋₄ alkynyl, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl;

R₁ is H, hydroxyl, halogen, C₁₋₄ alkyl or C₁₋₄ haloalkyl;

R₂ is H or C₁₋₈ alkyl and

“n” and “m” are each independently 0 or 1; or

a pharmaceutically acceptable salt, solvate or hydrate thereof;

provided that when X is —NR₃— then “n” is 1.

One aspect of the present invention encompasses pharmaceutical compositions comprising at least one compound according to Formula (I), as described herein. In some embodiments, the pharmaceutical composition further comprises one or more agents selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

One embodiment of the present invention pertains to pharmaceutical compositions comprising a α-glucosidase inhibitor. In some embodiments the α-glucosidase inhibitor is acarbose, voglibose or miglitol. In some embodiments the α-glucosidase inhibitor is voglibose.

One embodiment of the present invention pertains to pharmaceutical compositions comprising an aldose reductase inhibitor. In some embodiments the aldose reductase inhibitor is tolurestat; epalrestat; imirestat; zenarestat; zopolrestat; or sorbinil.

One embodiment of the present invention pertains to pharmaceutical compositions comprising a biguanide. In some embodiments the biguanide is phenformin, metformin or buformin. In some embodiments the biguanide is metformin.

One embodiment of the present invention pertains to pharmaceutical compositions comprising a HMG-CoA reductase inhibitor. In some embodiments the HMG-CoA reductase inhibitor is rosuvastatin, pravastatin, simvastatin, lovastatin, atorvastatin, fluvastatin or cerivastatin.

One embodiment of the present invention pertains to pharmaceutical compositions comprising a fibrate. In some embodiments the fibrate is bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, or theofibrate.

One embodiment of the present invention pertains to pharmaceutical compositions comprising an angiotensin converting enzyme inhibitor. In some embodiments the angiotensin converting enzyme inhibitor is captopril, enalapril, alacepril, delapril; ramipril, lisinopril, imidapril, benazepril, ceronapril, cilazapril, enalaprilat, fosinopril, moveltopril, perindopril, quinapril, spirapril, temocapril or trandolapril.

One embodiment of the present invention pertains to pharmaceutical compositions comprising an insulin secretion enhancer. In some embodiments the insulin secretion enhancer is tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolcyclamide, glimepiride, nateglinide, or mitiglinide.

One embodiment of the present invention pertains to pharmaceutical compositions comprising a thiazolidinedione. In some embodiments the thiazolidinedione is rosiglitazone or pioglitazone. In some embodiments the thiazolidinedione is rosiglitazone.

One aspect of the present invention pertains to a compound of Formula (I), as described herein, for use in a method of treatment of the human or animal body by therapy.

One aspect of the present invention pertains to a compound of Formula (I), as described herein, for use in a method of prophylaxis or treatment of a metabolic-related disorder of the human or animal body by therapy.

One aspect of the present invention pertains to methods for prophylaxis or treatment of a metabolic-related disorder in an individual in need of prophylaxis or treatment comprising administering to the individual a therapeutically effective amount of a compound according of Formula (I), as described, or a pharmaceutical composition.

One aspect of the present invention pertains to methods of modulating a RUP25 receptor in an individual comprising contacting the receptor with a compound according Formula (I). In some embodiments the compound is an agonist In some embodiments the modulation of the RUP25 receptor is for prophylaxis or treatment of a metabolic-related disorder in an individual in need of said prophylaxis or treatment.

One embodiment of the present invention relates to methods of prophylaxis or treatment of metabolic-related disorders. In some embodiments the metabolic-related disorder is of the group consisting of dyslipidemia, atherosclerosis, coronary heart disease, insulin resistance, obesity, impaired glucose tolerance, atheromatous disease, hypertension, stroke, Syndrome X, heart disease and type 2 diabetes. In some embodiments the metabolic-related disorder is dyslipidemia, atherosclerosis, coronary heart disease, insulin resistance and type 2 diabetes. In some embodiments the metabolic-related disorder is dyslipidemia. In some embodiments the metabolic-related disorder is atherosclerosis. In some embodiments the metabolic-related disorder is coronary heart disease. In some embodiments the metabolic-related disorder is insulin resistance. In some embodiments the metabolic-related disorder is type 2 diabetes.

One aspect of the present invention encompasses compounds of Formula (I) for production of a medicament for use in prophylaxis or treatment of a metabolic-related disorder. In some embodiments, the use of a compound of Formula (I) for production of a medicament further comprises one or more agents selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione. In some embodiments the agent is a α-glucosidase inhibitor. In some embodiments the α-glucosidase inhibitor is acarbose, voglibose or miglitol. In some embodiments the α-glucosidase inhibitor is voglibose. In some embodiments the agent is an aldose reductase inhibitor. In some embodiments the aldose reductase inhibitor is tolurestat; epalrestat; imirestat; zenarestat; zopolrestat; or sorbinil. In some embodiments the agent is a biguanide. In some embodiments the biguanide is phenformin, metformin or buformin. In some embodiments the biguanide is metformin. In some embodiments the agent is a HMG-CoA reductase inhibitor. In some embodiments the HMG-CoA reductase inhibitor is rosuvastatin, pravastatin, simvastatin, lovastatin, atorvastatin, fluvastatin or cerivastatin. In some embodiments the agent is a fibrate. In some embodiments the fibrate is bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, or theofibrate. In some embodiments the agent is an angiotensin converting enzyme inhibitor. In some embodiments the angiotensin converting enzyme inhibitor is captopril, enalapril, alacepril, delapril; ramipril, lisinopril, imidapril, benazepril, ceronapril, cilazapril, enalaprilat, fosinopril, moveltopril, perindopril, quinapril, spirapril, temocapril or trandolapril. In some embodiments the agent is an insulin secretion enhancer. In some embodiments the insulin secretion enhancer is tolbutamide; chlorpropamide; tolazamide; acetohexamide; glycopyramide; glibenclamide; gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolcyclamide, glimepiride, nateglinide, or mitiglinide. In some embodiments the agent is a thiazolidinedione. In some embodiments the thiazolidinedione is rosiglitazone or pioglitazone. In some embodiments the thiazolidinedione is rosiglitazone. In some embodiments the metabolic-related disorder is dyslipidemia, atherosclerosis, coronary heart disease, insulin resistance, obesity, impaired glucose tolerance, atheromatous disease, hypertension, stroke, Syndrome X, heart disease and type 2 diabetes. In some embodiments the metabolic-related disorder is dyslipidemia, atherosclerosis, coronary heart disease, insulin resistance and type 2 diabetes. In some embodiments the metabolic-related disorder is dyslipidemia. In some embodiments the metabolic-related disorder is atherosclerosis. In some embodiments the metabolic-related disorder is coronary heart disease. In some embodiments the metabolic-related disorder is insulin resistance. In some embodiments the metabolic-related disorder is type 2 diabetes.

One aspect of the present invention encompasses a method of producing a pharmaceutical composition comprising admixing at least one compound according to Formula (I), as described herein, and a pharmaceutically acceptable carrier or excipient.

Applicant reserves the right to exclude any one or more of the compounds from any of the embodiments of the invention. Applicant additionally reserves the right to exclude any metabolic-related disorder from any of the embodiments of the invention.

These and other aspects of the invention disclosed herein will be set forth in greater detail as the patent disclosure proceeds.

### BRIEF DESCRIPTION OF THE DRAWINGS

FIG. 1. FIG. 1 depicts a histogram representing relative expression levels of hRUP25 detected in different human tissues via DNA microarray. The horizontal axis displays the different tissues, identified in vertical text above the bar. The vertical axis indicates level of expression of hRUP25. In FIG. 1, note the high level of expression in primary adipocytes of hRUP25 (designated by the symbol “*”).

FIG. 2. FIG. 2 depicts melanophores transfected with DNA plasmids expressing hRUP25 without treatment These cells are pigment-aggregated because hRUP25 are Gi-coupled receptors having-a high basal level of activity, and therefore driving the aggregation to a measurable level in the absence of a ligand.

FIGS. 3A-B. FIGS. 3A and 3B illustrate the dose-dependant, nicotinic acid induced aggregation response of melanophores transfected with increasing amounts of plasmid DNA encoding hRUP25 (FIG. 3A). Cells transfected with 10 μg of plasmid DNA encoding hRUP25, respond to nicotinic acid with an EC₅₀ of about 54 nM.

As negative controls, FIG. 3B depicts melanophores transfected with either salmon sperm DNA (Mock) or plasmid DNA encoding the α_{2A}AR As is evident there is no aggregation response in these cells upon nicotinic acid treatment at doses up to 10 μM.

FIG. 4. FIG. 4 illustrates the nicotinic acid induced-inositol phosphates (IPs) accumulation in HEK293 cells co-expressing hRUP25 and the chimeric Gαq-subunit in which the last five amino acids have been replaced with the corresponding amino acids of Gαi (GqΔGi). This construct has been shown to convert the signaling of a Gi-coupled receptor to the Gq pathway (i.e. accumulation of inositol phosphates) in response to receptor activation. Cells transfected with GqΔGi plus either empty plasmid or the constitutively activated α_{2A}AR (α_{2A}K) served as controls for the IP assay which are non-responsive to nicotinic acid.

FIG. 5A. FIG. 5A is a set of immunofluorescent photomicrographs illustrating the expression of hemaglutinin (HA)-tagged hRUP25 in a stably transfected line of CHO cells (top; clone #46). No significant labeling is detected in mock stably-transfected CHO cells (Mock). The lower panels identify the nuclear (DAPI) staining of cells in the same field.

FIG. 5B. FIG. 5B illustrates nicotinic acid and nicotine induced-inhibition of forskolin stimulated cAMP accumulation in hRUP25-CHO cell stable line #46 (described in preceding paragraph). The EC₅₀ for nicotinic acid is 23.6 nM and that for nicotine is 9.8 μM.

FIG. 6. FIG. 6 indicates that, in response to nicotinic acid, both hRUP25 and the mouse ortholog mRUP25 can inhibit TSHR stimulated cAMP production (in the presence and absence of TSH).

FIG. 7. FIG. 7 shows the saturation binding curves of [³H]nicotinic acid ([³H]NA) to membranes prepared from HEK293 cells transiently expressing either hRUP25 or mRUP25. Note the significant binding of [³H]NA relative to either that found in membranes derived from mock transfected cells or in the presence of an excess of non-labeled nicotinic acid (200 μM).

FIG. 8. FIG. 8 is a table comparing the rank order of potency of various compounds on hRUP25 and the pharmacologically defined nicotinic acid receptor. The potencies at hRUP25 derived both by a functional analysis measuring the inhibition of forskolin induced cAMP production and competitive radioligand binding assays, closely match the order of potencies of the pharmacologically defined nicotinic acid receptor.

FIGS. 9A-B. FIG. 9A depicts nicotinic acid and related compounds inhibiting isoproterenol induced lipolysis in rat epidimal fat derived adipocytes at a concentration of 10 μM. P-3-T represents 3-tetrazole-5-pyridine. FIG. 9B illustrates a nicotinic acid dose-dependent inhibition of isoproterenol induced-lipolysis in rat epidimal fat derived adipocytes. Note the rightward shift in the dose-response curves with increasing concentrations of nicotinic acid.

FIG. 10. FIG. 10 illustrates the ability of both nicotinic acid and the related compound P-3-T (3-tetrazole-5-pyridine) to inhibit isoproterenol induced lipolysis in adipocyte primary cultures derived from human subcutaneous fat in a dose-dependant manner. The EC₅₀ value for nicotinic acid and P-3-T were 716 nM and 218 nM respectively.

### DETAILED DESCRIPTION OF THE INVENTION

### DEFINITIONS

The scientific literature that has evolved around receptors has adopted a number of terms to refer to ligands having various effects on receptors. For clarity and consistency, the following definitions will be used throughout this patent document. To the extent that these definitions conflict with other definitions for these terms, the following definitions shall control:

AFFINITY REAGENTS shall mean compounds that specifically and measurably bind to a target molecule. Preferably the target molecule is a GPCR of the invention. Preferably the AFFINITY REAGENTS are labeled to facilitate detection.

AGONISTS shall mean materials (e.g., ligands, candidate compounds) that activate an intracellular response when they bind to the receptor. In some embodiments, AGONISTS are those materials not previously known to activate the intracellular response when they bind to the receptor (e.g. to enhance GTPγS binding to membranes or to lower intracellular cAMP level). In some embodiments, AGONISTS are those materials not previously known to inhibit lipolysis when they bind to the receptor.

ALLOSTERIC MODULATORS shall mean materials (e.g., ligands, candidate compounds) that affect the functional activity of the receptor but which do not inhibit the endogenous ligand from binding to the receptor. Allosteric modulators include inverse agonists, partial agonists and agonists.

ANTAGONISTS shall mean materials (e.g., ligands, candidate compounds) that competitively bind to the receptor at the same site as the agonists but which do not activate an intracellular response, and can thereby inhibit the intracellular responses elicited by agonists. ANTAGONISTS do not diminish the baseline intracellular response in the absence of an agonist. In some embodiments, ANTAGONISTS are those materials not previously known to compete with an agonist to inhibit the cellular response when they bind to the receptor, e.g. wherein the cellular response is GTPγS binding to membranes or to the lowering of intracellular cAMP level.

ANTILIPOLYTIC GPCR shall mean a GPCR expressed by adipocytes and coupled to Gi or a Gi-coupled GPCR belonging to the nicotinic acid receptor sub-family of GPCRs. Activation of a Gi-coupled GPCR on adipocytes lowers intracellular cAMP levels, resulting in an inhibition of hormone sensitive lipase activity.

ATHEROSCLEROSIS is intended herein to encompass disorders of large and medium-sized arteries that result in the progressive accumulation within the intima of smooth muscle cells and lipids.

CHEMICAL GROUP, MOIETY or RESIDUE shall have the following meaing in the specification and Formulae described herein:

The term “C₁₋₅ alkylene” refers to a divalent branched or straight carbon group consisting of 1 to 5 carbon atoms, such as, —CH₂—, —CH₂CH₂—, —CH(CH₃)—, —CH₂CH₂CH₂—, —CH₂CH(CH₃)—, —CH₂CH₂CH₂CH₂—, and the like. In some embodiments the “C₁₋₅ alkylene” group contains one double bond, one triple bond or a carbonyl group whereby the one single bond of the “C₁₋₅ alkylene” group is replaced by a double bond or triple bond, such as, —CH═CH—, —C(CH₃)═CH—, —CH═C(CH₃)—, —CH₂CH═CH—, —CH₂CH₂CH═CH—, —CH═CHCH₂CH₃—, and the like, when a double bond is present it can be cis, trans or a mixture of both. In some embodiments, one carbon of the “C₁₋₅ alkylene” group is the carbon of a carbonyl group, such as, —C(O)—, —CH₂C(O)—, —C(O)CH₂—, —C(O)CH(CH₃)—, and the like.

The term “C₂₋₄ alkenyl” denotes a radical containing 2 to 4 carbons and at least one double bond. Some embodiments have 2 carbons. Examples of an alkenyl include vinyl, allyl, 2-butenyl, 3-butenyl and the like. Furthermore, the term “alkenyl” includes pure cis and trans isomers as well as mixtures thereof.

The term “C₁₋₄ alkoxy” as used herein denotes a radical alkyl, defined above, attached directly to an oxygen atom such as methoxy, ethoxy, n-propoxy, iso-propoxy, n-butoxy, t-butoxy, iso-butoxy and the like.

The term “C₁₋₈ alkyl” denotes a radical containing 1 to 8 carbons unless otherwise specified. Some embodiments are 1 to 6 carbons, some embodiments are 1 to 4 carbons, some embodiments are 1 to 3 carbons, some embodiments are 1 to 2 carbons, and some embodiments have 1 carbon. Examples of an alkyl include methyl, ethyl, n-propyl, iso-propyl, n-butyl, sec-butyl, t-butyl and the like.

The term “C₁₋₄ alkylamino” denotes an amino substituted with one group selected from alkyl containing 1 to 4 carbon atoms. Some examples include methylamino, ethylamino, and the like.

The term “di-C₁₋₄-alkylamino” denotes an amino substituted with two alkyl radicals that can be same or different wherein the alkyls group can contain 1 to 4 carbon atoms. Some examples include dimethylamino, methylethylamino, diethylamino and the like.

The term “C₂₋₄ alkynyl” denotes a radical containing 2 to 4 carbons and at least one triple bond. Some embodiments have 2 carbons. Examples of an alkynyl include ethynyl, 1-propynyl, 2-propynyl, 1-butynyl, 2-butynyl, 3-butynyl and the like. The term “C₁₋₄ alkylsulfinyl” denotes a sulfoxide, i.e., —S(O)—, radical containing 1 to 4 carbons, linear or branched. Examples include methylsulfonyl, ethylsulfinyl and the like.

The term “C₁₋₄ alkylsulfonyl” denotes a sulfone, i.e., —S(O)₂—, radical containing 1 to 4 carbons, linear or branched. Examples include methylsulfonyl, ethylsulfonyl and the like.

The term “C₁₋₄ alkylthio” denotes a sulfide, i.e., —S—, radical containing 1 to 4 carbons, linear or branched. Examples include methylsulfide, ethylsulfide, isopropylsulfide and the like.

The term “amino” denotes the group —NH₂.

The term “cyano” denotes the group —CN.

The term “halo” or “halogen” denotes to a fluoro, chloro, bromo or iodo group.

The term “C₁₋₄ haloalkoxy” denotes a haloalkyl, as defined above, that is directly attached to an oxygen to form a difluoromethoxy, trifluoromethoxy, 2,2,2-trifluoroethoxy, pentafluoroethoxy and the like.

The term “C₁₋₄ haloalkyl” denotes an alkyl group as defined above that is substituted with one or more halogens, preferably fluorine, such as a fluoromethyl, difluoromethyl, trifluoromethyl, 2,2,2-trifluoroethyl, pentafluoroethyl and the like.

The term “C₁₋₄ haloalkylsulfinyl” denotes a sulfoxide, i.e., —S(O)—, radical containing 1 to 4 carbons substituted with one or more halogens, linear or branched. Examples include trifluoromethylsulfinyl, 2,2,2-trifluoroethylsulfinyl, 2,2-difluoroethylsulfinyl and the like.

The term “C₁₋₄ haloalkylsulfonyl” denotes a sulfone, i.e., —S(O)₂—, radical containing 1 to 4 carbons, linear or branched substituted with one or more halogens. Examples include trifluoromethylsulfonyl, 2,2,2-trifluoroethylsulfonyl, 2,2-difluoroethylsulfonyl and the like.

The term “C₁₋₄ haloalkylthio” denotes an alkylthio radical substituted with one or more halogens. Examples include trifluoromethylthio, 1,1-difluoroethylthio, 2,2,2-trifluoroethylthio and the like.

The term “heteroaryl” denotes 5 or 6-membered aromatic rings having at least 1, 2, 3 or 4 heteroatoms in the ring, examples include, but are limited to, 1,3,4-oxadiazole, 1,2,4-oxadiazole, triazole, pyrazole, pyrole, isoxazole, furane, thiophene, thiazole, oxazole, pyridyl, pyrazinyl, pyridazinyl, pyrimidinyl, triazinyl, and the like; in some embodiments the “heteroaryl” is further substituted with substituents as described herein.

The term “hydroxyl” denotes the group —OH.

The term “nitro” denotes the group —NO₂.

The term “phenyl” denotes the C₆H₅-group, in some embodiments the “phenyl” is further substituted with substituents as described herein.

The term “thiol” denotes the group —SH.

The variable “X” in the Formulae found in this disclosure is selected from the group consisting of —NR₃C(O)—, —C(O)NR₃, —NR₃S(O)₂—, —S(O)₂NR₃—, —NR₃C(O)NR₄—, —NR₃C(O)O—, —OC(O)NR₃—, —NR₃—, —C(O)—, —CH(OH)—, —C(NH)—, —O—, —S—, —S(O)— and —S(O)₂— and are represented respectively by the following:

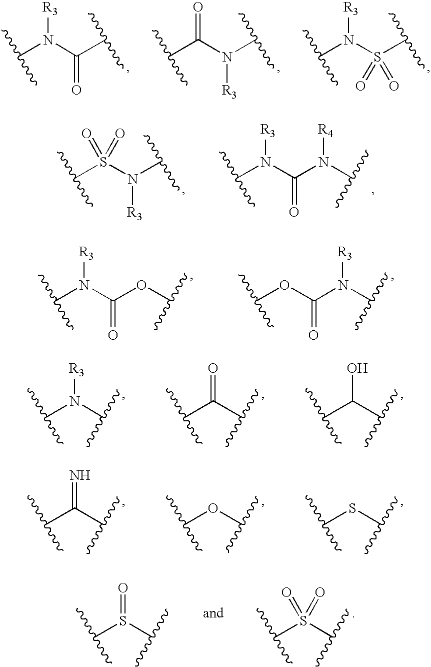

COMPOSITION means a material comprising at least one component; a “pharmaceutical composition” is an example of a composition.

COMPOUND EFFICACY shall mean a measurement of the ability of a compound to inhibit or stimulate receptor functionality; i.e. the ability to activate/inhibit a signal transduction pathway, in contrast to receptor binding affinity. Exemplary means of detecting compound efficacy are disclosed in the Example section of this patent document.

COMPRISING, CONSISTING ESSENTIALLY OF, and CONSISTING OF are defined herein according to their standard meaning. A defined meaning set forth in the M.P.E.P. controls over a defined meaning in the art and a defined meaning set forth in controlling Federal Circuit case law controls over a meaning set forth in the M.P.E.P.

CONTACT or CONTACTING shall mean bringing at least two moieties together, whether in an in vitro system or an in vivo system. Thus, “contacting” a RUP25 receptor with a compound of the invention includes the administration of a compound of the present invention to an individual, preferably a human, having a RUP25 receptor, as well as, for example, introducing a compound of the invention into a sample containing a cellular or more purified preparation containing a RUP25 receptor.

CORONARY HEART DISEASE is intended herein to encompass disorders comprising a narrowing of the small blood vessels that supply blood and oxygen to the heart. CORONARY HEART DISEASE usually results from the build up of fatty material and plaque. As the coronary arteries narrow, the flow of blood to the heart can slow or stop. CORONARY HEART DISEASE can cause chest pain (stable angina), shortness of breath, heart attack, or other symptoms.

DECREASE is used to refer to a reduction in a measurable quantity and is used synonymously with the terms “reduce”, “diminish”, “lower”, and “lessen”.

DIABETES as used herein is intended to encompass the usual diagnosis of DIABETES made from any of the methods including, but not limited to, the following list: symptoms of diabetes (e.g., polyuria, polydipsia, polyphagia) plus casual plasma glucose levels of greater than or equal to 200 mg/dl, wherein casual plasma glucose is defined any time of the day regardless of the timing of meal or drink consumption; 8 hour fasting plasma glucose levels of less than or equal to 126 mg/dl; and plasma glucose levels of greater than or equal to 200 mg/dl 2 hours following oral administration of 75 g anhydrous glucose dissolved in water.

DISORDERS OF LIPID METABOLISM are intended herein to include, but not be limited to, dyslipidemia.

DYSLIPIDEMIA is intended herein to encompass disorders comprising any one of elevated level of plasma free fatty acids, elevated level of plasma cholesterol, elevated level of LDL-cholesterol, reduced level of HDL-cholesterol, and elevated level of plasma triglycerides.

IN NEED OF PROPHYLAXIS OR TREATMENT as used herein refers to a judgment made by a caregiver (e.g. physician, nurse, nurse practitioner, etc. in the case of humans; veterinarian in the case of animals, including non-human mammals) that an individual or animal requires or will benefit from prophylaxis or treatment. This judgment is made based on a variety of factors that are in the realm of a caregiver's expertise, but that includes the knowledge that the individual or animal is ill, or will be ill, as the result of a disease, condition or disorder that is treatable by the compounds of the invention. In general, “in need of prophylaxis” refers to the judgment made by the caregiver that the individual will become ill. In this context, the compounds of the invention are used in a protective or preventive manner. However, “in need of treatment” refers to the judgment of the caregiver that the individual is already ill, therefore, the compounds of the present invention are used to alleviate, inhibit or ameliorate the disease, condition or disorder.

INDIVIDUAL as used herein refers to any animal, including mammals, preferably mice, rats, other rodents, rabbits, dogs, cats, swine, cattle, sheep, horses, or primates, and most preferably humans.

INHIBIT or INHIBITING, in relationship to the term “response” shall mean that a response is decreased or prevented in the presence of a compound as opposed to in the absence of the compound.

INSULIN RESISTANCE as used herein is intended to encompass the usual diagnosis of insulin resistance made by any of a number of methods, including but not restricted to: the intravenous glucose tolerance test or measurement of the fasting insulin level. It is well known that there is an excellent correlation between the height of the fasting insulin level and the degree of insulin resistance. Therefore, one could use elevated fasting insulin levels as a surrogate marker for insulin resistance for the purpose of identifying which normal glucose tolerance (NGT) individuals have insulin resistance. A diagnosis of insulin resistance can also be made using the euglycemic glucose clamp test.

INVERSE AGONISTS shall mean materials (e.g., ligand, candidate compound) that bind either to the endogenous form or to the constitutively activated form of the receptor so as to reduce the baseline intracellular response of the receptor observed in the absence of agonists.

KNOWN RECEPTOR shall mean an endogenous receptor for which the endogenous ligand specific for that receptor has been identified.

LIGAND shall mean a molecule specific for a naturally occurring receptor.

METABOLIC-RELATED DISORDERS are intended herein to include, but not be limited to, dyslipidemia, atherosclerosis, coronary heart disease, insulin resistance, obesity, impaired glucose tolerance, atheromatous disease, hypertension, stroke, Syndrome X, heart disease and type 2 diabetes.

As used herein, the terms MODULATE or MODIFY are meant to refer to an increase or decrease in the amount, quality, or effect of a particular activity, function or molecule.

As used herein, the term NICOTINIC ACID ANALOG OR DERIVATIVE is meant to molecules which bind to nicotinic acid receptors and have substantially similar effects on the receptor. Such analogs and derivatives are well-known to those skilled in the art and include, but are not limited to, acipimox and niacinamide.

PARTIAL AGONISTS shall mean materials (e.g., ligands, candidate compounds) that activate the intracellular response when they bind to the receptor to a lesser degree/extent than do full agonists.

PHARMACEUTICAL COMPOSITION shall mean a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

STIMULATE or STIMULATING, in relationship to the term “response” shall mean that a response is increased in the presence of a compound as opposed to in the absence of the compound.

SUBJECT shall mean primates, including but not limited to humans and baboons, as well as pet animals such as dogs and cats, laboratory animals such as rats and mice, and farm animals such as horses, sheep, and cows.

SUBSTANTIALLY shall refer to a result which is within 40% of a control result, preferably within 35%, more preferably within 30%, more preferably within 25%, more preferably within 20%, more preferably within 15%, more preferably within 10%, more preferably within 5%, more preferably within 2%, and most preferably within 1% of a control result For example, in the context of receptor functionality, a test receptor may exhibit substantially similar results to a control receptor if the transduced signal, measured using a method taught herein or similar method known to the art-skilled, is within 40% of the signal produced by a control signal.

The order of the following sections is set forth for presentational efficiency and is not intended, nor should be construed, as a limitation on the disclosure or the claims to follow.

THERAPEUTICALLY EFFECTIVE AMOUNT as used herein refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) Preventing the disease; for example, preventing a disease, condition or disorder in an individual that can be predisposed to the disease, condition or disorder but does not yet experience or display the pathology or symptomatology of the disease,

(2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and

(3) Ameliorating the disease; for example, ameliorating a disease, condition or disorder in an individual that is experiencing or displaying the pathology or symptomatology of the disease, condition or disorder (i.e., reversing the pathology and/or symptomatology).

### COMPOUNDS OF THE INVENTION

One aspect of the present invention encompasses pyrazole carboxylic acid and ester derivatives as shown in Formula (I): 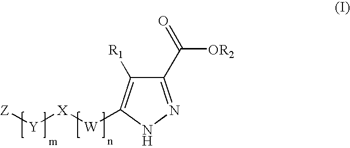
or a pharmaceutically acceptable salt, solvate or hydrate thereof; wherein Z, Y, X, W, m, n, R₁, and R₂ have the same definitions as described herein, supra and infra.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

It is understood and appreciated that compounds of the invention may have one or more chiral centers, and therefore can exist as enantiomers and/or diastereomers. The invention is understood to extend to and embrace all such enantiomers, diastereomers and mixtures thereof, including, but not limited to, racemates. Accordingly, some embodiments of the present invention pertain to compounds of Formula (I) and formulae used throughout this disclosure that are R enantiomers. Further, some embodiments of the present invention pertain to compounds of Formula (I) and formulae used throughout this disclosure that are S enantiomers. When more than one chiral center is present, for example two chiral centers then, some embodiments of the present invention include compounds that are RS or SR enantiomers. In further embodiments, compounds of the present invention are RR or SS enantiomers. It is understood that compounds of Formula (I) and formulae used throughout this disclosure are intended to represent all individual enantiomers and mixtures thereof, unless stated or shown otherwise.

Compounds of the invention can also include tautomeric forms, such as keto-enol tautomers, and the like. Tautomeric forms can be in equilibrium or sterically locked into one form by appropriate substitution. It is understood that the various tautomeric forms are within the scope of the compounds of the present invention.

Compounds of the invention can also include all isotopes of atoms occurring in the intermediates and/or final compounds. Isotopes include those atoms having the same atomic number but different mass numbers. For example, isotopes of hydrogen include deuterium and tritium.

In some embodiments, when both R₁ and R₂ are H then —[W]ₙ—X—[y]ₘ-Z together is not CO₂H, C(O)—C₆H₄-p-O—C₈H₁₇, OCH₂CH₃, OH, CH₂CH₂CH₂CH₂CO₂H, CH₂CH₂CH₂CO₂H, CH₂CO₂H and CH₂CH₂CO₂H.

In some embodiments, when R₁ is CH₃ and R₂ is H then —[W]ₙ—X—[Y]ₘ-Z together is not CH₂CO₂H, C(O)CH═CH C₆H₅, C(O)C₆H₄-p-OCH₃, CO₂H, C(O)CH₃, C(O)C₆H₄-o-CH₃, C(O)C₆H₄-o-Br, C(O)C₆H₄-o-Cl, and C(O)C₆H₅.

In some embodiments, when R₁ is Br and R₂ is H then —[W]ₙ—X—[Y]ₘ-Z together is not CO₂H.

In some embodiments, when R₁ is OH and R₂ is H then —[W]ₙ—X—[Y]ₘ-Z together is not CO₂H.

In some embodiments, when R₁ is H and R₂ is CH₃ then —[W]ₙ—X—[Y]ₘ-Z together is not 2,6-dichloro-4-trifluoromethylphenoxy, C(O)NH—C₆H₄-p-OCH₂CH₃, NHC(O)CH(CH₃)₂, SCH₃, C(O)—C₆H-p-O—C₈H₁₇, SCH₂CH₃, C(O)NH₆H₅, CH(OCH₃)₂, CH₂OC(O)CH₃, CO₂H, CO₂CH₃, C(O)C₆H₄-p-NO₂, C(O)C₆H₅, CH₂CH₂CO₂CH₃, CH₂CH₂CH₂CH₂CO₂CH₃, CH₂CH₂CH₂CO₂CH₃ and CH₂CO₂CH₃.

In some embodiments, when R₁ is OH and R₂ is CH₃ then —[W]ₙ—X—[Y]ₘ-Z together is not CH₂OCH₂C₆H₅, CH₂OCH(CH₃)₂ and CH₂OH.

In some embodiments, when R₂ is CH₃ then:
R₁ is not CH₃ and —[W]ₙ—X—[Y]ₘ-Z together is not 2,6-dichloro-4-trifluoromethylphenoxy;
R₁ is not I and —[W]ₙ—X—[Y]ₘ-Z together is not CO₂C(CH₃)₃;
R₁ is not C(CH₃)₃ and —[W]ₙ—X—[Y]ₘ-Z together is not formyl;
R₁ is not Br and —[W]ₙ—X—[Y]ₘ-Z together is not CO₂CH₃; and
R₁ is not CH₂CH₂CH₂CH₃ and —[W]ₙ—X—[Y]ₘ-Z together is not formyl.

In some embodiments, when R₁ is H and R₂ is CH₂CH₃ then —[W]ₙ—X—[Y]ₘ-Z together is not CH₂SCH₂CH₃, OCH₂CH₂CH═CH₂, CH₂CH₂CH₂OH, CH₂CH₂CHO, CO₂CH₂CH₃, OCH₃, C(O)CH₂Br, CO₂C₈H₁₇, formyl, OH, CH₂N(CH₂CH₂Cl)₂, CH(CH₃)OC(O)CH₃, CH₂OH, CH₂OC(O)CH₃, C(O)CH₃, C(O)C6H₅ and C(O)NHCH₂CO₂CH₂CH₃.

In some embodiments, when R₁ is CH₃ and R₂ is CH₂CH₃ then —[W]ₙ—X—[Y]ₘ-Z together is not CH(OH)C₆H₄-p-N(CH₃)₂, C(O)CH₂C(O)CH₃, CO₂CH₂C₆H₅, CO₂CH₃, C(O)CH₂CH₂CH₃, C(O)CH₃, C(O)C₆H₄-p-OCH₃, C(O)C₆H₄-o-Br, C(O)C₆H₄-p-Cl, C(O)C₆H₄-o-Cl, C(O)CH₂C₆H₅ and C(O)C₆H₅.

In some embodiments, when R₂ is CH₂CH₃ then:
R₁ is not I and —[W]ₙ—X—[Y]ₘ-Z together is not CO₂CH₂CH₃;
R₁ is not CF₃ and —[W]ₙ—X—[Y]ₘ-Z together is not CO₂CH₂CH₃; and
R₁ is not Br and —[W]ₙ—X—[Y]ₘ-Z together is not CO₂CH₂CH₃.

In some embodiments, when R₁ is OH and R₂ is CH₂CH₃ then —[W]ₙ—X—[Y]ₘ-Z together is not C(O)C₆H₅, C(O)NH₂ and CO₂CH₂CH₃.

In some embodiments, when R₁ is H and R₂ is C(CH₃)₃ then —[W]ₙ—X—[Y]ₘ-Z together is not CO₂C(CH₃)₃, C(O)NHC(O)CH₃ and C(O)NH₂.

In some embodiments, when R₁ is OH and R₂ is CH₂CH₂CH₂CH₃ then —[W]ₙ—X—[Y]ₘ-Z together is not C(O)C₆H₅.

Some embodiments of the present invention pertain to compounds of Formula (I) wherein “n” is 0. In some embodiments, compounds of the invention can be represented by Formula (Ia) as illustrated below: 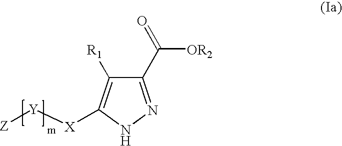
wherein each variable in Formula (Ia) has the same meaning as described herein, supra and infra.

Some embodiments of the present invention pertain to compounds of Formula (I) wherein “n” is 1. In some embodiments, compounds of the invention can be represented by Formula (Ib) as illustrated below: 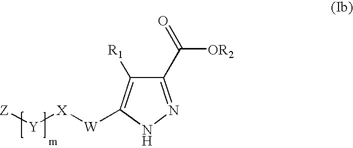
wherein each variable in Formula (Ib) has the same meaning as described herein, supra and infra.

Some embodiments of the present invention pertain to compounds of Formula (I) wherein “m” is 0. In some embodiments, compounds of the invention can be represented by Formula (Ic) as illustrated below: 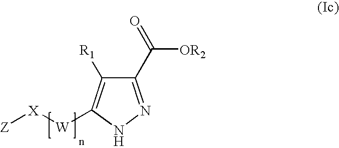
wherein each variable in Formula (Ic) has the same meaning as described herein, supra and infra.

Some embodiments of the present invention pertain to compounds of Formula (I) wherein “m” is 1. In some embodiments, compounds of the invention can be represented by Formula (Id) as illustrated below: 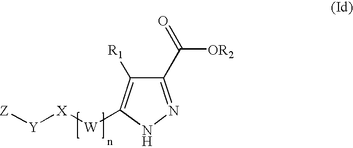
wherein each variable in Formula (Id) has the same meaning as described herein, supra and infra.

Some embodiments of the present invention pertain to compounds of Formula (I), more specifically Formulae (Ib), (Ic) or (Id), wherein W is the straight or branched chain C₁₋₅ alkylene group optionally containing one double bond, one triple bond or carbonyl, wherein said C₁₋₅ alkylene group is optionally substituted with halogen, hydroxyl, C₁₋₄ alkyl or C₁₋₄ alkoxy. The C₁₋₅ alkylene group is a straight chain group of 1 to 5 carbons. In some embodiments the alkylene chain consists of single bonds. In some embodiments two adjacent carbons in this chain can be bonded together by a double bond or a triple bond, in still other embodiments, a single carbon can be bonded to an oxygen by a double bond thus forming a carbonyl group as represented in some embodiments disclosed herein [i.e., —C(═O)—].

The C₁₋₅ alkylene group, when present (i.e., n=1), can be optionally substituted with halogen, hydroxyl, C₁₋₄ alkyl or C₁₋₄ alkoxy. The number of substituents on the C₁₋₅ alkylene group depends on the specific group present, for example, when the C₁₋₅ alkylene group is —CH₂— the number of substituents can be 1 or 2. Whereas, when the C₁₋₅ alkylene group is —(CH₂)₄— then the number of substituents can range from 1 to 8.

Some embodiments of the present invention pertain to compounds of Formula (I) wherein W is —CH₂— optionally substituted with halogen, hydroxyl, C₁₋₄ alkyl or C₁₋₄ alkoxy. In one embodiment, W is —CH₂—. In some embodiments, compounds of the present invention can be represented by Formula (Ie) as illustrated below: 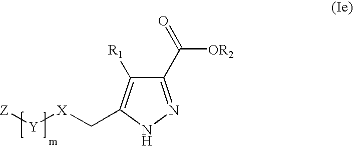
wherein each variable in Formula (Ie) has the same meaning as described herein, supra and infra. In some embodiments W is —CH(CH₃)— optionally substituted with halogen, hydroxyl or C₁₋₄ alkoxy. In one embodiment, W is —CH(CH₃)—. In some embodiments, compounds of the present invention can be represented by Formula (If) as illustrated below: 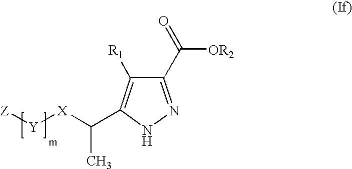
wherein each variable in Formula (If) has the same meaning as described herein, supra and infra. In some embodiments W is —C(CH₃)₂—. In some embodiments, compounds of the present invention can be represented by Formula (Ig) as illustrated below: 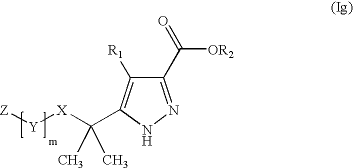
wherein each variable in Formula (Ig) has the same meaning as described herein, supra and infra.

Some embodiments of the present invention pertain to compounds of Formula (I) wherein W is —CH₂CH₂— optionally substituted with halogen, hydroxyl, C₁₋₄ alkyl or C₁₋₄ alkoxy. In one embodiment, W is —CH₂CH₂—. In some embodiments, compounds of the present invention can be represented by Formula (Ih) as illustrated below: 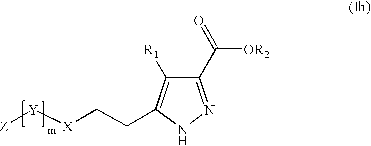
wherein each variable in Formula (Ih) has the same meaning as described herein, supra and infra. In some embodiments W is —CH(CH₃)CH₂— or —CH₂CH(CH₃)— optionally substituted with halogen, hydroxyl, C₁₋₄ alkyl or C₁₋₄ alkoxy. In some embodiments W is —CH(CH₃)CH₂— or —CH₂CH(CH₃)—. In some embodiments, compounds of the present invention can be represented by Formulae (Ii) and (Ij) respectively as illustrated below: 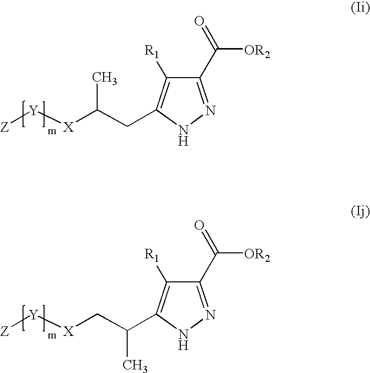
wherein each variable in Formulae (Ii) and (Ij) has the same meaning as described herein, supra and infra.

In some embodiments W is —C(CH₃)₂CH₂— or —CH₂C(CH₃)₂— optionally substituted with halogen, hydroxyl, C₁₋₄ alkyl or C₁₋₄ alkoxy. In some embodiments W is —C(CH₃)₂CH₂— or —CH₂C(CH₃)₂—. In some embodiments, compounds of the present invention can be represented by Formulae (Ik) and (Im) respectively as illustrated below: 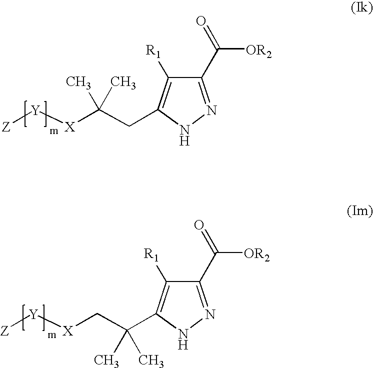
wherein each variable in Formulae (Ik) and (Im) has the same meaning as described herein, supra and infra.

In some embodiments W is —CH(OCH₃)CH₂— or —CH₂CH(OCH₃)— optionally substituted with halogen, hydroxyl or C₁₋₄ alkyl. In some embodiments W is —CH(OCH₃)CH₂— or —CH₂CH(OCH₃)—. In some embodiments, compounds of the present invention can be represented by Formulae (In) and (Io) respectively as illustrated below: 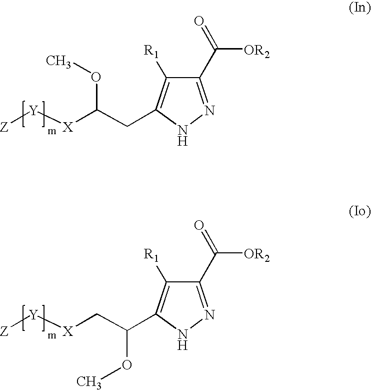
wherein each variable in Formulae (In) and (Io) has the same meaning as described herein, supra and infra.

Some embodiments of the present invention pertain to compounds of Formula (I) wherein W is —CH₂CH₂CH₂— optionally substituted with halogen, hydroxyl, C₁₋₄ alkyl or C₁₋₄ alkoxy. In some embodiments W is —CHClCH₂CH₂— or —CH₂CH₂CHCl—. In some embodiments W is —CH₂CH₂CH₂—. In some embodiments, compounds of the present invention can be represented by Formula (Ip) as illustrated below: 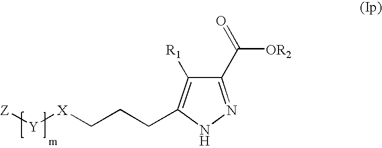
wherein each variable in Formula (Ip) has the same meaning as described herein, supra and infra.

Some embodiments of the present invention pertain to compounds of Formula (I) wherein W is —CH₂CH₂CH₂CH₂— optionally substituted with halogen, hydroxyl, C₁₋₄ alkyl or C₁₋₄ alkoxy. In some embodiments W is —CH₂CH₂CH₂CH₂—. In some embodiments, compounds of the present invention can be represented by Formula (Iq) as illustrated below: 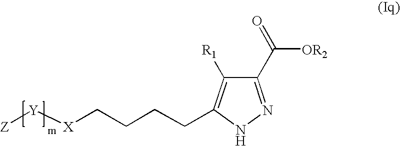
wherein each variable in Formula (Iq) has the same meaning as described herein, supra and infra.

Some embodiments of the present invention pertain to compounds of Formula (I) wherein W is —CH═CH— optionally substituted with C₁₋₄ alkyl or C₁₋₄ alkoxy. In some embodiments W is —CH═CH—. In some embodiments, compounds of the present invention can be represented by Formula (Ir) as illustrated below: 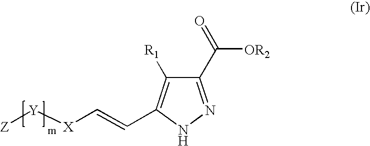
wherein each variable in Formula (Ir) has the same meaning as described herein, supra and infra. It is understood that when a double bond is present it can be either cis or trans; or a mixture of both cis and trans.

Some embodiments of the present invention pertain to compounds of Formula (I) wherein W is —C≡C—. In some embodiments, compounds of the present invention can be represented by Formula (Is) as illustrated below: 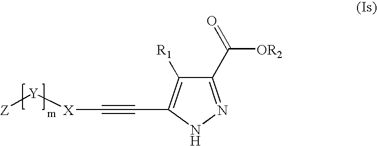
wherein each variable in Formula (Is) has the same meaning as described herein, supra and infra.

Some embodiments of the present invention pertain to compounds of Formula (I) wherein W is —C(O)—. In some embodiments, W is —C(O)— and X is —O—. In some embodiments, “m” is 1 thus forming an ester group. In some embodiments, “m” is 0 and Z is H thus forming a carboxylic acid group.

Some embodiments of the present invention pertain to compounds of Formula (I) wherein W is —CH₂C(O)— or —C(O)CH₂— optionally substituted with halogen, hydroxyl, C₁₋₄ alkyl or C₁₋₄ alkoxy. In some embodiments W is —CHFC(O)— or —C(O)CHF—. In some embodiments W is —CH(CH₃)C(O)— or —C(O)CH(CH₃)— optionally substituted with halogen, hydroxyl, C₁₋₄ alkyl or C₁₋₄ alkoxy. In some embodiments W is —C(OH)(CH₃)C(O)— or —C(O)C(OH)(CH₃). In some embodiments W is —C(CH₃)₂C(O)— or —C(O)C(CH₃)₂—. In some embodiments, compounds of the present invention can be represented by Formulae (It) and (Iu) respectively as illustrated below: 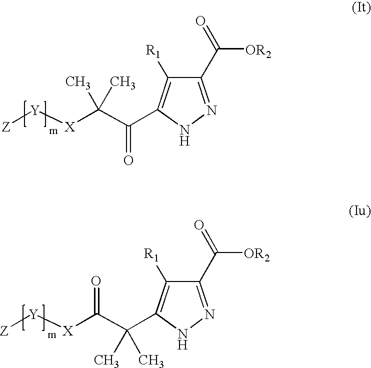
wherein each variable in Formulae (It) and (Iu) has the same meaning as described herein, supra and infra. Some embodiments of the present invention pertain to compounds of Formula (It) when X is —O— or —NR₃—. Some embodiments of the present invention include compounds of Formula (Iu) when X is —O—. In some embodiments W is —CH₂C(O)— or —C(O)CH₂—. In some embodiments, compounds of the present invention can be represented by Formulae (Iv) and (Iw) respectively as illustrated below: 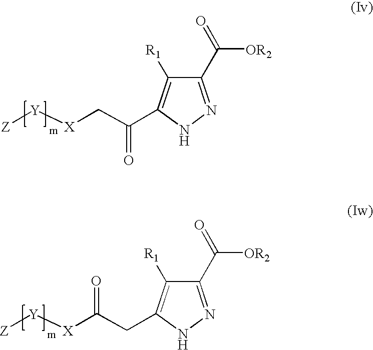
wherein each variable in Formulae (Iv) and (Iw) has the same meaning as described herein, supra and infra. Some embodiments of the present invention pertain to compounds of Formula (Iv) wherein X is —O— or —NR₃—. Some embodiments of the present invention pertain to compounds of Formula (Iw) when X is —O—.

Some embodiments of the present invention pertain to compounds of Formula (I) wherein W is —CH₂CH₂C(O)— or —C(O)CH₂CH₂— optionally substituted with halogen, hydroxyl, C₁₋₄ alkyl or C₁₋₄ alkoxy. In some embodiments W is —C(CH₃)₂CH₂C(O)— or —C(O)CH₂C(CH₃)₂— optionally substituted with halogen, hydroxyl, C₁₋₄ alkyl or C₁₋₄ alkoxy. In some embodiments W is —CH₂CH₂C(O)— or —C(O)CH₂CH₂—. In some embodiments, compounds of the present invention can be represented by Formulae (Ix) and (Iy) respectively as illustrated below: 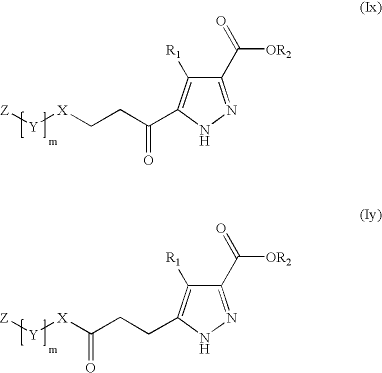
wherein each variable in Formulae (Ix) and (Iy) has the same meaning as described herein, supra and infra. Some embodiments of the present invention pertain to compounds of Formula (Ix) when X is —O— or —NR₃— and in still other embodiments R₃ is H or CH₃. Some embodiments of the present invention pertain to compounds of Formula (Iy) when X is —O—.

Some embodiments of the present invention pertain to compounds of Formula (I) wherein W is —CH₂C(O)CH₂— optionally substituted with halogen, hydroxyl, C₁₋₄ alkyl or C₁₋₄ alkoxy. In some embodiments W is —CH₂C(O)CH₂—. In some embodiments, compounds of the present invention can be represented by Formula (Iz) as illustrated below: 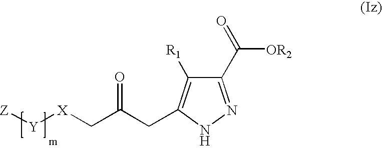
wherein each variable in Formulae (Iz) has the same meaning as described herein, supra and infra.

Some embodiments of the present invention pertain to compounds of Formula (I) wherein W is —CH₂CH₂CH₂C(O)— or —C(O)CH₂CH₂CH₂— optionally substituted with halogen, hydroxyl, C₁₋₄ alkyl or C₁₋₄ alkoxy. In some embodiments W is —CH(CH₃)CH₂CH₂C(O)— or —C(O)CH₂CH₂CH(CH₃)— optionally substituted with halogen, hydroxyl, C₁₋₄ alkyl or C₁₋₄ alkoxy. In some embodiments W is —CH₂CH₂CH₂C(O)— or —C(O)CH₂CH₂CH₂—. In some embodiments, compounds of the present invention can be represented by Formulae (IIa) and (IIb) respectively as illustrated below: 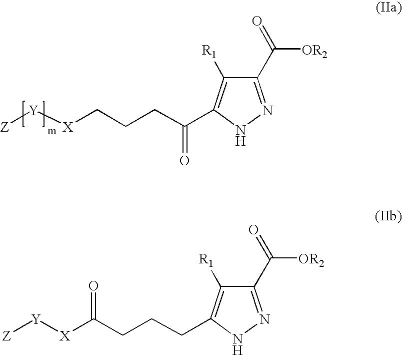
wherein each variable in Formulae (IIa) and (IIb) has the same meaning as described herein, supra and infra.

Some embodiments of the present invention pertain to compounds of Formula (I) wherein W is —CH₂CH₂C(O)CH₂— or —CH₂C(O)CH₂CH₂— optionally substituted with halogen, hydroxyl, C₁₋₄ alkyl or C₁₋₄ alkoxy. In some embodiments W is —CH₂CH₂C(O)CH₂— or —CH₂C(O)CH₂CH₂—. In some embodiments, compounds of the present invention can be represented by Formulae (IIc) and (IId) respectively as illustrated below: 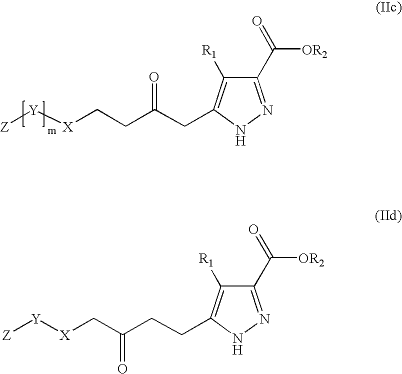
wherein each variable in Formulae (IIc) and (IId) has the same meaning as described herein, supra and infra.

Some embodiments of the present invention pertain to compounds of Formula (I) wherein W is —CH═CHC(O)— or —C(O)CH═CH— optionally substituted with halogen, hydroxyl, C₁₋₄ alkyl or C₁₋₄ alkoxy. In some embodiments W is —CH=CHC(O)— or —C(O)CH═CH—. In some embodiments, compounds of the present invention can be represented by Formulae (IIe) or (IIf) respectively as illustrated below: 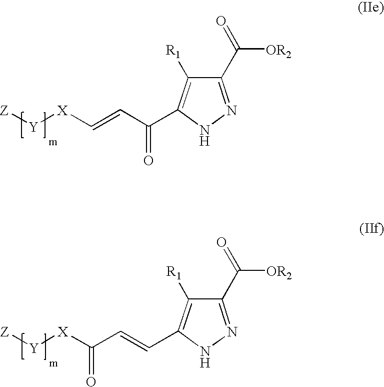
wherein each variable in Formulae (IIe) and (IIf) has the same meaning as described herein, supra and infra.

Some embodiments of the present invention pertain to compounds of Formula (I) wherein W is —C(CH₃)═CHC(O)— or —C(O)CH═C(CH₃)— optionally substituted with halogen, hydroxyl, C₁₋₄ alkyl or C₁₋₄ alkoxy. In one embodiment, W is —C(CH₃)═CHC(O)— or —C(O)CH═C(CH₃)—. In some embodiments, compounds of the present invention can be represented by either Formulae (IIg) or (IIh) respectively as illustrated below: 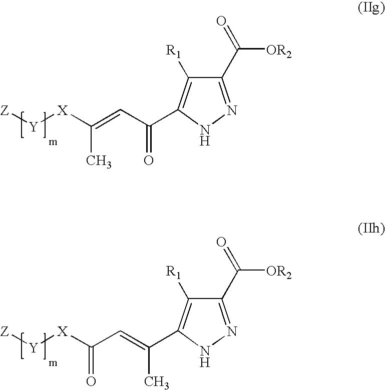
wherein each variable in Formulae (IIg) and (IIh) has the same meaning as described herein, supra and infra.

Some embodiments of the present invention pertain to compounds of Formula (I) wherein Y is the C₁₋₅ alkylene group optionally containing one double bond, one triple bond or carbonyl, wherein said C₁₋₅ alkylene group is optionally substituted with halogen, hydroxyl, C₁₋₄ alkyl or C₁₋₄ alkoxy. The C₁₋₅ alkylene group is a straight chain group of 1 to 5 carbons. In some embodiments the alkylene chain consists of single bonds. In some embodiments two adjacent carbons in this chain can be bonded together by a double bond or a triple bond, in still other embodiments, a single carbon can be bonded to an oxygen by a double bond thus forming a carbonyl group as represented in some embodiments disclosed herein, can be depicted as —C(═O)—.

Some embodiments of the present invention pertain to compounds of Formula (I) wherein Y is —CH₂— optionally substituted with halogen, hydroxyl, C₁₋₄ alkyl or C₁₋₄ alkoxy. In some embodiments Y is —CH₂—. In some embodiments, compounds of the present invention can be represented by Formula (IIi) as illustrated below: 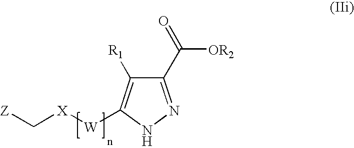
wherein each variable in Formula (IIi) has the same meaning as described herein, supra and infra. In some embodiments Y is —CH(CH₃)— optionally substituted with halogen, hydroxyl or C₁₋₄ alkoxy. In some embodiments Y is —C(CH₃)₂—. In some embodiments, compounds of the present invention can be represented by Formula (IIj) as illustrated below: 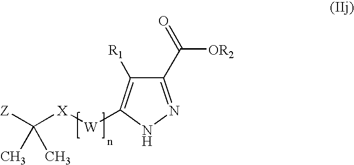
wherein each variable in Formula (IIj) has the same meaning as described herein, supra and infra.

Some embodiments of the present invention pertain to compounds of Formula (I) wherein Y is —CH₂CH₂— optionally substituted with halogen, hydroxyl, C₁₋₄ alkyl or C₁₋₄ alkoxy. In some embodiments Y is —CH₂CH₂—. In some embodiments, compounds of the present invention can be represented by Formula (IIk) as illustrated below: 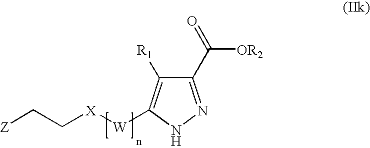
wherein each variable in Formula (IIk) has the same meaning as described herein, supra and infra. In some embodiments Y is —CH(CH₃)CH₂— or —CH₂CH(CH₃)— optionally substituted with halogen, hydroxyl, C₁₋₄ alkyl or C₁₋₄ alkoxy. In some embodiments Y is —CH(CH₃)CH₂— or —CH₂CH(CH₃)—. In some embodiments, compounds of the present invention can be represented by Formulae (IIm) and (IIn) respectively as illustrated below: 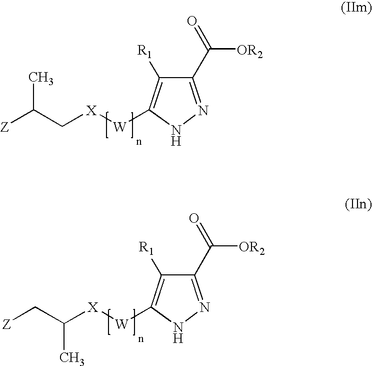
wherein each variable in Formulae (IIm) and (IIn) has the same meaning as described herein, supra and infra. In some embodiments Y is —C(CH₃)₂CH₂— or —CH₂C(CH₃)₂— optionally substituted with halogen, hydroxyl, C₁₋₄ alkyl or C₁₋₄ alkoxy. In some embodiments Y is C(CH₃)₂CH₂— or —CH₂C(CH₃)₂—. In some embodiments, compounds of the present invention can be represented by Formulae (IIo) and (IIp) respectively as illustrated below: 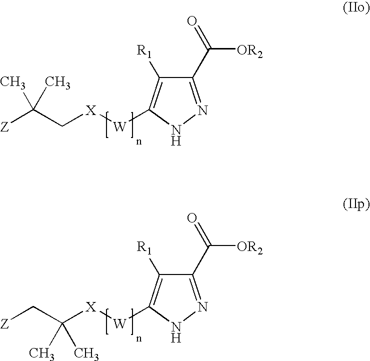
wherein each variable in Formulae (IIo) and (IIp) has the same meaning as described herein, supra and infra. In some embodiments Y is —CH(OCH₃)CH₂— or —CH₂CH(OCH₃)— optionally substituted with halogen, hydroxyl or C₁₋₄ alkyl. In some embodiments Y is —CH(OCH₃)CH₂— or —CH₂CH(OCH₃)—. In some embodiments, compounds of the present invention can be represented by Formulae (IIq) and (IIr) respectively as illustrated below: 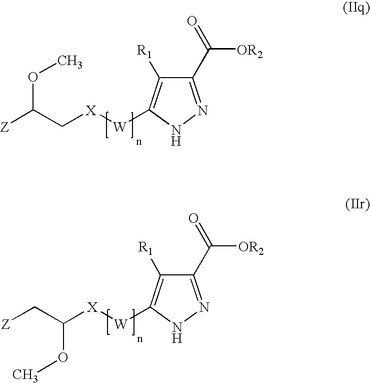
wherein each variable in Formulae (IIq) and (IIr) has the same meaning as described herein, supra and infra.

Some embodiments of the present invention pertain to compounds of Formula (I) wherein Y is —CH₂CH₂CH₂— optionally substituted with halogen, hydroxyl, C₁₋₄ alkyl or C₁₋₄ alkoxy. In some embodiments Y is —CH₂CH₂CH₂—. In some embodiments, compounds of the present invention can be represented by Formula (IIs) as illustrated below: 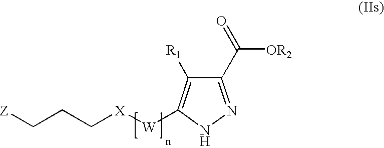
wherein each variable in Formula (IIs) has the same meaning as described herein, supra and infra.

Some embodiments of the present invention pertain to compounds of Formula (I) wherein Y is —CH₂CH₂CH₂CH₂— optionally substituted with halogen, hydroxyl, C₁₋₄ alkyl or C₁₋₄ alkoxy. In some embodiments Y is —CH₂CH₂CH₂CH₂—. In some embodiments, compounds of the present invention can be represented by Formula (IIt) as illustrated below: 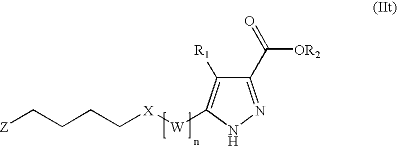
wherein each variable in Formula (IIt) has the same meaning as described herein, supra and infra.

Some embodiments of the present invention pertain to compounds of Formula (I) wherein Y is —CH═CH— optionally substituted with C₁₋₄ alkyl or C₁₋₄ alkoxy. In some embodiments Y is —CH═CH—. In some embodiments, compounds of the present invention can be represented by Formula (IIu) as illustrated below: 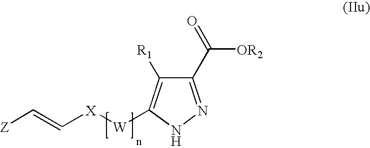
wherein each variable in Formula (IIu) has the same meaning as described herein, supra and infra.

Some embodiments of the present invention pertain to compounds of Formula (I) wherein Y is —C≡C—. In some embodiments, compounds of the present invention can be represented by Formula (IIv) as illustrated below: 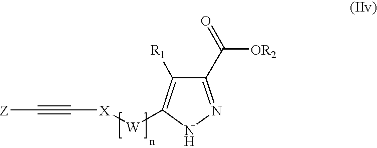
wherein each variable in Formula (IIv) has the same meaning as described herein, supra and infra.

Some embodiments of the present invention pertain to compounds of Formula (I) wherein Y is —C≡CCH₂— or —CH₂C≡C— optionally substituted with halogen, hydroxyl, C₁₋₄ alkyl or C₁₋₄ alkoxy. In some embodiments Y is —C≡CCH₂— or —CH₂C≡C—. In some embodiments, compounds of the present invention can be represented by Formulae (IIw) and (IIx) respectively as illustrated below: 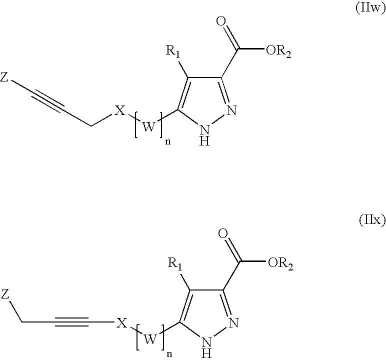
wherein each variable in Formulae (IIw) and (IIx) has the same meaning as described herein, supra and infra.

Some embodiments of the present invention pertain to compounds of Formula (I) wherein Y is —C(O)—. In some embodiments, compounds of the present invention can be represented by Formula (IIy) as illustrated below: 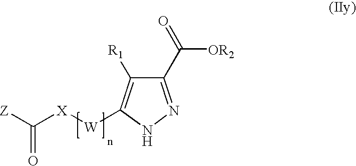
wherein each variable in Formula (IIy) has the same meaning as described herein, supra and infra.

Some embodiments of the present invention pertain to compounds of Formula (I) wherein Y is —CH₂C(O)— or —C(O)CH₂— optionally substituted with halogen, hydroxyl, C₁₋₄ allyl or C₁₋₄ alkoxy. In some embodiments Y is —CH(CH₃)C(O)— or —C(O)CH(CH₃)— optionally substituted with halogen, hydroxyl, C₁₋₄ alkyl or C₁₋₄ alkoxy. In some embodiments Y is —CH(CH₃)C(O)— or —C(O)CH(CH₃)—. In some embodiments, compounds of the present invention can be represented by Formulae (IIIa) and (IIIb) respectively as illustrated below: 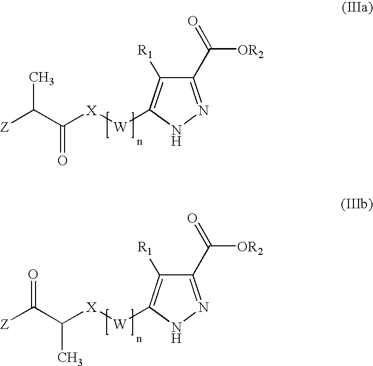
wherein each variable in Formulae (IIIa) and (IIIb) has the same meaning as described herein, supra and infra. In some embodiments Y is —C(CH₃)₂C(O)— or —C(O)C(CH₃)₂—. In some embodiments, compounds of the present invention can be represented by Formulae (IIIc) and (IIId) respectively as illustrated below: 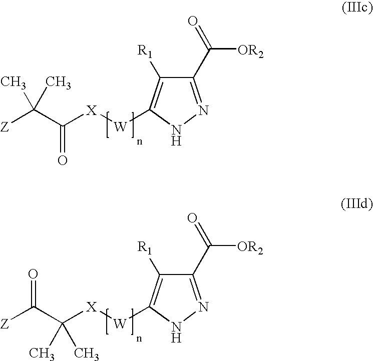
wherein each variable in Formulae (IIIc) and (IIId) has the same meaning as described herein, supra and infra. In some embodiments Y is —CH₂C(O)— or —C(O)CH₂—. In some embodiments, compounds of the present invention can be represented by Formulae (IIIe) and (IIIf) respectively as illustrated below: 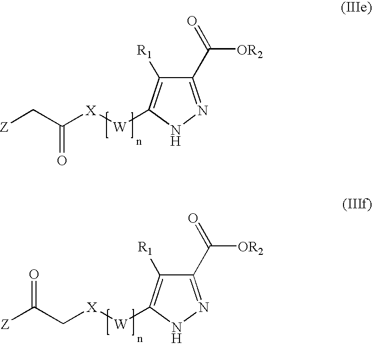
wherein each variable in Formulae (IIIe) and (IIIf) has the same meaning as described herein, supra and infra.

Some embodiments of the present invention pertain to compounds of Formula (I) wherein Y is —CH₂CH₂C(O)— or —C(O)CH₂CH₂— optionally substituted with halogen, hydroxyl, C₁₋₄ alkyl or C₁₋₄ alkoxy. In some embodiments Y is —C(CH₃)₂CH₂C(O)— or —C(O)CH₂C(CH₃)₂— optionally substituted with halogen, hydroxyl, C₁₋₄ alkyl or C₁₋₄ alkoxy. In some embodiments Y is —CH₂CH₂C(O)— or —C(O)CH₂CH₂—. In some embodiments, compounds of the present invention can be represented by Formulae (IIIg) and (IIIh) respectively as illustrated below: 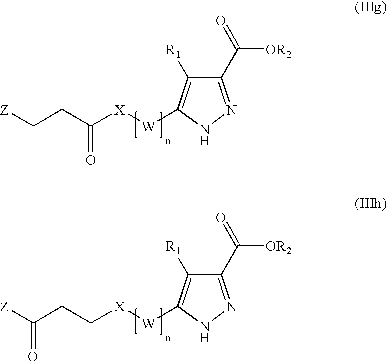
wherein each variable in Formulae (IIIg) and (IIIh) has the same meaning as described herein, supra and infra.

Some embodiments of the present invention pertain to compounds of Formula (I) wherein Y is —CH₂C(O)CH₂— optionally substituted with halogen, hydroxyl, C₁₋₄ alkyl or C₁₋₄ alkoxy. In some embodiments Y is —CH₂C(O)CH₂—. In some embodiments, compounds of the present invention can be represented by Formula (IIIi) as illustrated below: 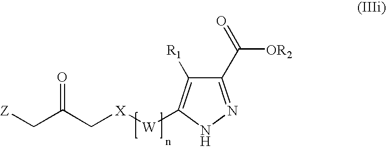
wherein each variable in Formula (IIIi) has the same meaning as described herein, supra and infra.

Some embodiments of the present invention pertain to compounds of Formula (I) wherein Y is —CH₂CH₂CH₂C(O)— or —C(O)CH₂CH₂CH₂— optionally substituted with halogen, hydroxyl, C₁₋₄ alkyl or C₁₋₄ alkoxy. In some embodiments Y is —CH(CH₃)CH₂CH₂C(O)— or —C(O)CH₂CH₂CH(CH₃)— optionally substituted with halogen, hydroxyl, C₁₋₄ allyl or C₁₋₄ alkoxy. In some embodiments Y is —CH(CH₃)CH₂CH₂C(O)— or —C(O)CH₂CH₂CH(CH₃)—. In some embodiments, compounds of the present invention can be represented by Formulae (IIIj) and (IIIk) respectively as illustrated below: 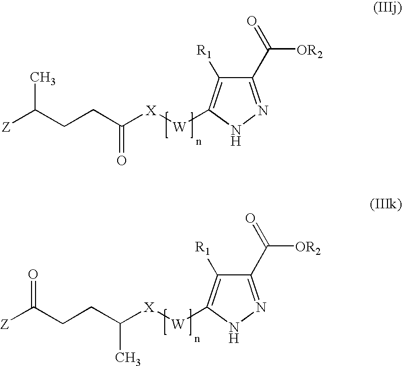
wherein each variable in Formulae (IIIj) and (IIIk) has the same meaning as described herein, supra and infra. In some embodiments Y is —CH₂CH₂CH₂C(O)— or —C(O)CH₂CH₂CH₂—. In some embodiments, compounds of the present invention can be represented by Formulae (IIIm) and (IIIn) respectively as illustrated below: 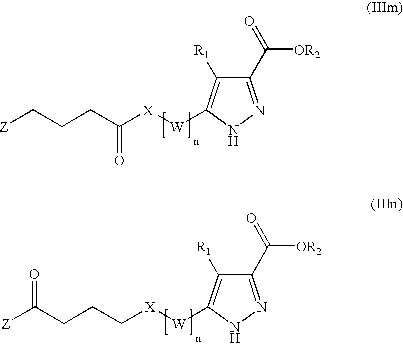
wherein each variable in Formulae (IIIm) and (IIn) has the same meaning as described herein, supra and infra.

Some embodiments of the present invention pertain to compounds of Formula (I) wherein Y is —CH₂CH₂C(O)CH₂— or —CH₂C(O)CH₂CH₂— optionally substituted with halogen, hydroxyl, C₁₋₄ alkyl or C₁₋₄ alkoxy. In some embodiments Y is —CH₂CH₂C(O)CH₂— or —CH₂C(O)CH₂CH₂—. In some embodiments, compounds of the present invention can be represented by Formulae (IIIo) and (IIIp) respectively as illustrated below: 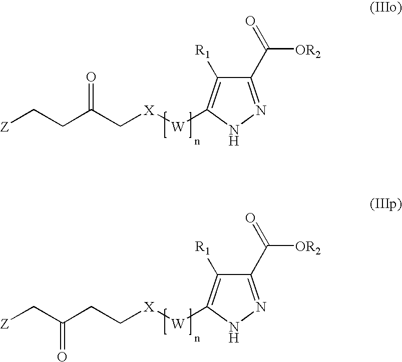
wherein each variable in Formulae (IIIo) and (IIIp) has the same meaning as described herein, supra and infra.

Some embodiments of the present invention pertain to compounds of Formula (I) wherein Y is —CH═CHC(O)— or —C(O)CH═CH— optionally substituted with halogen, hydroxyl, C₁₋₄ allyl or C₁₋₄ alkoxy. In some embodiments Y is —CH=CHC(O)— or —C(O)CH═CH—. In some embodiments, compounds of the present invention can be represented by Formulae (IIIq) and (IIIr) respectively as illustrated below: 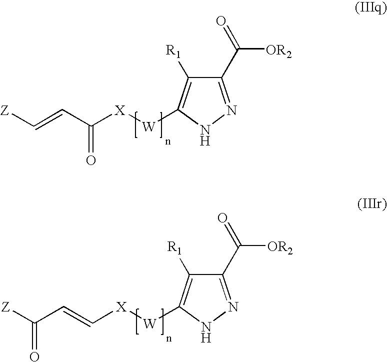
wherein each variable in Formulae (IIIq) and (IIIr) has the same meaning as described herein, supra and infra.

Some embodiments of the present invention pertain to compounds of Formula (I) wherein Y is —C(CH₃)═CHC(O)— or —C(O)CH═C(CH₃)— optionally substituted with halogen, hydroxyl, C₁₋₄ alkyl or C₁₋₄ alkoxy. In some embodiments Y is —C(CH₃)═CHC(O)— or —C(O)CH═C(CH₃)—. In some embodiments, compounds of the present invention can be represented by Formulae (IIIs) and (IIIt) respectively as illustrated below: 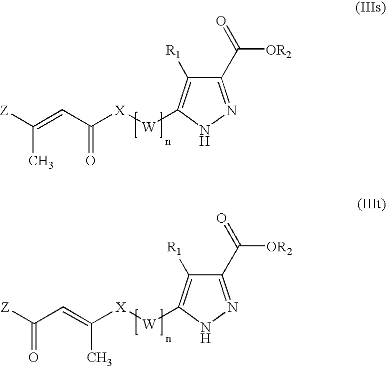
wherein each variable in Formulae (IIIs) and (IIIt) has the same meaning as described herein, supra and infra.

Some embodiments of the present invention pertain to compounds of Formula (I) wherein X is —NR₃C(O)—. In some embodiments, compounds of the present invention can be represented by Formula (IVa) as illustrated below: 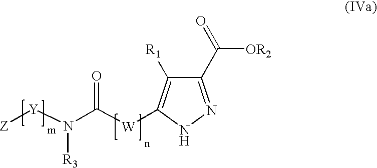
wherein each variable in Formula (IVa) has the same meaning as described herein, supra and infra.

Some embodiments of the present invention pertain to compounds of Formula (I) wherein X is —C(O)NR₃—. In some embodiments, compounds of the present invention can be represented by Formula (IVb) as illustrated below: 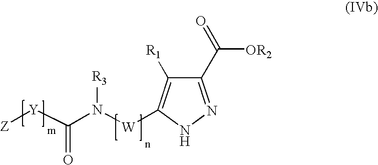
wherein each variable in Formula (IVb) has the same meaning as described herein, supra and infra.

Some embodiments of the present invention pertain to compounds of Formula (I) wherein X is —NR₃S(O)₂—. In some embodiments, compounds of the present invention can be represented by Formula (IVc) as illustrated below: 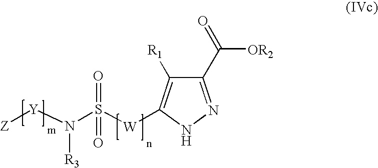
wherein each variable in Formula (IVc) has the same meaning as described herein, supra and infra.

Some embodiments of the present invention pertain to compounds of Formula (I) wherein X is —S(O)ₓNR₃—. In some embodiments, compounds of the present invention can be represented by Formula (IVd) as illustrated below: 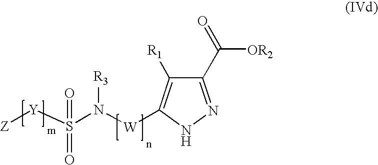
wherein each variable in Formula (IVd) has the same meaning as described herein, supra and infra.

Some embodiments of the present invention pertain to compounds of Formula (I) wherein X is —NR₃C(O)NR₄—. In some embodiments, compounds of the present invention can be represented by Formula (IVe) as illustrated below: 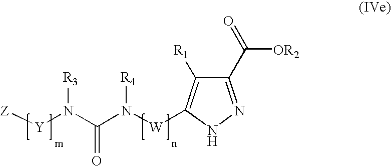
wherein each variable in Formula (IVe) has the same meaning as described herein, supra and infra.

Some embodiments of the present invention pertain to compounds of Formula (I) wherein X is —NR₃C(O)O—. In some embodiments, compounds of the present invention can be represented by Formula (IVf) as illustrated below: 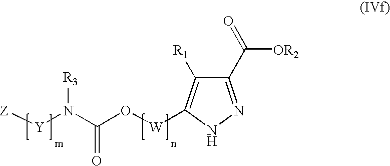
wherein each variable in Formula (IVf) has the same meaning as described herein, supra and infra.

Some embodiments of the present invention pertain to compounds of Formula (I) wherein X is —OC(O)NR₃—. In some embodiments, compounds of the present invention can be represented by Formula (IVg) as illustrated below: 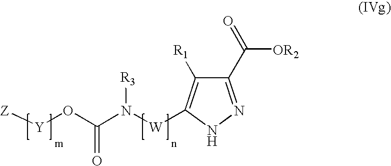
wherein each variable in Formula (IVg) has the same meaning as described herein, supra and infra.

Some embodiments of the present invention pertain to compounds of Formula (I) wherein X is —NR₃—. In some embodiments, compounds of the present invention can be represented by Formula (IVh) as illustrated below: 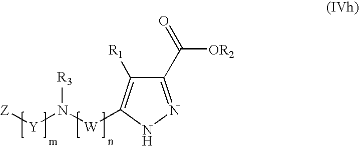
wherein each variable in Formula (IVh) has the same meaning as described herein, supra and infra.

Some embodiments of the present invention pertain to compounds of Formula (I) wherein R₃ is H or CH₃.

Some embodiments of the present invention pertain to compounds of Formula (I) wherein R₄ is H or CH₃.

Some embodiments of the present invention pertain to compounds of Formula (I) wherein X is —C(O)—. In some embodiments, compounds of the present invention can be represented by Formula (IVi) as illustrated below: 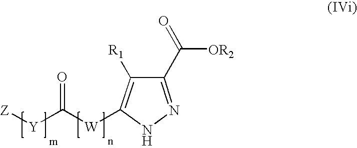
wherein each variable in Formula (IVi) has the same meaning as described herein, supra and infra.

Some embodiments of the present invention pertain to compounds of Formula (I) wherein X is —CH(OH)—. In some embodiments, compounds of the present invention can be represented by Formula (IVj) as illustrated below: 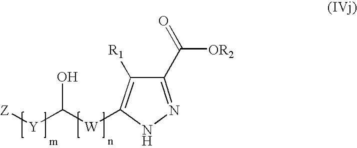
wherein each variable in Formula (IVj) has the same meaning as described herein, supra and infra.

Some embodiments of the present invention pertain to compounds of Formula (I) wherein X is —C(NH)—. In some embodiments, compounds of the present invention can be represented by Formula (IVk) as illustrated below: 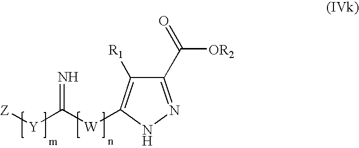
wherein each variable in Formula (IVk) has the same meaning as described herein, supra and infra.

Some embodiments of the present invention pertain to compounds of Formula (I) wherein X is —O—. In some embodiments, compounds of the present invention can be represented by Formula (IVl) as illustrated below: 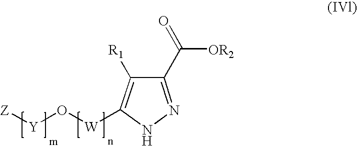
wherein each variable in Formula (IVl) has the same meaning as described herein, supra and infra.

Some embodiments of the present invention pertain to compounds of Formula (I) wherein X is —S—. In some embodiments, compounds of the present invention can be represented by Formula (IVm) as illustrated below: 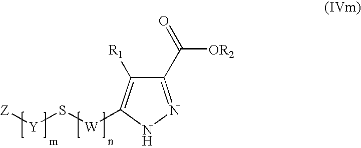
wherein each variable in Formula (IVm) has the same meaning as described herein, supra and infra.

Some embodiments of the present invention pertain to compounds of Formula (I) wherein X is —S(O)—. In some embodiments, compounds of the present invention can be represented by Formula (IVn) as illustrated below: 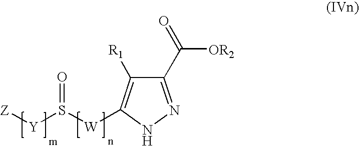
wherein each variable in Formula (IVn) has the same meaning as described herein, supra and infra.

Some embodiments of the present invention pertain to compounds of Formula (I) wherein X is —S(O)₂—. In some embodiments, compounds of the present invention can be represented by Formula (IVo) as illustrated below: 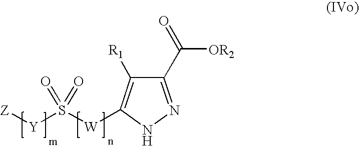
wherein each variable in Formula (IVo) has the same meaning as described herein, supra and infra.

Some embodiments of the present invention pertain to compounds of Formula (I) wherein Z is H.

Some embodiments of the present invention pertain to compounds of Formula (I) wherein Z is halogen.

Some embodiments of the present invention pertain to compounds of Formula (I) wherein Z is phenyl. In some embodiments the phenyl is optionally substituted with 1 to 5 substituents selected from the group consisting of halogen, C₁₋₄ haloalkyl, C₁₋₄ alkylamino, di-C₁₋₄-alkylamino, C₁₋₄ alkyl, C₁₋₄ alkoxy, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl. In some embodiments the phenyl is optionally substituted with 1 to 3 substituents selected from the group consisting of —F, —Cl, —Br, —CF₃, —NHCH₃, —N(CH₃)₂, —CH₃, —CH₂CH₃, —OCH₃ and —OCF₃.

Some embodiments of the present invention pertain to compounds of Formula (I) wherein Z is heteroaryl. In some embodiments the heteroaryl is optionally substituted with 1 to 5 substituents selected from the group consisting of halogen, C₁₋₄ haloalkyl, C₁₋₄ alkylamino, di-C₁₋₄-alkylamino, C₁₋₄ alkyl, C₁₋₄ alkoxy, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl. In some embodiments the phenyl is optionally substituted with 1 to 3 substituents selected from the group consisting of —F, —Cl, —Br, —CF₃, —NHCH₃, —N(CH₃)₂, —CH₃, —CH₂CH₃, —OCH₃ and —OCF₃.

Some embodiments of the present invention pertain to compounds of Formula (I) wherein R₁ is H. In some embodiments, compounds of the present invention can be represented by Formula (IVp) as illustrated below: 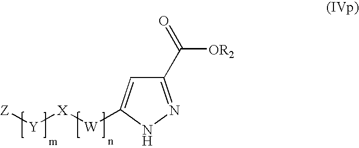
wherein each variable in Formula (IVp) has the same meaning as described herein, supra and infra.

Some embodiments of the present invention pertain to compounds of Formula (I) wherein R₁ is hydroxyl. In some embodiments, compounds of the present invention can be represented by Formula (IVq) as illustrated below: 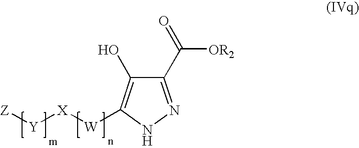
wherein each variable in Formula (IVq) has the same meaning as described herein, supra and infra.

Some embodiments of the present invention pertain to compounds of Formula (I) wherein R₁ is halogen. In some embodiments R₁ is F, Cl or Br. In still further embodiments, R₁ is F (a fluorine atom). In some embodiments, compounds of the present invention can be represented by Formula (IVr) as illustrated below: 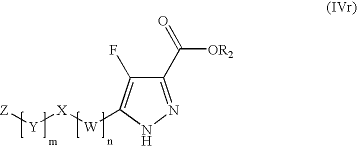
wherein each variable in Formula (IVr) has the same meaning as described herein, supra and infra.

Some embodiments of the present invention pertain to compounds of Formula (I) wherein R₁ is C₁₋₄ alkyl.

Some embodiments of the present invention pertain to compounds of Formula (I) wherein R₁ is C₁₋₄ haloalkyl.

Some embodiments of the present invention pertain to compounds of Formula (I) wherein R₂ is H. In some embodiments, compounds of the present invention can be represented by Formula (IVs) as illustrated below: 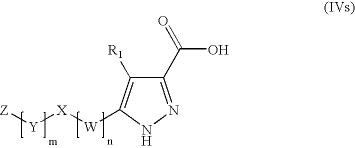
wherein each variable in Formula (IVs) has the same meaning as described herein, supra and infra.

Some embodiments of the present invention pertain to compounds of Formula (I) wherein R₂ is C₁₋₈ alkyl.

### CHEMISTRY OF THE PRESENT INVENTION

Tautomers

Although compounds of the present invention of Formula (I) are depicted as one compound, it is well understood and appreciated in the art that pyrazoles can exist in various tautomeric forms. Two possible tautomeric forms are illustrated below: 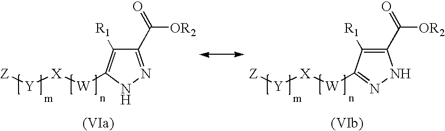
Accordingly, tautomeric forms can have corresponding nomenclature, for example, Formula (VIa) and Formula (VIb) can be represented by the general chemical names 1H-pyrazole and 2H-pyrazole respectively. Therefore, for convenience, compounds presented herein by Formula (I) are understood to include all tautomers and furthermore, these tautomers and various nomenclature designations are within the scope of the present invention.
Enantiomers, Diastereomers and Mixtures Thereof:

Compounds of Formula (I) may have one or more chiral centers, and therefore exist as enantiomers or diastereomers. The invention is understood to extend to all such enantiomers, diastereomers and mixtures thereof, including racemates. Formula (I) and the formulae described herein, supra, are intended to represent all individual isomers and mixtures thereof, unless stated or shown otherwise.

Racemic mixtures can be resolved into the optical pure enatiomers by known methods, for example, by separation of diastereomeric salts thereof with an optically active acid, and liberating the optically active amine compound by treatment with a base. Another method for resolving racemates into the optical pure enatiomers is based upon chromatography on an optically active matrix or chiral support. Certain racemic compounds of the present invention can thus be resolved into their optical antipodes, e.g., by fractional crystallization of d- or 1-(tartrates, mandelates, or camphorsulphonate) salts for example. The compounds of the present invention may also be resolved by the formation of diastereomeric amides or ester by reaction of the compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the compounds of the present invention with an optically active chloroformate or the like subsequently hydrolyzed.

Additional methods for the resolution of optical isomers, known to those skilled in the art can be used, and will be apparent to the average worker skilled in the art. Such methods include those discussed by J. Jaques, A. Collet, and S. Wilen in “Enantiomers, Racemates, and Resolutions”, John Wiley and Sons, New York (1981).

Synthesis of Compounds of Formula (I)

The compounds of the present invention can be readily prepared according to a variety of synthetic regimes, all of which would be familiar to one skilled in the art. The chemical and patent literature quotes numerous procedures for the synthesis of pyrazole carboxylic acids and esters. Some of these articles include: Ashton and co-workers, J. Med. Chem. 1993, 36, 3595-3605; Seki and co-workers, Chem. Pharm. Bull., 1984, 32, 1568; and Wiley and Hexner, Org. Syn Coll IV, 1963, 351.

Also provided is a novel procedure for the preparation of novel pyrazoles of Formula (I).

In the illustrated syntheses outlined below, the labeled substituents have the same identifications as set out in the definitions of the compound described above for Formula (I). The methods described below can be used for the preparation of compounds of the invention.

One method that can be used to prepare compounds of Formula (I) utilizes intermediates of Formula (A) as illustrated in Reaction Scheme (1) below:

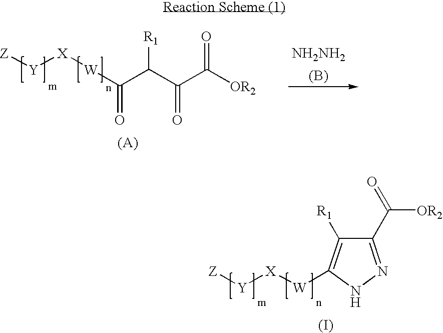

Compounds of Formula (I) can be prepared by treating the 2,4-diketo ester or acid [R₂=alkyl or H respectively, (A)] with hydrazine (B) under various conditions. For example, the solvent may optionally be present or absent. In the instance that the solvent is absent then the hydrazine (B) serves both as a reactant and as the solvent. Typically, under these conditions hydrazine would be present in molar excess. In the instance when the solvent is present, the solvent can be a polar solvent and is generally a C₁-C₆ alcohol. Some typical solvents can be selected from, but not limited to, the group consisting of methanol, ethanol, butanol, pentanol, hexanol, 2-methoxyethanol, 1-propanol and 2-propanol. In some instances it can be beneficial to include the presence of an acid. Some representative examples of acids that can be used can be selected from the group consisting of hydrochloric acid, hydrobromic acid, acetic acid and trifluoroacetic acid. The reaction temperature generally ranges from about 20° C. to about 160° C., and for convenience, the reaction temperature is typically the reflux temperature of the reaction mixture.

The 2,4-diketo esters or acids (A) are commercially available or can be obtained by methods known in the art, Seki and co-workers, Chem. Pharm. Bull., 1984, 32, 1568. It is appreciated that a group on the Z[Y]ₘ—X—[W]ₙ— chain of (A) can be protected by methods known in the art if such protection is required.

One particular feature of 2,4-diketo esters or acids (A) is that a diverse number of R₁ groups can be introduced by a variety of methods known in the art, such as, alkylation, as shown in Reaction Scheme (2) below: 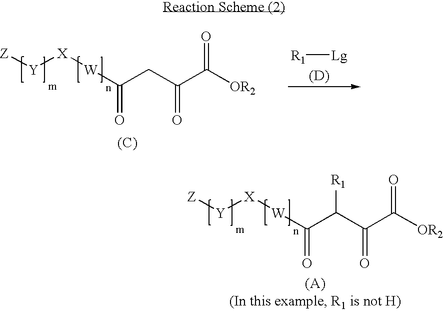
The alkylation step as shown in Reaction Scheme (2) is similar to and in some instances identical to that described in the preparation of intermediate (F), infra.

Utilizing a similar starting material as in Reaction Scheme (1) an alternative method can be used to prepare compounds of Formula (I) as illustrated in Reaction Scheme (3) below:

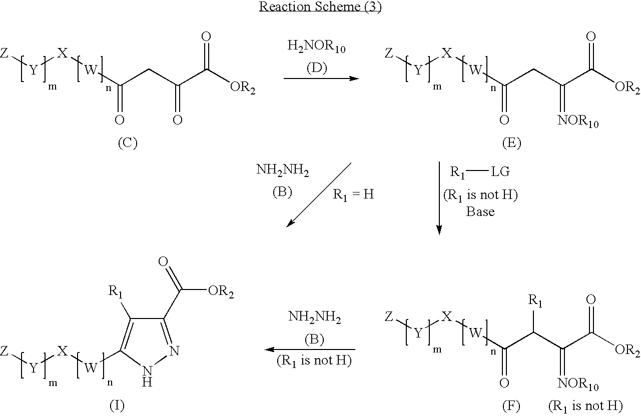

Compounds of Formula (I) can be prepared by treating 2,4diketo ester or acid (C), preferably the ester, with an alkoxyamine of Formula (D) where R₁₀ is C₁-C₈ alkyl, leading to 2-(methoxyimino) intermediate of Formula (E). Typically, the alkoxyamine is methoxyamine (i.e., O-methyl hydroxylamine) wherein R₁₀ is methyl. This step is typically conducted in the presence of a drying agent to concomitantly remove the water formed during the process; examples of a drying agent that can be used include molecular sieves, magnesium sulfate and the like. In the subsequent step, the intermediate of Formula (E) can be functionalized with R₁ utilizing methods known in the art. One example may use R₁-LG, wherein LG is a leaving group, such as, iodo, bromo, mesylate and the like, in the presence of a base and a polar solvent. Typical bases can be selected from, potassium carbonate, sodium carbonate, sodium hydroxide, potassium hydroxide, lithium hydroxide, LDA, sodium methoxide, sodium ethoxide and the like; and the polar solvent can be dimethylformamide, dimethylsulfoxide, THF and the like. It is understood that this step is optional since in some embodiments of the invention R₁ is H. In this context, as depicted in Reaction Scheme (3), intermediate (E) can be converted to a compound of Formula (I) wherein R₂ is H using hydrazine (B). This step can be performed under heating conditions in an alcoholic solvent as described above in Reaction Scheme (1). Optionally, an acid can be present, such as HCl. Similarly, in the example where R₁ is not H, intermediate (F) can be treated with hydrazine (B) in a manner as described above to provide compounds of Formula (I) where R₁ is a group other than H.

It is understood that in reference to Reaction Scheme (3), a compound of Formula (C) can be functionalized with R₁ as described above prior to treating with alkoxyamine (D) to give the same intermediate (F). Absent any chemical reason that would be known in the art, the order of the steps can be changed and can be more a matter of convenience than necessity [i.e., (C) to (E) to (F); or (C) to (A) to (F)].

A novel method for the preparation of compounds of Formula (I) is shown in Reaction Scheme (4). 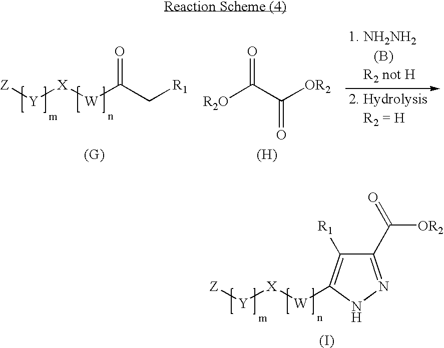
In this procedure, ketone (G) is allowed to react with a base, such as sodium methoxide or other alkali metal alkoxide, in the presence of oxalate (H). The resulting mixture is treated with hydrazine (B) to compounds of the present invention wherein R₂ is alkyl. As an optional step, the ester can be converted to the carboxylic acid by methods known in the art.

Another method for the preparation of compounds of Formula (I) is set forth in Reaction Scheme (5) and is intended to be illustrative and not limited.

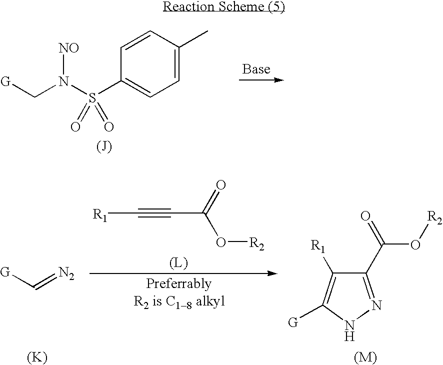

Compounds of Formula (I) can be prepared by treating a nitroso sulfonamide (L) with base to give a substituted diazoalkane (K). A wide variety of functionality can be present in this synthesis, for example, G can be (CH₃O)₂CH— as shown in Example infra. Once formed, the substituted diazoalkane (K) can undergo a cycloaddition process with alkyne (L) to give pyrazole (M). A variety of alkynes can be either prepared or purchased from commercial sources to introduce the R₁ group as defined herein. Preferrably, R₁ in Reaction Scheme (5) is H, C₁₋₄ alkyl or C₁₋₄ haloalkyl.

It is understood that various pyrazoles can be prepared with groups present at the 5-position and these groups can be further converted or modified using methods known in the art into compounds of Formula (I).

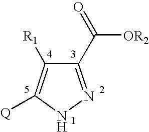

For example, Q can be a group represented by formula H₂N—[W]ₙ—, wherein “W” and “n” have the same meaning as used herein. The amine group can be modified with a variety of substituted aldehydes or ketones, such as those commercially available or prepared by methods known in the art, through a reductive amination procedure or similar method. Further, the amine can also be alkylated with Z[Y]ₘ-Lg wherein Lg is a leaving group as defined herein, supra. In addition, the amine can also be modified with a variety of electrophils, such as Z-[Y]ₘ—C(O)-Lg (i.e., an acid halide or anhydride), Z-[Y]ₘ—S(O)₂-Lg (i.e., a sulfonyl halide) or Z-[Y]ₘ—N═C═O. In the examples where R₃ is not H, the amine can be further substituted with the R₃ group via methods known in the art. In another example, Q can be HX—[W]ₙ— wherein X is “—O—” or “—S—” and can be modified via methods known in the art to give compounds of Formula (I), for example, via an alkylation procedure with Z-[Y]ₘ-Lg wherein Lg is a leaving group defined herein, supra or via the Mitsunobu reaction. In the scenario where X is “—S—”, conversion can to the corresponding sulfoxide [i.e., X═—S(O)—] or sulfone [i.e., X═—S(O)₂—] can be implemented via oxidation, for example, mCPPA or H₂O₂. It is understood that the chemistry described for the Q group can be “reversed” or used in an “alternative” manner with the corresponding reactant. To illustrate this point, the aldehyde or ketone group can alternatively be part of the Q group and modified with a variety of amines using the similar synthetic procedures described above, such as, reductive amination (see Examples, infra). Likewise, in an alternative manner, Q can be Lg-[W]ₙ— and used, for example, to alkylate Z-[Y]ₘ—XH.

Esters of the compounds shown herein, wherein R₂=alkyl, can be readily converted to the corresponding carboxylic acids of Formula (I) by methods known in the art, such as alkaline hydrolysis using LiOH, NaOH, KOH, and the like. Another method for the conversion of an ester to a carboxylic acid of Formula (I) is through the use of acid hydrolysis, such as aqueous HCl and the like. Generally, the solvent is an aqueous mixture with a polar solvent as described above.

The various organic group transformations and protecting groups utilized herein can be performed by a number of procedures other than those described above. References for other synthetic procedures that can be utililized for the preparation of intermediates or compounds disclosed herein can be found in, for example, Smith, M. B.; and March, J., Advanced Organic Chgemistry, 5^{th} Edition, Wiley-Interscience (2001); Larock, R. C., Comprehensive Organic Transformations, A Guide to Functional Group Preparations, 2^{nd} Edition, VCH Publishers, Inc. (1999), or Wuts, P. G. M.; Greene, T. W.; Protective Groups in Organic Synthesis, 3^{rd} Edition, John Wiley and Sons, (1999), all three incorporated in their entirety herein by reference.

It is understood that the chemistry described here is representative and is not intended to be limiting in any manner.

Representative examples of compound of Formula (I) are shown below in TABLE A.

TABLE A

| Cmpd # | Structure | Chemical Name |
|---|---|---|
| 1 | 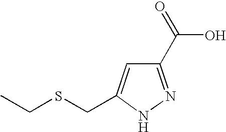 | 5-Ethylsulfanylmethyl-1H-py- razole-3-carboxylic acid |
| 2 | 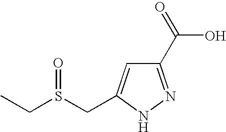 | 5-Ethanesulfinylmethyl-1H-py- razole-3-carboxylic acid |
| 3 | 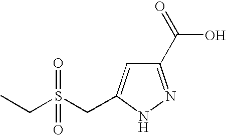 | 5-Ethanesulfonylmethyl-1H-py- razole-3-carboxylic acid |
| 4 | 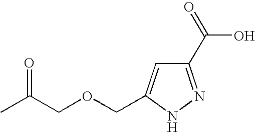 | 5-(2-Oxo-propoxymethyl)-1H-py- razole-3-carboxylic acid |
| 5 | 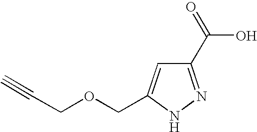 | 5-Prop-2-ynyloxymethyl-1H-py- razole-3-carboxylic acid |
| 6 | 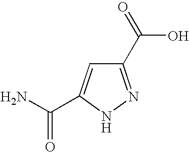 | 5-Carbamoyl-1H-pyrazole-3-car- boxylic acid |
| 7 | 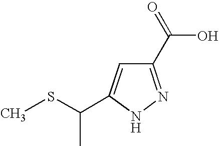 | 5-(1-Methylsulfanyl-ethyl)-1H-py- razole-3-carboxylic acid |
| 8 | 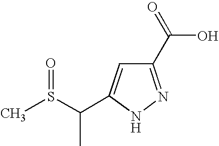 | 5-(1-Methanesulfinyl-ethyl)-1H-py- razole-3-carboxylic acid |
| 9 | 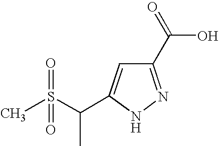 | 5-(1-Methanesulfonyl-ethyl)-1H-py- razole-3-carboxylic acid |
| 10 | 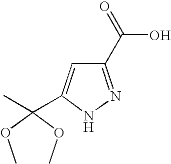 | 5-(1,1-Dimethoxy-ethyl)-1H-py- razole-3-carboxylic acid |
| 11 | 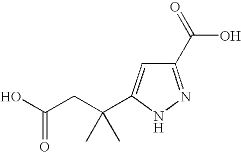 | 5-(2-Carboxy-1,1-dimethyl- ethyl)-1H-pyrazole-3-car- boxylic acid |
| 12 | 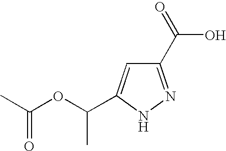 | 5-(1-Acetoxy-ethyl)-1H-py- razole-3-carboxylic acid |
| 13 | 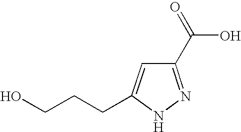 | 5-(3-Hydroxy-propyl)-1H-py- razole-3-carboxylic acid |
| 14 | 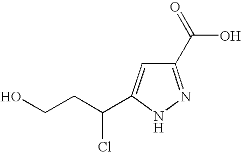 | 5-(1-Chloro-3-hydroxy- propyl)-1H-pyrazole-3-car- boxylic acid |
| 15 | 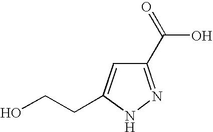 | 5-(2-Hydroxy-ethyl)-1H-py- razole-3-carboxylic acid |
| 16 | 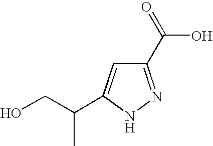 | 5-(2-Hydroxy-1-methyl-ethyl)-1H-py- razole-3-carboxylic acid |
| 17 | 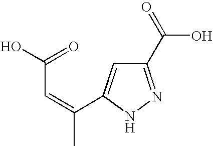 | 5-(2-Carboxy-1-methyl-vinyl)-1H-py- razole-3-carboxylic acid |
| 18 | 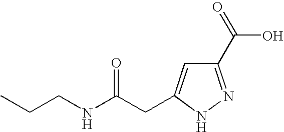 | 5-Propylcarbamoylmethyl-1H-py- razole-3-carboxylic acid |
| 19 | 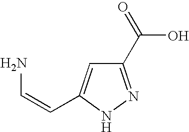 | 5-(2-Amino-vinyl)-1H-py- razole-3-carboxylic acid |
| 20 | 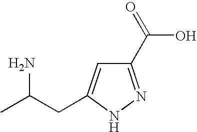 | 5-(2-Amino-propyl)-1H-py- razole-3-carboxylic acid |
| 21 | 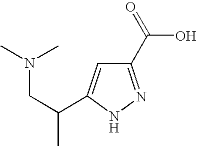 | 5-(2-Dimethylamino-1-methyl- ethyl)-1H-pyrazole-3-car- boxylic acid |
| 22 | 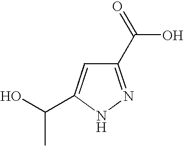 | 5-(1-Hydroxy-ethyl)-1H-py- razole-3-carboxylic acid |
| 23 | 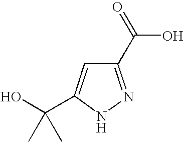 | 5-(1-Hydroxy-1-methyl-ethyl)-1H-py- razole-3-carboxylic acid |
| 24 | 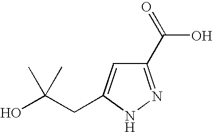 | 5-(2-Hydroxy-2-methyl- propyl)-1H-pyrazole-3-car- boxylic acid |
| 25 | 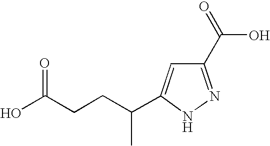 | 5-(3-Carboxy-1-methyl- propyl)-1H-pyrazole-3-car- boxylic acid |
| 26 | 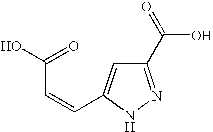 | 5-(2-Carboxy-vinyl)-1H-py- razole-3-carboxylic acid |
| 27 | 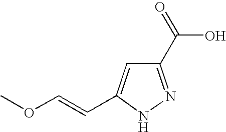 | 5-(2-Methoxy-vinyl)-1H-py- razole-3-carboxylic acid |
| 28 | 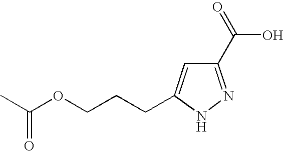 | 5-(3-Acetoxy-propyl)-1H-py- razole-3-carboxylic acid |
| 29 | 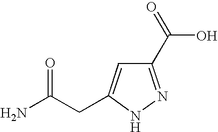 | 5-Carbamoylmethyl-1H-py- razole-3-carboxylic acid |
| 30 | 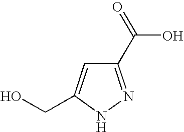 | 5-Hydroxymethyl-1H-py- razole-3-carboxylic acid |
| 31 | 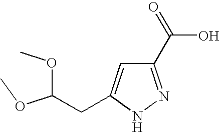 | 5-(2,2-Dimethoxy-ethyl)-1H-py- razole-3-carboxylic acid |
| 32 | 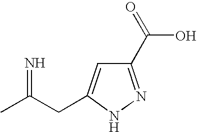 | 5-(2-Imino-propyl)-1H-py- razole-3-carboxylic acid |
| 33 | 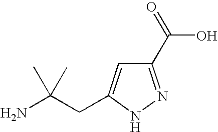 | 5-(2-Amino-2-methyl-propyl)-1H-py- razole-3-carboxylic acid |
| 34 | 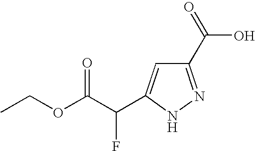 | 5-(Ethoxycarbonyl-fluoro- methyl)-1H-pyrazole-3-car- boxylic acid |
| 35 | 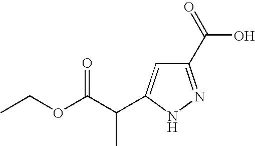 | 5-(1-Ethoxycarbonyl-ethyl)-1H-py- razole-3-carboxylic acid |
| 36 | 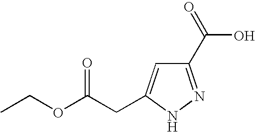 | 5-Ethoxycarbonylmethyl-1H-py- razole-3-carboxylic acid |
| 37 | 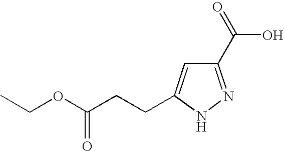 | 5-(2-Ethoxycarbonyl-ethyl)-1H-py- razole-3-carboxylic acid |
| 38 | 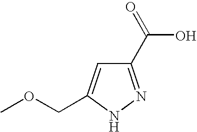 | 5-Methoxymethyl-1H-py- razole-3-carboxylic acid |
| 39 | 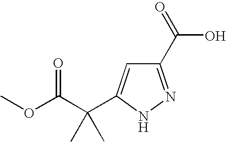 | 5-(1-Methoxycarbonyl-1-meth- yl-ethyl)-1H-pyrazole-3-car- boxylic acid |
| 40 | 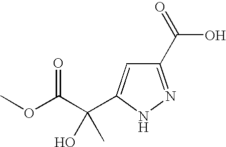 | 5-(1-Hydroxy-1-meth- oxycarbonyl-ethyl)-1H-py- razole-3-carboxylic acid |
| 41 | 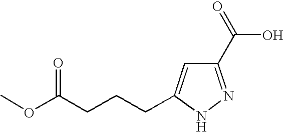 | 5-(3-Methoxycarbonyl- propyl)-1H-pyrazole-3-car- boxylic acid |
| 42 | 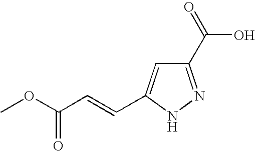 | 5-(2-Methoxycarbonyl-vinyl)-1H-py- razole-3-carboxylic acid |
| 43 | 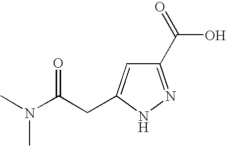 | 5-Dimethylcarbamoylmethyl-1H-py- razole-3-carboxylic acid |
| 44 | 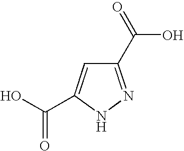 | 1H-Pyrazole-3,5-dicarboxylic acid |
| 45 | 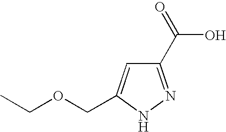 | 5-Ethoxymethyl-1H-pyrazole-3-car- boxylic acid |
| 46 | 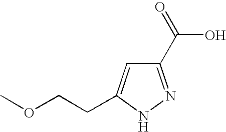 | 5-(2-Methoxy-ethyl)-1H-py- razole-3-carboxylic acid |
| 47 | 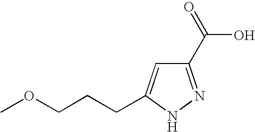 | 5-(3-Methoxy-propyl)-1H-py- razole-3-carboxylic acid |
| 48 | 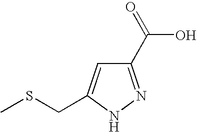 | 5-Methylsulfanylmethyl-1H-py- razole-3-carboxylic acid |
| 49 | 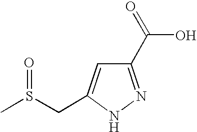 | 5-Methanesulfinylmethyl-1H-py- razole-3-carboxylic acid |
| 50 | 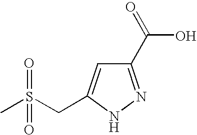 | 5-Methanesulfonylmethyl-1H-py- razole-3-carboxylic acid |
| 51 | 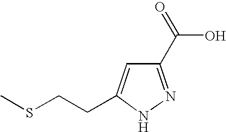 | 5-(2-Methylsulfanyl-ethyl)-1H-py- razole-3-carboxylic acid |
| 52 | 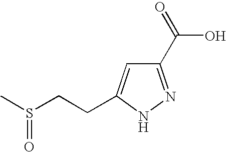 | 5-(2-Methanesulfinyl-ethyl)-1H-py- razole-3-carboxylic acid |
| 53 | 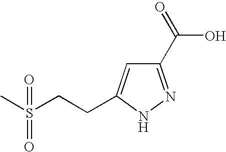 | 5-(2-Methanesulfonyl-ethyl)-1H-py- razole-3-carboxylic acid |
| 54 | 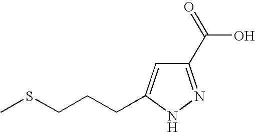 | 5-(3-Methylsulfanyl-propyl)-1H-py- razole-3-carboxylic acid |
| 55 | 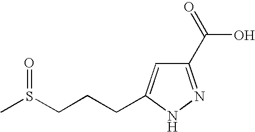 | 5-(3-Methanesulfinyl-propyl)-1H-py- razole-3-carboxylic acid |
| 56 | 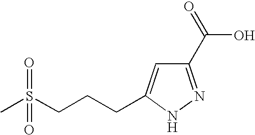 | 5-(3-Methanesulfonyl-propyl)-1H-py- razole-3-carboxylic acid |
| 57 | 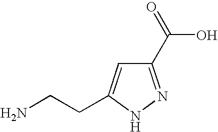 | 5-(2-Amino-ethyl)-1H-py- razole-3-carboxylic acid |
| 58 | 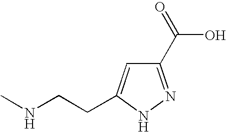 | 5-(2-Methylamino-ethyl)-1H-py- razole-3-carboxylic acid |
| 59 | 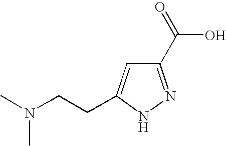 | 5-(2-Dimethylamino-ethyl)-1H-py- razole-3-carboxylic acid |
| 60 | 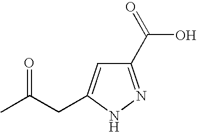 | 5-(2-Oxo-propyl)-1H-py- razole-3-carboxylic acid |
| 61 | 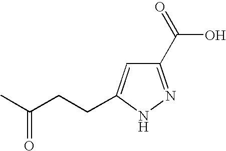 | 5-(3-Oxo-butyl)-1H-pyrazole-3-car- boxylic acid |
| 62 | 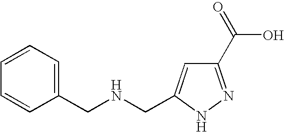 | 5-(Benzylamino-methyl)-1H-py- razole-3-carboxylic acid |
| 63 | 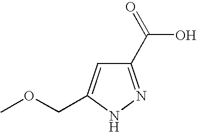 | 5-Methoxymethyl-1H-py- razole-3-carboxylic acid |
| 64 | 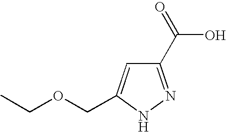 | 5-Ethoxymethyl-1H-pyrazole-3-car- boxylic acid |
| 65 | 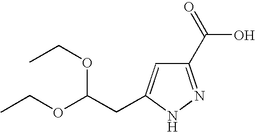 | 5-(2,2-Diethoxy-ethyl)-1H-py- razole-3-carboxylic acid |

It is understood that the present invention includes compounds shown in TABLE A and corresponding tautomers and esters thereof.

### COMPOSITIONS OF THE PRESENT INVENTION

Some embodiments of the present invention include a method of producing a pharmaceutical composition comprising admixing at least one compound according to any of the compound embodiments disclosed herein and a pharmaceutically acceptable carrier.

Formulations can be prepared by any suitable method, typically by uniformly mixing the active compound(s) with liquids or finely divided solid carriers, or both, in the required proportions, and then, if necessary, forming the resulting mixture into a desired shape.

Conventional excipients, such as binding agents, fillers, acceptable wetting agents, tabletting lubricants, and disintegrants can be used in tablets and capsules for oral administration. Liquid preparations for oral administration can be in the form of solutions, emulsions, aqueous or oily suspensions, and syrups. Alternatively, the oral preparations can be in the form of dry powder that can be reconstituted with water or another suitable liquid vehicle before use. Additional additives such as suspending or emulsifying agents, non-aqueous vehicles (including edible oils), preservatives, and flavorings and colorants can be added to the liquid preparations. Parenteral dosage forms can be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro, A. R., et al.).

While it is possible that a compound for use in the prophylaxis or treatment of the present invention may, in an alternative use, be administered as a raw or pure chemical, it is preferable however to present the compound or “active ingredient” as a pharmaceutical formulation or composition further comprising a pharmaceutically acceptable carrier. Therefore, one aspect of the present invention encompasses pharmaceutical compositions comprising a pharmaceutically acceptable carrier in combination with at least one compound according to Formula (I):

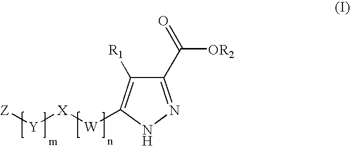

wherein:

W and Y are independently a straight or branched chain C₁₋₅ alkylene group optionally containing one double bond, one triple bond or carbonyl, wherein said C₁₋₅ alkylene group is optionally substituted with halogen, hydroxyl, C₁₋₄ alkyl, C₁₋₅ haloalkyl or C₁₋₄ alkoxy;

X is —NR₃C(O)—, —C(O)NR₃, —NR₃S(O)₂—, —S(O)₂NR₃—, —NR₃C(O)NR₄—, —NR₃C(O)O—, —OC(O)NR₃—, —NR₃—, —C(O)—, —CH(OH)—, —C(NH)—, —O—, —S—, —S(O)— or —S(O)₂—;

R₃ and R₄ are independently H, C₁₋₄ alkyl, phenyl or heteroaryl, wherein each of the alkyl, phenyl and heteroaryl are optionally substituted with 1 to 5 substituents selected from the group consisting of halogen, hydroxyl, thiol, cyano, nitro, C₁₋₄ haloalkyl, amino, C₁₋₄ alkylamino, di-C₁₋₄-alkylamino, C₁₋₄ alkyl, C₁₋₄ alkoxy, C₂₋₄ alkenyl, C₂₋₄ alkynyl, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl;

Z is H, halogen, phenyl or heteroaryl, wherein said phenyl and heteroaryl are optionally substituted with 1 to 5 substituents selected from the group consisting of halogen, hydroxy, thiol, cyano, nitro, C₁₋₄ haloalkyl, amino, C₁₋₄ alkylamino, di-C₁₋₄-alkylamino, C₁₋₄ alkyl, C₁₋₄ alkoxy, C₂₋₄ alkenyl, C₂₋₄ alkynyl, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl;

R₁ is H, hydroxyl, halogen, C₁₋₄ allyl or C₁₋₄ haloalkyl;

R₂ is H or C₁₋₈ alkyl and

“n” and “m” are each independently 0 or 1; or

a pharmaceutically acceptable salt, solvate or hydrate thereof;

provided that when X is —NR₃— then “n” is 1.

Applicant reserves the right to exclude one or more of the compounds from any pharmaceutical composition embodiment; for example, one or more compounds can be excluded from any pharmaceutical composition embodiment selected from the group consisting of:
i) R₁ and R₂ are both H and —[W]ₙ—X—[Y]ₘ-Z together is selected from the group consisting of: CO₂H, C(O)—C₆H₄-p-O—C₈H₁₇, OCH₂CH₃, OH, CH₂CH₂CH₂CH₂CO₂H, CH₂CH₂CH₂CO₂H, CH₂CO₂H and CH₂CH₂CO₂H:
ii) R₁ is CH₃, R₂ is H and —[W]ₙ-X—[Y]ₘ-Z together is selected from the group consisting of CH₂CO₂H, C(O)CH═CH C₆H₅, C(O)C₆H₄-p-OCH₃, CO₂H, C(O)CH₃, C(O)C₆H₄-o-CH₃, C(O)C₆H₄-o-Br, C(O)C₆H₄-o-Cl, and C(O)C₆H₅;
iii) R₁ is Br, R₂ is H and —[W]ₙ—X—[Y]ₘ-Z together is CO₂H;
iv) R₁ is OH, R₂ is H and —[W]ₙ—X—[Y]ₘ-Z together is CO₂H;
v) R₁ is H, R₂ is CH₃ and —[W]ₙ—X—[Y]ₘ-Z together is selected from the group consisting of 2,6-dichloro-4-trifluoromethylphenoxy, C(O)NH—C₆H₄-p-OCH₂CH₃, NHC(O)CH(CH₃)₂, SCH₃, C(O)—C₆H-p-O—C₈H₁₇, SCH₂CH₃, C(O)NHC₆H₅, CH(OCH₃)₂, CH₂OC(O)CH₃, CO₂H, CO₂CH₃, C(O)C₆H₄-p-NO₂, C(O)C₆H₅, CH₂CH₂CO₂CH₃, CH₂CH₂CH₂CH₂CO₂CH₃, CH₂CH₂CH₂CO₂CH₃ and CH₂CO₂CH₃;
vi) R₁ is OH, R₂ is CH₃ and —[W]ₙ—X—[Y]ₘ-Z together is selected from the group consisting of CH₂OCH₂C₆H₅, CH₂OCH(CH₃)₂ and CH₂OH;
vii) R₂is CH₃:
   R₁ is CH₃ and —[W]ₙ—X—[Y]ₘ-Z together is 2,6-dichloro-4-trifluoromethylphenoxy;
   R₁ is I and —[W]ₙ—X—[Y]ₘ-Z together is CO₂C(CH₃)₃;
   R₁ is C(CH₃)₃ and —[W]ₙ—X—[Y]ₘ-Z together is formyl;
   R₁ is Br and —[W]ₙ—X—[Y]ₘ-Z together is CO₂CH₃; and
   R₁ is CH₂CH₂CH₂CH₃ and —[W]ₙ—X—[Y]ₘ-Z together is formyl;.
viii) R₁ is H; R₂ is CH₂CH₃ and —[W]ₓ—X—[Y]ₘ-Z together is selected from the group consisting of CH₂SCH₂CH₃, OCH₂CH₂CH═CH₂, CH₂CH₂CH₂OH, CH₂CH₂CHO, CO₂CH₂CH₃, OCH₃, C(O)CH₂Br, CO₂C₈H₁₇, formyl, OH, CH₂N(CH₂CH₂Cl)₂, CH(CH₃)OC(O)CH₃, CH₂OH, CH₂OC(O)CH₃, C(O)CH₃, C(O)C₆H₅ and C(O)NHCH₂CO₂CH₂CH₃.
ix) R₁ is CH₃, R₂ is CH₂CH₃ and —[W]ₙ—X—[Y]ₘ-Z together is selected from the group consisting of CH(OH)C₆H₄-p-N(CH₃)₂, C(O)CH₂C(O)CH₃, CO₂CH₂C₆H₅, CO₂CH₃, C(O)CH₂CH₂CH₃, C(O)CH₃, C(O)C₆H₄-p-OCH₃, C(O)C₆H₄-o-Br, C(O)C₆H₄-p-Cl, C(O)C₆H₄-o-Cl, C(O)CH₂C₆H₅ and C(O)C₆H₅;
x) R₂is CH₂CH₃:
   R₁ is I and —[W]ₙ—X—[Y]ₘ-Z together is CO₂CH₂CH₃;
   R₁ is CF₃ and —[W]ₙ—X—[Y]ₘ-Z together is CO₂CH₂CH₃; and
   R₁ is Br and —[W]ₙ—X—[Y]ₘ-Z together is CO₂CH₂CH₃;
xi) R₁ is OH, R₂ is CH₂CH₃ and —[W]ₙ—X—[Y]ₘ-Z together is selected from the group consisting of C(O)CH₅, C(O)NH₂ and CO₂CH₂CH₃;
xii) R₁ is H, R₂ is C(CH₃)₃ and —[W]ₙ—X—[Y]ₘ-Z together is selected from the group consisting of CO₂C(CH₃)₃, C(O)NHC(O)CH₃ and C(O)NH₂; and
xiii) R₁ is OH, R₂ is CH₂CH₂CH₂CH₃ and —[W]ₙ—X—[Y]ₘ-Z together is C(O)C₆H₅.

While it is possible that a compound of the invention may, in an alternative mode, be administered as a raw or pure chemical, it is preferable to present the compound or “active ingredient” as a pharmaceutical formulation or composition.

The invention provides pharmaceutical formulations comprising a compound of the invention or a pharmaceutically acceptable salt or derivative thereof together with one or more pharmaceutically acceptable carriers therefor. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof, and in such form can be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For oral administration, the pharmaceutical composition can be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water can be used as a suitable pharmaceutically acceptable carrier.

Compounds of the present invention or a solvate or physiologically functional derivative thereof can be used as active ingredients in pharmaceutical compositions, specifically as RUP25 receptor agonists. By the term “active ingredient” is defined in the context of a “pharmaceutical composition” and shall mean a component of a pharmaceutical composition that provides the primary pharmacological effect, as opposed to an “inactive ingredient” which would generally be recognized as providing no pharmaceutical benefit.

The dose when using the compounds of the present invention can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but not limited to, about 0.001 mg to about 5000 mg, about 0.001 to about 2500 mg, about 0.001 to about 1000 mg, 0.001 to about 500 mg, 0.001 mg to about 250 mg, about 0.001 mg to 100 mg, about 0.001 mg to about 50 mg, and about 0.001 mg to about 25 mg. Multiple doses can be administered during the day, especially when relatively large amounts are deemed to be needed, for example 2, 3 or 4, doses. Depending on the individual and as deemed appropriate from the patient's physician or care-giver it may be necessary to deviate upward or downward from the doses described herein.

The amount of active ingredient, or an active salt or derivative thereof, required for use in prophylaxis or treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system to another, for example, an animal model to a human. Typically, animal models include, but are not limited to, the rodents diabetes models as described in Example 15, infra; the mouse artherosclerosis model as described in Example 16, infra; or the in vivo animal arthosclerosis model as described in Example 17, infra. In some circumstances, these extrapolations may merely be based on the weight of the animal model in comparison to another, such as a mammal, preferably a human, however, more often, these extrapolations are not simply based on weight differences, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the Formula (I) and as part of a drug combination. The dosage regimen for treating a disease condition with the compounds and/or compositions of this invention is selected in accordance with a variety factors, such as, those cited above. Thus, the actual dosage regimen employed may vary widely and therefore may deviate from a preferred dosage regimen and one skilled in the art will recognize that dosage and dosage regimen outside these typical ranges can be tested and, where appropriate, can be used in the methods of this invention.

The desired dose may conveniently be presented in a single dose or as divided doses administered at appropriate intervals, for example, as two, three, four or more sub-doses per day. The sub-dose itself can be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it can be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration can be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions can be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and can be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention can be formulated as ointments, creams or lotions, or as a transdermal patch.

Ointments and creams may, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations can be provided in single or multi-dose form. In the latter case of a dropper or pipette, this can be achieved by the patient administering an appropriate, predetermined volume of the solution or suspension. In the case of a spray, this can be achieved for example by means of a metering atomizing spray pump.

Administration to the respiratory tract may also be achieved by means of an aerosol formulation in which the active ingredient is provided in a pressurized pack with a suitable propellant. If the compounds of the Formula (I) or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the Formula (I) as an aerosol can be prepared by processes well-known to the person skilled in the art. For their preparation, for example, solutions or dispersions of the compounds of the Formula (I) in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol may conveniently also contain a surfactant such as lecithin. The dose of drug can be controlled by provision of a metered valve.

In formulations intended for administration to the respiratory tract, including intranasal formulations, the compound will generally have a small particle size for example of the order of 10 microns or less. Such a particle size can be obtained by means known in the art, for example by micronization. When desired, formulations adapted to give sustained release of the active ingredient can be employed.

Alternatively the active ingredients can be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition can be presented in unit dose form for example in capsules or cartridges of, e.g., gelatin, or blister packs from which the powder can be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In general, the “pro-drug” approach is utilized to facilitate oral absorption. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Combination Therapy—Prophylaxis and Treatment:

While the compounds of the present invention can be administered as the sole active pharmaceutical agent (i.e., mono-therapy), they can also be used in combination with other pharmaceutical agents (i.e., combination-therapy), such as, for the treatment of the diseases/conditions/disorders described herein. Therefore, another aspect of the present invention includes methods of prophylaxis and/or treatment of metabolic related diseases comprising administering to an individual in need of such prophylaxis and/or treatment a therapeutically effective amount of a compound of the present invention in combination with one or more additional pharmaceutical agent as described herein.

Suitable pharmaceutical agents that can be used in combination with the compounds of the present invention include anti-obesity agents such as apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agensts, β₃ adrenergic receptor agonists, dopamine agonists (for example, bromocriptine), melanocyte-stimulating hormone receptor analogs, cannabinoid 1 receptor antagonists [for example, SR141716: N-(piperidin-1-yl)-5-(4-chlorophenyl)-1-(2,4-dichlorophenyl)-4-methyl-1H-pyrazole-3-carboxamide], melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion).

Other anti-obesity agents, including the agents set forth infra, are well known, or will be readily apparent in light of the instant disclosure, to one of ordinary skill in the art.

In some embodiments, the anti-obesity agents are selected from the group consisting of orlistat, sibutramine, bromocriptine, ephedrine, leptin, and pseudoephedrine. In a further embodiment, compounds of the present invention and combination therapies are administered in conjunction with exercise and/or a sensible diet.

It is understood that the scope of combination-therapy of the compounds of the present invention with other anti-obesity agents, anorectic agents, appetite suppressant and related agents is not limited to those listed above, but includes in principle any combination with any pharmaceutical agent or pharmaceutical composition useful for the treatment of overweight and obese individuals.

Other suitable pharmaceutical agents, in addition to anti-obesity agents, that can be used in combination with the compounds of the present invention include agents useful in the prophylaxis or treatment of concomitant disorders. Treatment of such disorders include the use of one or more pharmaceutical agents known in the art that belong to the classes of drugs referred to, but not limited to, the following: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin. In accordance to one aspect of the present invention, a compound of the present can be used in combination with a pharmaceutical agent or agents belonging to one or more of the classes of drugs cited herein.

It is understood that the scope of combination-therapy of the compounds of the present invention with other pharmaceutical agents is not limited to those listed herein, supra or infra, but includes in principle any combination with any pharmaceutical agent or pharmaceutical composition useful for the treatment of diseases, conditions or disorders that are linked to metabolic-related disorders.

Some embodiments of the present invention include methods of prophylaxis or treatment of a disease, disorder or condition as described herein comprising administering to an individual in need of such prophylaxis or treatment a therapeutically effect amount or dose of a compound of the present invention in combination with at least one pharmaceutical agent selected from the group consisting of: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin. In some embodiments, the pharmaceutical composition further comprises one or more agents selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

One aspect of the present invention encompasses pharmaceutical compositions comprising at least one compound according to Formula (I), as described herein. In some embodiments, the pharmaceutical composition further comprises one or more agents selected from the group consisting of, for example, α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

Suitable pharmaceutical agents that can be used in conjunction with compounds of the present invention include α-glucosidase inhibitors. α-Glucosidase inhibitors belong to the class of drugs which competitively inhibit digestive enzymes such as α-amylase, maltase, α-dextrinase, sucrase, etc. in the pancreas and or small intesting. The reversible inhibition by α-glucosidase inhibitors retard, diminish or otherwise reduce blood glucose levels by delaying the digestion of starch and sugars. Some representative examples of α-glucosidase inhibitors include acarbose, N-(1,3-dihydroxy-2-propyl)valiolamine (generic name; voglibose), miglitol, and α-glucosidase inhibitors known in the art.

Suitable pharmaceutical agents that can be used in conjunction with compounds of the present invention include sulfonylureas. The sulfonylureas (SU) are drugs which promote secretion of insulin from pancreatic β cells by transmitting signals of insulin secretion via SU receptors in the cell membranes. Examples of the sulfonylureas include glyburide, glipizide, glimepiride and other sulfonylureas known in the art.

Suitable pharmaceutical agents that can be used in conjunction with compounds of the present invention include the meglitinides. The meglitinides are benzoic acid derivatives represent a novel class of insulin secretagogues. These agents target postprandial hyperglycemia and show comparable efficacy to sulfonylureas in reducing HbA_{1c}. Examples of meglitinides include repaglinide, nateglinide and other meglitinides known in the art.

Suitable pharmaceutical agents that can be used in conjunction with compounds of the present invention include the biguanides. The biguanides represent a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin, metformin, buformin, and biguanides known in the art.

Suitable pharmaceutical agents that can be used in conjunction with compounds of the present invention include the α-glucosidase inhibitors. The α-glucosidase inhibitors competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Examples of α-glucosidase inhibitors include acarbose, N-1,3-dihydroxy-2-propyl)valiolamine (generic name; voglibose), miglitol, and α-glucosidase inhibitors known in the art.

Suitable pharmaceutical agents that can be used in conjunction with compounds of the present invention include the peroxisome proliferators-activated receptor-γ (i.e., PPAR-γ) agonists. The peroxisome proliferators-activated receptor-γ agonists represent a class of compounds that activates the nuclear receptor PPAR-γ and therefore regulate the transcription of insulin-responsive genes involved in the control of glucose production, transport and utilization. Agents in the class also facilitate the regulation of fatty acid metabolism. Examples of PPAR-γ agonists include rosiglitazone, pioglitazone, tesaglitazar, netoglitazone, GW409544, GW-501516 and PPAR-γ agonists known in the art.

Suitable pharmaceutical agents that can be used in conjunction with compounds of the present invention include the HMG-CoA reductase inhibitors. The HMG-CoA reductase inhibitors are agents also referred to as Statin compounds that belong to a class of drugs that lower blood cholesterol levels by inhibiting hydroxymethylglutalyl CoA (HMG-CoA) reductase. HMG-CoA reductase is the rate-limiting enzyme in cholesterol biosynthesis. The statins lower serum LDL concentrations by upregulating the activity of LDL receptors and are responsible for clearing LDL from the blood. Some representative examples the statin compounds include rosuvastatin, pravastatin and its sodium salt, simvastatin, lovastatin, atorvastatin, fluvastatin, cerivastatin, rosuvastatin, pitavastatin, BMS's “superstatin”, and HMG-CoA reductase inhibitors known in the art.

Suitable pharmaceutical agents that can be used in conjunction with compounds of the present invention include the angiotensin converting enzyme (ACE) inhibitors. The angiotensin converting enzyme inhibitors belong to the class of drugs that partially lower blood glucose levels as well as lowering blood pressure by inhibiting angiotensin converting enzymes. Examples of the angiotensin converting enzyme inhibitors include captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and angiotensin converting enzyme inhibitors known in the art.

Suitable pharmaceutical agents that can be used in conjunction with compounds of the present invention include the angiotensin II receptor antagonists. Angiotensin II receptor antagonists target the angiotensin II receptor subtype 1 (i.e., AT1) and demonstrate a beneficial effect on hypertension. Examples of angiotensin II receptor antagonists include losartan (and the potassium salt form), and angiotensin II receptor antagonists known in the art.

Other treatments for one or more of the diseases cited herein include the use of one or more pharmaceutical agents known in the art that belong to the classes of drugs referred to, but not limited to, the following: amylin agonists (for example, pramlintide), insulin secretagogues (for example, GLP-1 agonists; exendin-4; insulinotropin (NN2211); dipeptyl peptidase inhibitors (for example, NVP-DPP-728), acyl CoA cholesterol acetyltransferase inhibitors (for example, Ezetimibe, eflucimibe, and like compounds), cholesterol absorption inhibitors (for example, ezetimibe, pamaqueside and like compounds), cholesterol ester transfer protein inhibitors (for example, CP-529414, JTT-705, CETi-1, and like compounds), microsomal triglyceride transfer protein inhibitors (for example, implitapide, and like compounds), cholesterol modulators (for example, NO-1886, and like compounds), bile acid modulators (for example, GT103-279 and like compounds) and squalene synthase inhibitors. Squalene synthesis inhibitors belong to a class of drugs that lower blood cholesterol levels by inhibiting synthesis of squalene. Examples of the squalene synthesis inhibitors include (S)-α-[Bis[2,2-methyl-1-oxopropoxy)methoxy]phosphinyl]-3-phenoxybenzenebutanesulfonic acid, mono potassium salt (BMS-188494) and squalene synthesis inhibitors known in the art.

In accordance with the present invention, the combination can be used by mixing the respective active components either all together or independently with a pharmaceutically acceptable carrier, excipient, binder, diluent, etc., as described herein above, and administering the mixture or mixtures either orally or non-orally as a pharmaceutical composition. When a compound or a mixture of compounds of Formula (I) are administered as a combination therapy or prophylaxis with another active compound the therapeutic agents can be formulated as separate pharmaceutical compositions given at the same time or at different times, or the therapeutic agents can be given as a single composition.

In accordance with the present invention, the combination of a compound of the present invention and pharmaceutical agent can be prepared by mixing the respective active components either all together or independently with a pharmaceutically acceptable carrier, excipient, binder, diluent, etc., as described herein, and administering the mixture or mixtures either orally or non-orally as a pharmaceutical composition. When a compound or a mixture of compounds of Formula (I) are administered as a combination therapy or prophylaxis with another active compound the therapeutic agents can be formulated as a separate pharmaceutical compositions given at the same time or at different times, or the therapeutic agents can be given as a single composition.

Other Utilities

Another object of the present invention relates to radio-labeled compounds of Formula (I) that are useful not only in radio-imaging but also in assays, both in vitro and in vivo, for localizing and quantitating RUP25 in tissue samples, including human, and for identifying RUP25 ligands by inhibition binding of a radio-labeled compound. It is a further object of this invention to include novel RUP25 assays of which comprise such radio-labeled compounds.

The present invention embraces isotopically-labeled compounds of Formula (I) and any subgenera herein, such as but not limited to, Formulae (Ia) to (Iz); (IIa) to (IIy); (IIIa) to (IIIt); and (IVa) to (IVs). An “isotopically” or “radio-labeled” compounds are those which are identical to compounds disclosed herein, but for the fact that one or more atoms are replaced or substituted by an atom having an atomic mass or mass number different from the atomic mass or mass number typically found in nature (i.e., naturally occurring). Suitable radionuclides that can be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro RUP25 labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled “or “labeled compound” is a compound of Formula (I) that has incorporated at least one radionuclide; in some embodiments the radionuclide is selected from the group consisting of ³H, ¹⁴C, ¹²⁵I, ³⁵S and ⁸²Br.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence can be preferred in some circumstances. Isotopically labeled compounds of the present invention can generally be prepared by following procedures analogous to those disclosed in the Schemes supra and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the more scarce radio-isotope or nonradio-active isotope.

Synthetic methods for incorporating radio-isotopes into organic compounds are applicable to compounds of the invention and are well known in the art. These synthetic methods, for example, incorporating activity levels of tritium into target molecules, and are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na²⁵I. A represented procedure was reported by Zhu, D.-G. and co-workers in J. Org. Chem. 2002, 67, 943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

A radio-labeled RUP25 compound of Formula (I) can be used in a screening assay to identify/evaluate compounds. In general terms, a newly synthesized or identified compound (i.e., test compound) can be evaluated for its ability to reduce binding of the “radio-labeled compound of Formula ([)” to the RUP25 receptor. Accordingly, the ability of a test compound to compete with the “radio-labeled compound of Formula (I)” for the binding to the RUP25 receptor directly correlates to its binding affinity.

The labeled compounds of the present invention bind to the RUP25 receptor. In one embodiment, the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM, and in still yet another embodiment the labeled inhibitor has an IC₅₀ less than about 0.1 μM.

Other uses of the disclosed receptors and methods will become apparent to those in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### EXAMPLES

The following Examples are provided for illustrative purposes and not as a means of limitation. One of ordinary skill in the art would be able to design equivalent assays and methods based on the disclosure herein, all of which form part of the present invention.

### Example 1

Full Length Cloning

hRUP25

The disclosed human hRUP25 was identified based upon the use of the GenBank database information. While searching the database, a cDNA clone with Accession Number AC026331 was identified as a human genomic sequence from chromosome 12. The full length hRUP25 was cloned by PCR using primers:

| | |
|---|---|
| 5′-GCTGGAGCATTCACTAGGCGAG-3′; | (SEQ. ID. NO.: 1 |
| sense, 5′ of initiation codon), | |
| 5′-AGATCCTGGTTCTTGGTGACAATG-3′; | (SEQ. ID. NO.: 2 |
| antisense, 3′ of stop codon) | |

and human genomic DNA (Promega) as template. Advantage cDNA polymerase mix (Clontech) was used for the amplification with 5% DMSO by the following cycle with step 2 to 4 repeated 35 times: 94° C. for 1 minute; 94° C. for 15 seconds; 56° C. for 20 seconds 72° C. for 1 minute 30 seconds and 72° C. for 5 minutes.

A 1.2 kb PCR fragment was isolated from a 1% agarose gel and cloned into the pCRII-TOPO vector (Invitrogen) and completely sequenced using the ABI Big Dye Terminator Kit (P.E. Biosystems).

### Example 2

Receptor Expression

Although a variety of cells are available to the art for the expression of proteins, it is most preferred that mammalian cells be utilized. The primary reason for this is predicated upon practicalities, i.e., utilization of, e.g., yeast cells for the expression of a GPCR, while possible, introduces into the protocol a non-mammalian cell which may not (indeed, in the case of yeast, does not) include the receptor-coupling, genetic-mechanism and secretary pathways that have evolved for mammalian systems—thus, results obtained in non-mammalian cells, while of potential use, are not as preferred as that obtained from mammalian cells. Of the mammalian cells, COS-7, 293 and 293T cells are particularly preferred, although the specific mammalian cell utilized can be predicated upon the particular needs of the artisan.

a. Transient Transfection

On day one, 6×10⁶/10 cm dish of 293 cells well were plated out. On day two, two reaction tubes were prepared (the proportions to follow for each tube are per plate): tube A was prepared by mixing 4 μg DNA (e.g., pCMV vector; pCMV vector with receptor cDNA, etc.) in 0.5 ml serum free DMEM (Gibco BRL); tube B was prepared by mixing 24 μl lipofectamine (Gibco BRL) in 0.5 ml serum free DMEM. Tubes A and B were admixed by inversions (several times), followed by incubation at room temperature for 30-45 min. The admixture is referred to as the “transfection mixture”. Plated 293 cells were washed with 1×PBS, followed by addition of 5 ml serum free DMEM. 1 ml of the transfection mixture were added to the cells, followed by incubation for 4 hrs at 37° C./5% CO₂. The transfection mixture was removed by aspiration, followed by the addition of 10 ml of DMEM/10% Fetal Bovine Serum. Cells were incubated at 37° C./5% CO₂. After 48 hr incubation, cells were harvested and utilized for analysis.

b. Stable Cell Lines: Gs Fusion Protein

Approximately 12×10⁶ 293 cells are plated on a 15 cm tissue culture plate. Grown in DME High Glucose Medium containing ten percent fetal bovine serum and one percent sodium pyruvate, L-glutamine, and anti-biotics. Twenty-four hours following plating of 293 cells (or to ˜80% confluency), the cells are transfected using 12 μg of DNA. The 12 μg of DNA is combined with 60 μl of lipofectamine and 2 mL of DME High Glucose Medium without serum. The medium is aspirated from the plates and the cells are washed once with medium without serum. The DNA, lipofectamine, and medium mixture are added to the plate along with 10 mL of medium without serum. Following incubation at 37 degrees Celsius for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of 500 μg/mL. The transfected cells now undergo selection for positively transfected cells containing the G418 resistant gene. The medium is replaced every four to five days as selection occurs. During selection, cells are grown to create stable pools, or split for stable clonal selection.

### Example 3

Assays For Determination of Constitutive Activity of Non-Endogenous GPCRs

A variety of approaches are available for assessment of constitutive activity of the non-endogenous human GPCRs. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Constitutively activated receptors continue to exchange GDP for GTP. The non-hydrolyzable GTP analog, [³⁵S]GTPγS, can be utilized to demonstrate enhanced binding of [³⁵S]GTPγS to membranes expressing constitutively activated receptors. The advantage of using [³⁵S]GTγS binding to measure constitutive activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used in the direct identification method to screen candidate compounds to known, orphan and constitutively activated G protein-coupled receptors. The assay is generic and has application to drug discovery at all G protein-coupled receptors.

The [³⁵S]GTγS assay was incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred ) and 12.5 to 75 μg membrane protein (e.g, 293 cells expressing the Gs Fusion Protein; this amount can be adjusted for optimization) and 10 μM GDP (this amount can be changed for optimization) for 1 hour. Wheatgerm agglutinin beads (25 μl; Amersham) were then added and the mixture incubated for another 30 minutes at room temperature. The tubes were then centrifuged at 1500×g for 5 minutes at room temperature and then counted in a scintillation counter.

2. Adenylyl Cyclase

A Flash Plate™ Adenylyl Cyclase kit (New England Nuclear; Cat. No. SP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells were harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells were pipetted off the plate and the cell suspension was collected into a 50 ml conical centrifuge tube. Cells were then centrifuged at room temperature at 1,100 rpm for 5 min. The cell pellet was carefully re-suspended into an appropriate volume of PBS (about 3 ml/plate). The cells were then counted using a hemocytometer and additional PBS was added to give the appropriate number of cells (with a final volume of about 50 μl/well).

cAMP standards and Detection Buffer [comprising 1 μCi of tracer ¹²⁵I-cAMP (50 μl) to 11 ml Detection Buffer) was prepared and maintained in accordance with the manufacturer's instructions. Assay Buffer was prepared fresh for screening and contained 50 μl of Stimulation Buffer, 3 ul of test compound (12 μM final assay concentration) and 50 μl cells, Assay Buffer was stored on ice until utilized. The assay was initiated by addition of 50 μl of cAMP standards to appropriate wells followed by addition of 50 ul of PBSA to wells H-11 and H12. 50 μl of Stimulation Buffer was added to all wells. DMSO (or selected candidate compounds) was added to appropriate wells using a pin tool capable of dispensing 3 μl of compound solution, with a final assay concentration of 12 μM test compound and 100 μl total assay volume. The cells were then added to the wells and incubated for 60 min at room temperature. 100 μl of Detection Mix containing tracer cAMP was then added to the wells. Plates were then incubated additional 2 hours followed by counting in a Wallac MicroBeta scintillation counter. Values of cAMP/well were then extrapolated from a standard cAMP curve which was contained within each assay plate.

3. Cell-Based cAMP for Gi Coupled Target GPCRs

TSHR is a Gs coupled GPCR that causes the accumulation of cAMP upon activation. TSHR will be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of constitutive activation of a Gi coupled receptor can be accomplished by co-transfecting, most preferably, non-endogenous, constitutively activated TSHR (TSHR-A6231) (or an endogenous, constitutively active Gs coupled receptor) as a “signal enhancer” with a Gi linked target GPCR to establish a baseline level of cAMP. Upon creating a non-endogenous version of the Gi coupled receptor, this non-endogenous version of the target GPCR is then co-transfected with the signal enhancer, and it is this material that can be used for screening. We will utilize such approach to effectively generate a signal when a cAMP assay is used; this approach is preferably used in the direct identification of candidate compounds against Gi coupled receptors. It is noted that for a Gi coupled GPCR, when this approach is used, an inverse agonist of the target GPCR will increase the cAMP signal and an agonist will decrease the cAMP signal.

On day one, 2×10⁴ 293 cells/well will be plated out. On day two, two reaction tubes will be prepared (the proportions to follow for each tube are per plate): tube A will be prepared by mixing 2 μg DNA of each receptor transfected into the mammalian cells, for a total of 4 μg DNA (e.g., pCMV vector; pCMV vector with mutated THSR (TSHR-A623I); TSHR-A623I and GPCR, etc.) in 1.2 ml serum free DMEM (Irvine Scientific, Irvine, Calif.); tube B will be prepared by mixing 120 μl lipofectamine (Gibco BRL) in 1.2 ml serum free DMEM. Tubes A and B will then be admixed by inversions (several times), followed by incubation at room temperature for 30-45min. The admixture is referred to as the “transfection mixture”. Plated 293 cells will be washed with 1×PBS, followed by addition of 10 ml serum free DMEM. 2.4 ml of the transfection mixture will then be added to the cells, followed by incubation for 4 hrs at 37° C./5% CO₂. The transfection mixture will then be removed by aspiration, followed by the addition of 25 ml of DMEM/10% Fetal Bovine Serum. Cells will then be incubated at 37° C./5% CO₂. After 24 hr incubation, cells will then be harvested and utilized for analysis.

A Flash Plate™ Adenylyl Cyclase kit (New England Nuclear; Cat. No. SMP004A) is designed for cell-based assays, however, can be modified for use with crude plasma membranes depending on the need of the skilled artisan. The Flash Plate wells will contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells will be harvested approximately twenty four hours after transient transfection. Media will be carefully aspirated off and discarded. 10 ml of PBS will be gently added to each dish of cells followed by careful aspiration. 1 ml of Sigma cell dissociation buffer and 3 ml of PBS will be added to each plate. Cells will be pipetted off the plate and the cell suspension will be collected into a 50 ml conical centrifuge tube. Cells will then be centrifuged at room temperature at 1,100 rpm for 5 min. The cell pellet will be carefully re-suspended into an appropriate volume of PBS (about 3 ml/plate). The cells will then be counted using a hemocytometer and additional PBS is added to give the appropriate number of cells (with a final volume of about 50 μl/well).

cAMP standards and Detection Buffer [comprising 1 μCi of tracer ¹²⁵I-cAMP (50 μl) to 11 ml Detection Buffer] will be prepared and maintained in accordance with the manufacturer's instructions. Assay Buffer should be prepared fresh for screening and contained 50 μl of Stimulation Buffer, 3 μl of test compound (12 μM final assay concentration) and 50 μl cells, Assay Buffer can be stored on ice until utilized. The assay can be initiated by addition of 50 μl of cAMP standards to appropriate wells followed by addition of 50 μl of PBSA to wells H-11 and H12. Fifty μl of Stimulation Buffer will be added to all wells. Selected compounds (e.g., TSH) will be added to appropriate wells using a pin tool capable of dispensing 3 μl of compound solution, with a final assay concentration of 12 μM test compound and 100 μl total assay volume. The cells will then be added to the wells and incubated for 60 min at room temperature. 100 μl of Detection Mix containing tracer cAMP will then be added to the wells. Plates were then incubated additional 2 hours followed by counting in a Wallac MicroBeta scintillation counter. Values of cAMP/well will then be extrapolated from a standard cAMP curve which is contained within each assay plate.

### Example 4

Tissue Distribution of the Disclosed Human GPCRs

A. RT-PCR

RT-PCR was applied to confirm the expression and to determine the tissue distribution of several novel human GPCRs. Oligonucleotides utilized were GPCR-specific and the human multiple tissue cDNA panels (MTC, Clontech) as templates. Taq DNA polymerase (Stratagene) were utilized for the amplification in a 40 μl reaction according to the manufacturer's instructions. 20 μl of the reaction will be loaded on a 1.5% agarose gel to analyze the RT-PCR products. Table B below lists the receptors, the cycle conditions and the primers utilized.

TABLE B

| | Cycle | | | | |
|---|---|---|---|---|---|
| | Condi- | | | | |
| | tions | | | | |
| | Min (′), | | | | |
| | Sec (″) | | 3′ | | |
| Recep- | Cycles | | Primer | | |
| tor | 2-4 | 5′ Primer | (SEQ. | DNA | |
| Iden- | repeated | (SEQ. ID. | ID. | Frag- | Tissue |
| tifier | 30 times | NO.) | NO.) | ment | Expression |
| hRUP25 | 96° | CTGATGGA | GCTGAAGC | 297 bp | Adipocyte, |
| | for 2′ | | | | |
| | 96° | CAACTATGT | TGCTGCAC | | spleen, |
| | for 30″ | | | | |
| | 55° C. | GAGGCGTT | AAATTTGC | | leukocyte, |
| | for 1′ | | | | |
| | 72° | GG (3) | ACC (4) | | kidney, |
| | for 2′ | | | | |
| | 72° | | | | lung, |
| | for 10′ | | | | testis |

Diseases and disorders related to receptors located in these tissues or regions include, but are not limited to, cardiac disorders and diseases (e.g. thrombosis, myocardial infarction; atherosclerosis; cardiomyopathies); kidney disease/disorders (e.g., renal failure; renal tubular acidosis; renal glycosuria; nephrogenic type 2 diabetes insipidus; cystinuria; polycystic kidney disease); eosinophilia; leukocytosis; leukopenia; ovarian cancer; sexual dysfunction; polycystic ovarian syndrome; pancreatitis and pancreatic cancer; irritable bowel syndrome; colon cancer, Crohn's disease; ulcerative colitis; diverticulitis; Chronic Obstructive Pulmonary Disease (COPD); Cystic Fibrosis; pneumonia; pulmonary hypertension; tuberculosis and lung cancer; Parkinson's disease; movement disorders and ataxias; learning and memory disorders; eating disorders (e.g., anorexia; bulimia, etc.); obesity; cancers; thymoma; myasthenia gravis; circulatory disorders; prostate cancer; prostatitis; kidney disease/disorders(e.g., renal failure; renal tubular acidosis; renal glycosuria; nephrogenic type 2 diabetes insipidus; cystinuria; polycystic kidney disease); sensorimotor processing and arousal disorders; obsessive-compulsive disorders; testicular cancer; priapism; prostatitis; hernia; endocrine disorders; sexual dysfunction; allergies; depression; psychotic disorders; migraine; reflux; schizophrenia; ulcers; bronchospasm; epilepsy; prostatic hypertrophy; anxiety; rhinitis; angina; and glaucoma. Accordingly, the methods of the present invention may also be useful in the diagnosis and/or treatment of these and other diseases and disorders.

B. Affymetrix GeneChip® Technology

Amino acid sequences were submitted to Affymetrix for the designing and manufacturing of microarray containing oligonucleotides to monitor the expression levels of G protein-coupled receptors (GPCRs) using their GeneChip® Technology. Also present on the microaccray were probes for characterized human brain tissues from Harvard Brain Band or obtained from commercially available sources. RNA samples were amplified, labeled, hybridized to the microarray, and data analyzed according to manufacturer's instructions.

Adipose tissues were monitored for the level of gene expression of each of the GPCRs represented on the microarray. GPCRs were determined to be expressed if the expression index was greater than 100 (based upon and according to manufacturer's instructions). The data was analyzed and had indicated that classification of GPCRs with an expression index greater than 100 was reasonable because a number of known GPCRs had previously been reported to be expressed in neuronal tissues with an expression index greater than 100.

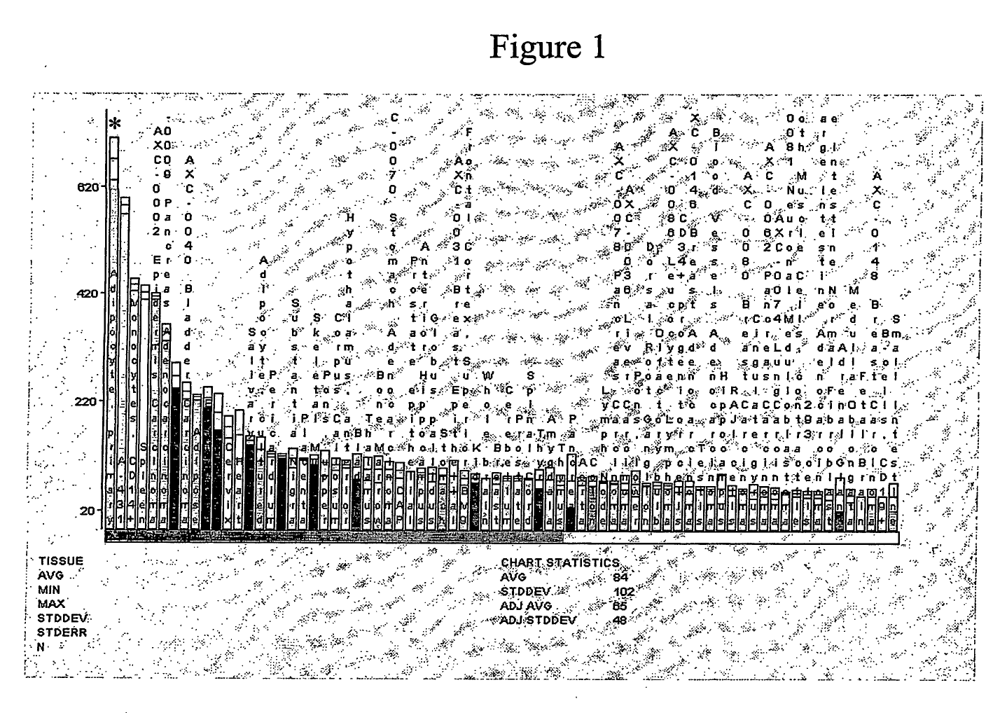

Using the GeneChip, we discovered hRUP25 to have high levels of expression in adipocytes suggesting that, for example, that hRUP25 may play a role in lipolysis (see, Goodman & Gilman's, The Pharmacological Basis of Therapeutics 9^{th} Edition, page 235 (1996). See FIG. 1. FIG. 1 is a plot representing the expression level of hRUP25 in various tissues. Based upon this data, hRUP25 is highly expressed by primary adipocytes.

This patent document discloses the identification of nicotinic acid as a ligand and agonist of human, mouse and rat RUP25. See, Examples infra.

### Example 5

Protocol: Direct Identification of Inverse Agonists and Agonists

A. [³⁵S]GTPγS Assay

Although we have utilized endogenous, constitutively active GPCRs for the direct identification of candidate compounds as, e.g., inverse agonists, for reasons that are not altogether understood, intra-assay variation can become exacerbated. In some embodiments, a GPCR Fusion Protein, as disclosed above, is also utilized with a non-endogenous, constitutively activated GPCR. When such a protein is used, intra-assay variation appears to be substantially stabilized, whereby an effective signal-to-noise ratio is obtained. This has the beneficial result of allowing for a more robust identification of candidate compounds. Thus, in some embodiments it is preferred that for direct identification, a GPCR Fusion Protein be used and that when utilized, the following assay protocols be utilized.

1. Membrane Preparation

In some embodiments membranes comprising the constitutively active orphan GPCR/Fusion Protein of interest and for use in the direct identification of candidate compounds as inverse agonists or agonists are preferably prepared as follows:

a. Materials

“Membrane Scrape Buffer” is comprised of 20 mM HEPES and 10 mM EDTA, pH 7.4;

“Membrane Wash Buffer” is comprised of 20 mM HEPES and 0.1 mM EDTA, pH 7.4;

“Binding Buffer” is comprised of 20 mM HEPES, 100 mM NaCl, and 10 mM MgCl₂, pH 7.4

b. Procedure

All materials will be kept on ice throughout the procedure. Firstly, the media will be aspirated from a confluent monolayer of cells, followed by rinse with 10 ml cold PBS, followed by aspiration. Thereafter, 5 ml of Membrane Scrape Buffer will be added to scrape cells; this will be followed by transfer of cellular extract into 50 ml centrifuge tubes (centrifuged at 20,000 rpm for 17 minutes at 4° C.). Thereafter, the supernatant will be aspirated and the pellet will be resuspended in 30 ml Membrane Wash Buffer followed by centrifuge at 20,000 rpm for 17 minutes at 4° C. The supernatant will then be aspirated and the pellet resuspended in Binding Buffer. This will then be homogenized using a Brinkman Polytron™ homogenizer (15-20 second bursts until the all material is in suspension). This is referred to herein as “Membrane Protein”.

2. Bradford Protein Assay

Following the homogenization, protein concentration of the membranes will be determined using the Bradford Protein Assay (protein can be diluted to about 1.5 mg/ml, aliquoted and frozen (−80° C.) for later use; when frozen, protocol for use will be as follows: on the day of the assay, frozen Membrane Protein is thawed at room temperature, followed by vortex and then homogenized with a Polytron at about 12×1,000 rpm for about 5-10 seconds; it was noted that for multiple preparations, the homogenizor should be thoroughly cleaned between homogenization of different preparations).

a. Materials

Binding Buffer (as per above); Bradford Dye Reagent; Bradford Protein Standard will be utilized, following manufacturer instructions (Biorad, cat. no. 500-0006).

b. Procedure

Duplicate tubes will be prepared, one including the membrane, and one as a control “blank”. Each contained 800 μl Binding Buffer. Thereafter, 10 μl of Bradford Protein Standard (1 mg/ml) will be added to each tube, and 10 μl of membrane Protein will then be added to just one tube (not the blank). Thereafter, 200 μl of Bradford Dye Reagent will be added to each tube, followed by vortex of each. After five (5) minutes, the tubes will be re-vortexed and the material therein will be transferred to cuvettes. The cuvettes will then be read using a CECIL 3041 spectrophotometer, at wavelength 595.

3. Direct Identification Assay

a. Materials

GDP Buffer consisted of 37.5 ml Binding Buffer and 2mg GDP (Sigma, cat. no. G-7127), followed by a series of dilutions in Binding Buffer to obtain 0.2 μM GDP (final concentration of GDP in each well was 0.1 μM GDP); each well comprising a candidate compound, has a final volume of 200 μl consisting of 100 μl GDP Buffer (final concentration, 0.1 μM GDP), 50 μl Membrane Protein in Binding Buffer, and 50 μl [³⁵S]GTPγS (0.6 nM) in Binding Buffer (2.5 μl [³⁵S]GTPγS per 10 ml Binding Buffer).

b. Procedure

Candidate compounds will be preferably screened using a 96-well plate format (these can be frozen at −80° C.). Membrane Protein (or membranes with expression vector excluding the GPCR Fusion Protein, as control), will be homogenized briefly until in suspension. Protein concentration will then be determined using the Bradford Protein Assay set forth above. Membrane Protein (and control) will then be diluted to 0.25 mg/ml in Binding Buffer (final assay concentration, 12.5 μg/well). Thereafter, 100 μl GDP Buffer was added to each well of a Wallac Scintistrip™ (Wallac). A 5 ul pin-tool will then be used to transfer 5 μl of a candidate compound into such well (i.e., 5 μl in total assay volume of 200 μl is a 1:40 ratio such that the final screening concentration of the candidate compound is 10 μM). Again, to avoid contamination, after each transfer step the pin tool should be rinsed in three reservoirs comprising water (1×), ethanol (1×) and water (2×)—excess liquid should be shaken from the tool after each rinse and dried with paper and kimwipes. Thereafter, 50 μl of Membrane Protein will be added to each well (a control well comprising membranes without the GPCR Fusion Protein was also utilized), and pre-incubated for 5-10 minutes at room temperature. Thereafter, 50 μl of [³⁵S]GTPγS (0.6 nM) in Binding Buffer will be added to each well, followed by incubation on a shaker for 60 minutes at room temperature (again, in this example, plates were covered with foil). The assay will then be stopped by spinning of the plates at 4000 RPM for 15 minutes at 22° C. The plates will then be aspirated with an 8 channel manifold and sealed with plate covers. The plates will then be read on a Wallac 1450 using setting “Prot. #37” (as per manufacturer instructions).

B. Cyclic AMP Assay

Another assay approach to directly identified candidate compound was accomplished by utilizing a cyclase-based assay. In addition to direct identification, this assay approach can be utilized as an independent approach to provide confirmation of the results from the [³⁵S]GTPγS approach as set forth above.

A modified Flash Plate™ Adenylyl Cyclase kit (New England Nuclear; Cat. No. SMP004A) was preferably utilized for direct identification-of candidate compounds as inverse agonists and agonists to constitutively activated orphan GPCRs in accordance with the following protocol.

Transfected cells were harvested approximately three days after transfection. Membranes were prepared by homogenization of suspended cells in buffer containing 20 mM HEPES, pH 7.4 and 10 mM MgCl₂. Homogenization was performed on ice using a Brinkman Polytron™ for approximately 10 seconds. The resulting homogenate is centrifuged at 49,000×g for 15 minutes at 4° C. The resulting pellet was then resuspended in buffer containing 20 mM HEPES, pH 7.4 and 0.1 mM EDTA, homogenized for 10 seconds, followed by centrifugation at 49,000×g for 15 minutes at 4° C. The resulting pellet was then stored at −80° C. until utilized. On the day of direct identification screening, the membrane pellet was slowly thawed at room temperature, resuspended in buffer containing 20 mM HEPES, pH 7.4 and 10 mM MgCl₂, to yield a final protein concentration of 0.60 mg/ml (the resuspended membranes are placed on ice until use).

cAMP standards and Detection Buffer [comprising 2 μCi of tracer ¹²⁵I-cAMP (100 μl) to 11 ml Detection Buffer] were prepared and maintained in accordance with the manufacturer's instructions. Assay Buffer was prepared fresh for screening and contained 20 mM HEPES, pH 7.4, 10 mM MgCl₂, 20 mM phospocreatine (Sigma), 0.1 units/ml creatine phosphokinase (Sigma), 50 μM GTP (Sigma), and 0.2 mM ATP (Sigma); Assay Buffer was then stored on ice until utilized.

Candidate compounds identified as per above (if frozen, thawed at room temperature) were added, preferably, to 96-well plate wells (3 μl/well; 12 μM final assay concentration), together with 40 μl Membrane Protein (30 μg/well) and 50 μl of Assay Buffer. This admixture was then incubated for 30 minutes at room temperature, with gentle shaking.

Following the incubation, 100 μl of Detection Buffer was added to each well, followed by incubation for 2-24 hours. Plates were then counted in a Wallac MicroBeta™ plate reader using “Prot #31” (as per manufacturer instructions).

### Example 6

Melanophore Technology

Melanophores are skin cells found in lower vertebrates. They contain pigmented organelles termed melanosomes. Melanophores are able to redistribute these melanosomes along a microtubule network upon G-protein coupled receptor (GPCR) activation. The result of this pigment movement is an apparent lightening or darkening of the cells. In melanophores, the decreased levels of intracellular cAMP that result from activation of a Gi-coupled receptor cause melanosomes to migrate to the center of the cell, resulting in a dramatic lightening in color. If cAMP levels are then raised, following activation of a Gs-coupled receptor, the melanosomes are re-dispersed and the cells appear dark again. The increased levels of diacylglycerol that result from activation of Gq-coupled receptors can also induce this redispersion. In addition, the technology is also suited to the study of certain receptor tyrosine kinases. The response of the melanophores takes place within minutes of receptor activation and results in a simple, robust color change. The response can be easily detected using a conventional absorbance microplate reader or a modest video imaging system. Unlike other skin cells, the melanophores derive from the neural crest and appear to express a full complement of signaling proteins. In particular, the cells express an extremely wide range of G-proteins and so are able to functionally express almost all GPCRs.

Melanophores can be utilized to identify compounds, including natural ligands, against GPCRs. This method can be conducted by introducing test cells of a pigment cell line capable of dispersing or aggregating their pigment in response to a specific stimulus and expressing an exogenous clone coding for the GCPR. A stimulant, e.g., melatonin, sets an initial state of pigment disposition wherein the pigment is aggregated within the test cells if activation of the GPCR induces pigment dispersion. However, stimulating the cell with a stimulant to set an initial state of pigment disposition wherein the pigment is dispersed if activation of the GPCR induces pigment aggregation. The test cells are then contacted with chemical compounds, and it is determined whether the pigment disposition in the cells changed from the initial state of pigment disposition. Dispersion of pigments cells due to the candidate compound, including but not limited to a ligand, coupling to the GPCR will appear dark on a petri dish, while aggregation of pigments cells will appear light.

Materials and methods will be followed according to the disclosure of U.S. Pat. No. 5,462,856 and U.S. Pat. No. 6,051,386. These patent disclosures are hereby incorporated by reference in their entirety.

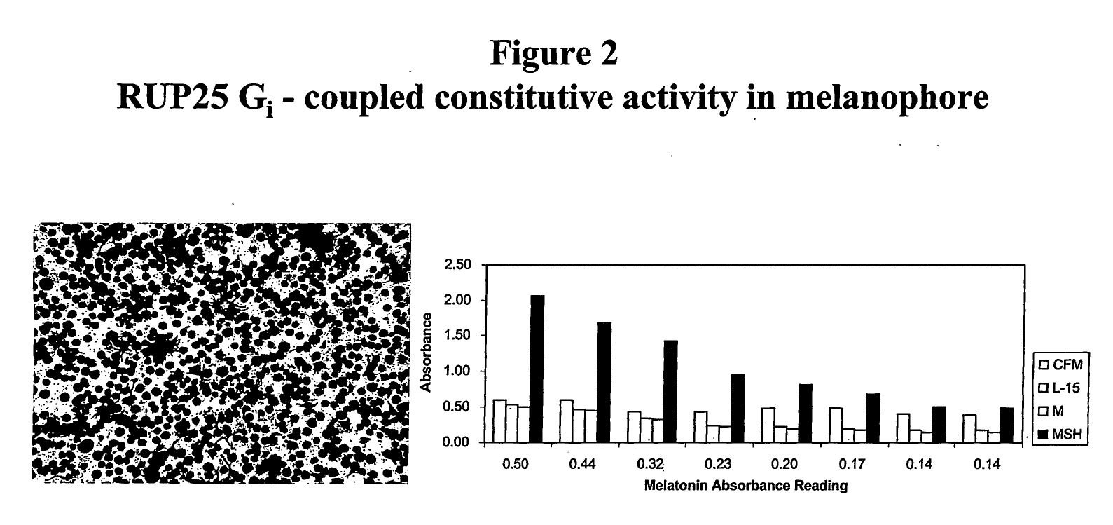

Melanophores were transfected by electroporation with plasmids coding for the GPCRs, for example hRUP25. Pre-screening of the GPCRs in melanophores was performed in the absence of nicotinic acid following the protocol below to determine the G protein coupling. This pre-screen evidenced that hRUP25 (FIG. 2) is strongly Gi-coupled.

The cells were plated in 96-well plates (one receptor per plate). 48 hours post-transfection, half of the cells on each plate were treated with 10 nM melatonin. Melatonin activates an endogenous Gi-coupled receptor in the melanophores and causes them to aggregate their pigment. The remaining half of the cells were transferred to serum-free medium 0.7×L-15 (Gibco). After one hour, the cells in serum-free media remained in a pigment-dispersed state while the melatonin-treated cells were in a pigment-aggregated state. At this point, the cells were treated with a dose response of nicotinic acid (Sigma). If the plated GPCRs bound to nicotinic acid, the melanophores would be expected to undergo a color change in response to the compound. If the receptor were either a Gs or Gq coupled receptor, then the melatonin-aggregated melanophores would undergo pigment dispersion. In contrast, if the receptor was a Gi-coupled receptor, then the pigment-dispersed cells would be expected to undergo a dose-dependent pigment aggregation.

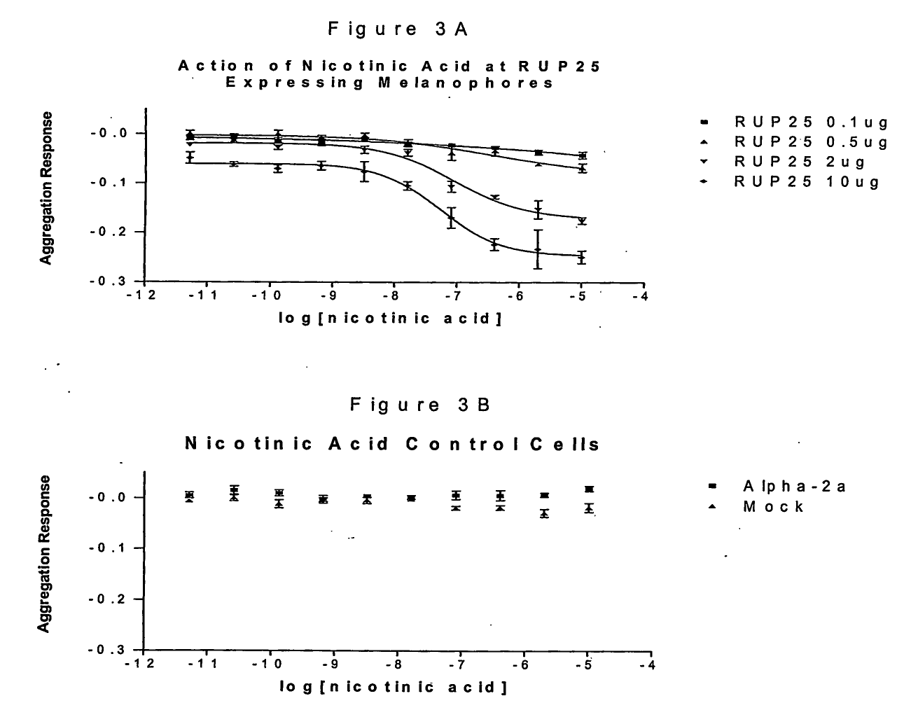

Melanophores transfected with hRUP25 were treated with nicotinic acid. Upon this treatment, the cells underwent pigment aggregation in a dose-dependent manner. hRUP25-expressing cells that were pre-aggregated with melatonin did not disperse upon nicotinic acid treatment, which is consistent with the receptor being a Gi-coupled receptor. See, FIG. 3 and infra.

To confirm and extend these results, melanophores were transfected with a range of hRUP25 DNA from 0 to 10 g. As controls, melanophores were also transfected with 10 μg of α_{2A} Adrenergic receptor (a known Gi-coupled receptor) and salmon sperm DNA (Gibco), as a mock transfection. On day 3, the cells were again incubated for 1 hour in serum-free L-15 medium (Gibco) and remained in a pigment-dispersed state. The cells were then treated with a dose response of nicotinic acid. See, FIG. 3A. FIG. 3A depicts the aggregation response of nicotinic acid at melanophores transfected with various ranges of hRUP25. At 10 μg of hRUP25, the EC₅₀ for nicotinic acid is about 54 nM. Stated differently, at very low concentrations, nicotinic acid evidences binding to hRUP25.

Reference is now made to FIG. 3B. In FIG. 3B, both the mock transfected and α_{2A} transfected cells did not respond to nicotinic acid. This data evidences that nicotinic acid binds specifically to the Gi-coupled receptor hRUP25.

The data show that the greater the amount of hRUP25 plasmid DNA transfected, the greater the magnitude of the observed aggregation response. Collectively these data indicate that hRUP25: 1) is a Gi-coupled receptor that 2) binds to nicotinic acid.

As set forth herein, nicotinic acid is a ligand for, and agonist of, human, mouse and rat RUP25. It is further shown that human, mouse and rat RUP25 are Gi-coupled. Additionally, human, mouse, and rat RUP25 can be used in methods described herein to identify antagonists, agonists, inverse agonists, partial agonists, allosteric enhancers, and negative allosteric modulators. As discussed supra, methods of modifying nicotinic acid receptor activity in adipocytes using a modulator of the receptor are set forth. Preferably, the modulator is an agonist.

### Example 7

Nicotinic Acid Induced-Inositol Phosphates Accumulation in 293 Cells Co-Expressing HRUP and GQΔGI

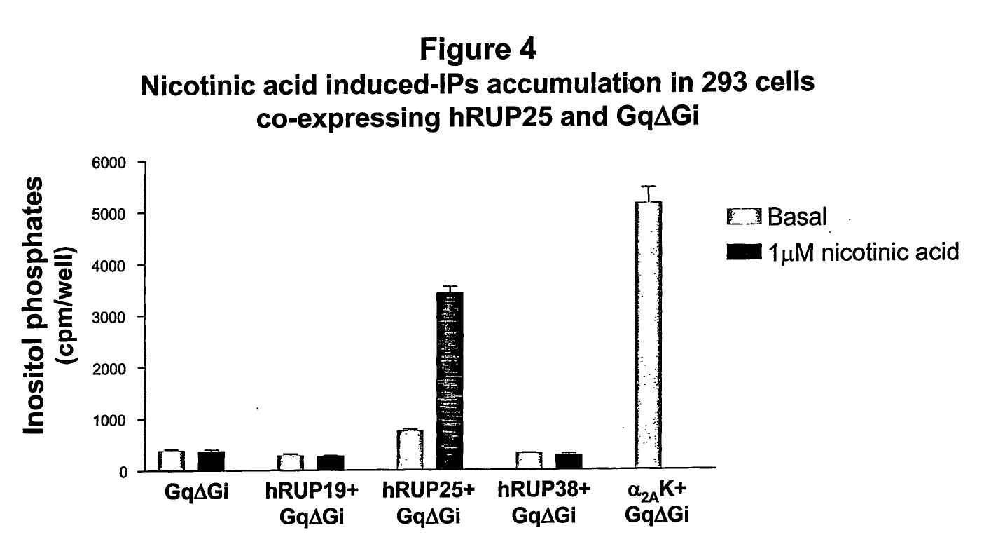

FIG. 4 illustrates the nicotinic acid induced-inositol phosphates (IPs) accumulation in HEK293 cells co-expressing hRUP25 and the chimeric Gαq-subunit in which the last five amino acids have been replaced with the corresponding amino acids of Gαi (GqΔGi). This construct has been shown to convert the signaling of a Gi-coupled receptor to the Gq pathway (i.e. accumulation of inositol phosphates) in response to receptor activation. Cells transfected with GqΔGi plus either empty plasmid or the constitutively activated α_{2A}AR (α_{2A}K) served as controls for the IP assay which are non-responsive to nicotinic acid.

### Example 8

Nicotinic Acid and Nicotine Induced-Inhibition of Forskolin Stimulated CAMP Accumulation in HRUP25-CHO Cell Stable Line #46

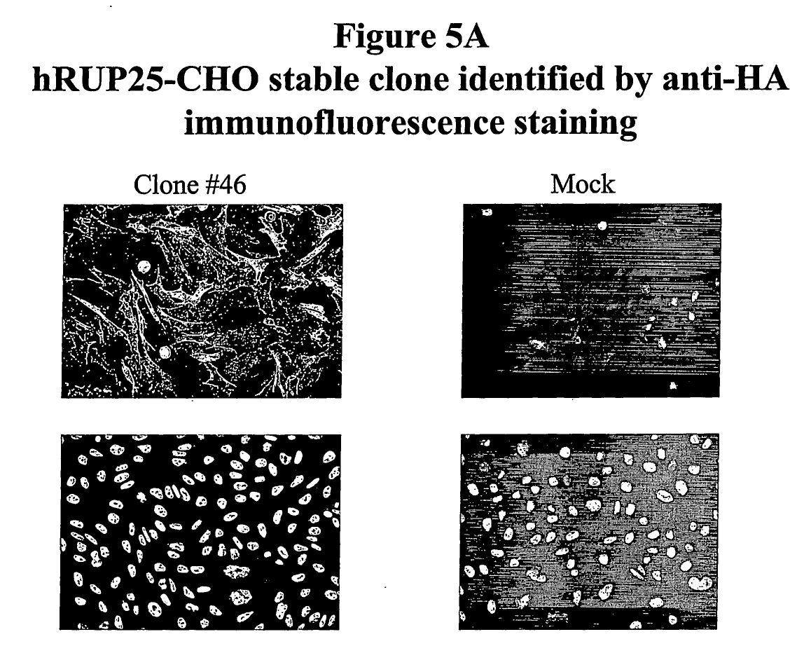

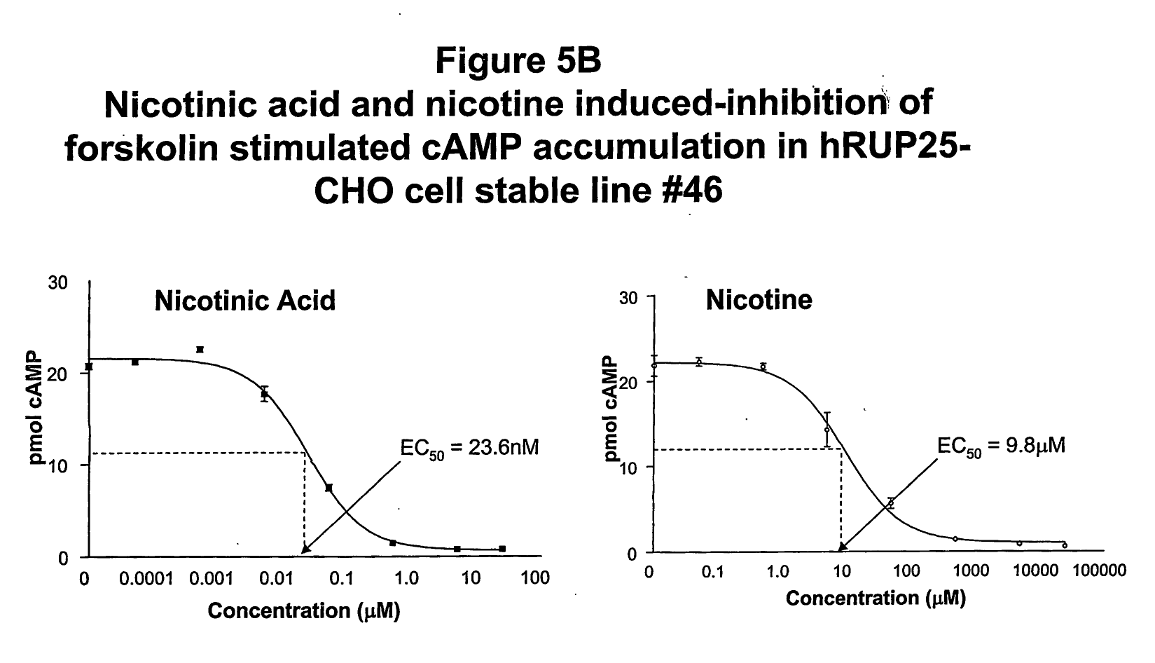

FIG. 5A is a set of immunofluorescent photomicrographs illustrating the expression of hemaglutinin(HA)-tagged hRUP25 in a stably transfected line of CHO cells (top; clone #46). No significant labeling is detected in mock stably-transfected CHO cells (Mock). The lower panels identify he nuclear (DAPI) staining of cells in the same field.

FIG. 5B illustrates nicotinic acid and nicotine induced-inhibition of forskolin stimulated cAMP accumulation in hRUP25-CHO cell stable line #46 (described in preceding paragraph). The EC₅₀ for nicotinic acid is 23.6 nM and that for nicotine is 9.8 μM.

### Example 9

hRUP25 AND mRUP25 Inhibit TSHR Induced-CAMP Accumulation Following Activation by Nicotinic Acid

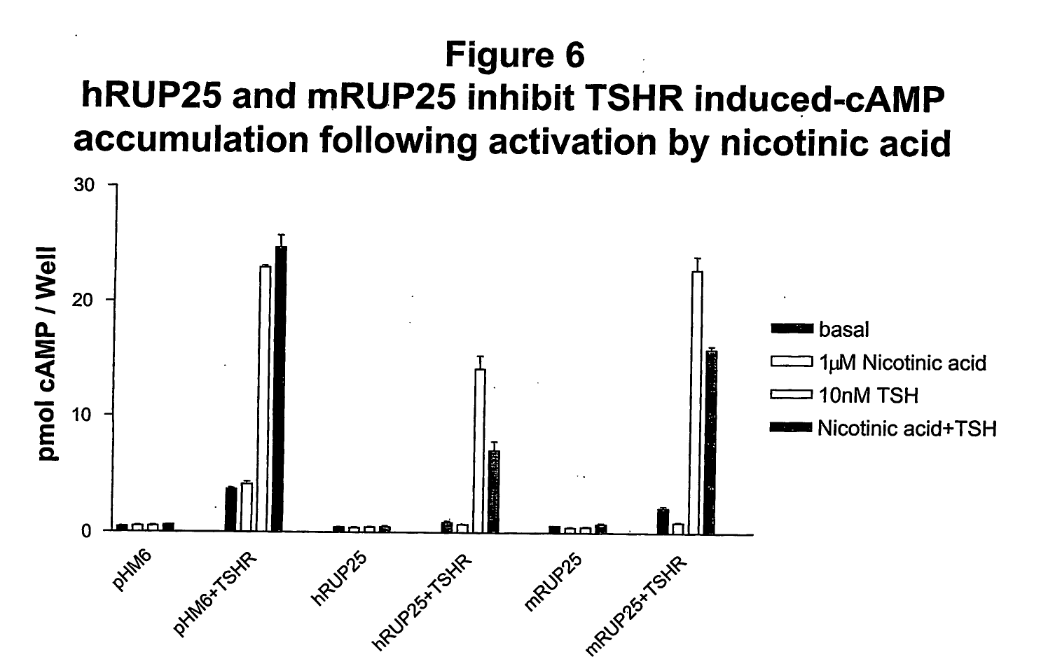

FIG. 6 indicates that, in response to nicotinic acid, both hRUP25 and the mouse ortholog mRUP25 can inhibit TSHR stimulated cAMP production (in the presence and absence of TSH).

### Example 10

hRUP25 and mRUP25 Bind to Nicotinic Acid Specifically and with High Affinity

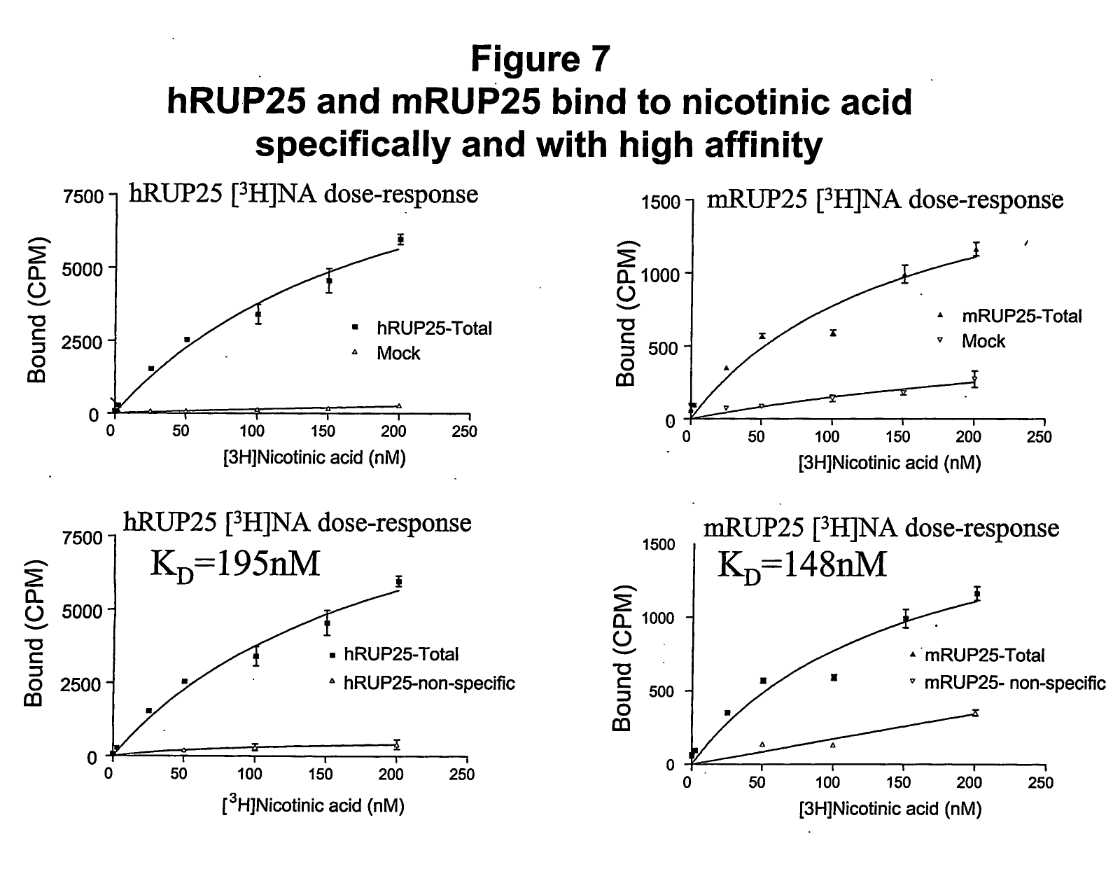

FIG. 7 shows the saturation binding curves of [³H]nicotinic acid ([³H]NA) to membranes prepared from HEK293 cells transiently expressing either hRUP25 or mRUP25. Note the significant binding of [³H]NA relative to either that found in membranes derived from mock transfected cells or in the presence of an excess of non-labeled nicotinic acid (200 μM).

Radioligand binding was done as follows. Media was removed from cells grown in culture [either stably or transiently transfected with negative control (empty plasmid) or with the individual receptors hRUP25, mRUP25, rRUP25 and cells were scraped and homogenized in buffer containing 15 mM HEPES, 5 mM EDTA, 5 mM EGTA, plus protease inhibitors (1eupeptin, PMSF and pepstatin). Membranes were harvested following centrifugation at 30,000×g, 4° C. for 30 min. Membranes were then resuspended and re-homogenized in CHAPS binding buffer (50 mM Tris-HCl and 0.02% CHAPS, pH 7.4). Aliquots were taken for protein analysis via the Bradford protein assay and normalized such that each binding reaction contained the same amount of membrane protein (25-50 μg). 50 nM [³H]nicotinic acid was added to each sample and either buffer (for total samples) or a desired amount of non-labeled compound (at the same volumes and in the same diluent) was added and the reactions were left at room temperature gently shaking for 1 hr. Free ligand was separated from bound ligand via rapid filtration onto a filter. Appropriate scintilant was added to each sample and counted in an appropriate scintillation counter. Data was analyzed using Excel and PrismGraph. In some cases radioligand binding was performed using a scintillation proximity assay (SPA) in which case the samples did not require filtration or the addition of scintilant.

### Example 11

The Rank Order of Potency of Compounds on hRUP25 Closely Matches That of the Pharmacologically Defined Nicotinic Acid Receptor

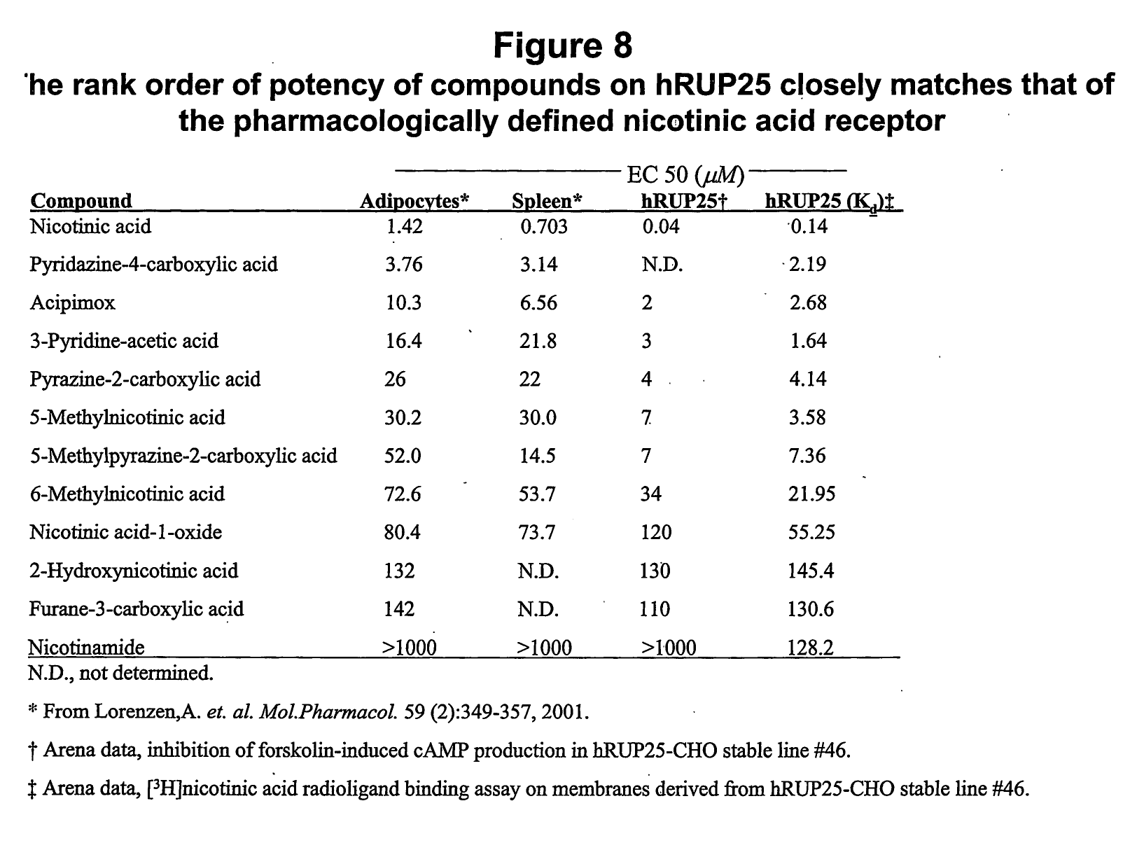

FIG. 8 is a table comparing the rank order of potency of various compounds on hRUP25 and the pharmacologically defined nicotinic acid receptor. The potencies at hRUP25 derived both by a functional analysis measuring the inhibition of forskolin induced cAMP production and competitive radioligand binding assays, closely match the order of potencies of the pharmacologically defined nicotinic acid receptor.

### Example 12

Nicotinic Acid and Related Compounds Inhibit Isoproterenol Induced Lipolysis in Rat Epidimal Fat Derived Adipocytes

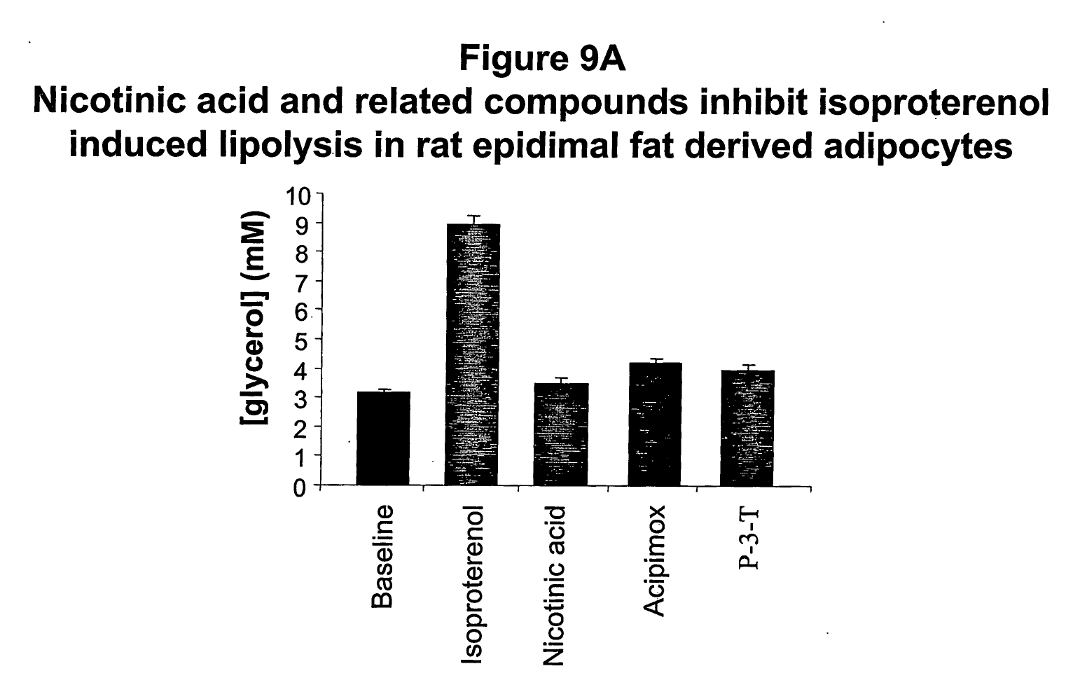

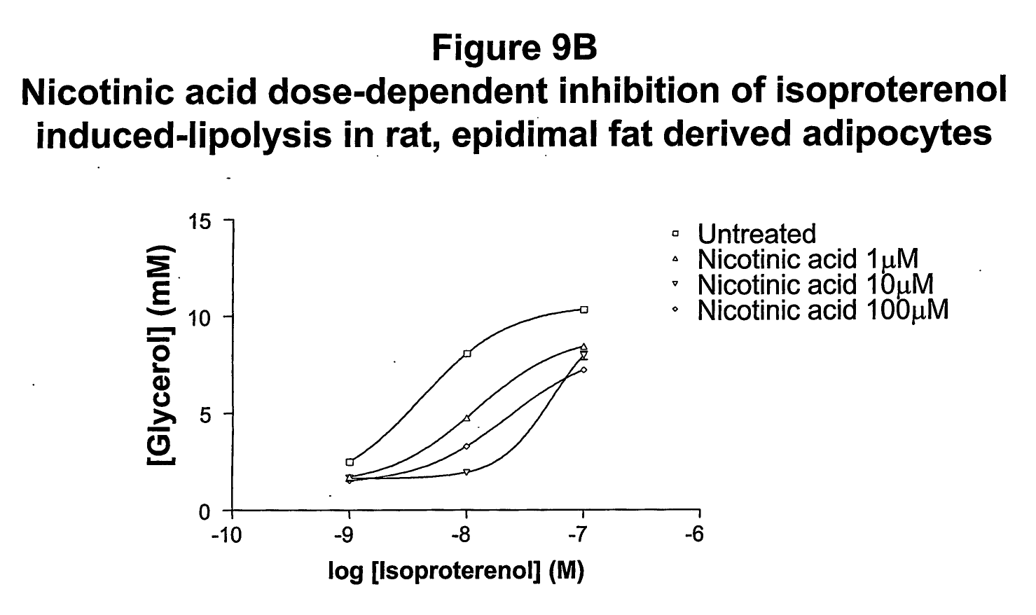

FIG. 9A depicts nicotinic acid and related compounds inhibiting isoproterenol induced lipolysis in rat epidimal fat derived adipocytes at a concentration of 10 μM. P-3-T represents 3-tetrazole-5-pyridine.

FIG. 9B illustrates a nicotinic acid dose-dependent inhibition of isoproterenol induced-lipolysis in rat epidimal fat derived adipocytes. Note the rightward shift in the dose-response curves with increasing concentrations of nicotinic acid.

Lipolysis assays were done following the isolation of adipocytes from-rat or human. The source of fat from rats was the epididymal fat and from humans was either subcutaneous or omental. Cells were isolated following collagenase digestion and floatation. An elevation of intracellular cAMP levels and concomitant activation of lipolysis via hormone sensitive lipase was accomplished using isoproterenol, forskolin, 3-isobutyl-1-methyl-xanthine (IBMX) or a combination thereof at concentrations and times determined empirically and depending on the source of tissue. Lipolysis was allowed to continue for the desired time in the presence or absence of drug (e.g. nicotinic acid, P-3-T, etc). Data was analyzed using Excel and PrismGraph.

### Example 13

Dose-Dependent Inhibition of Isoproterenol Induced-Lipolysis in Human, Subcutaneous-Derived, Primary Adipocytes via Nicotinic Acid and P-3-T

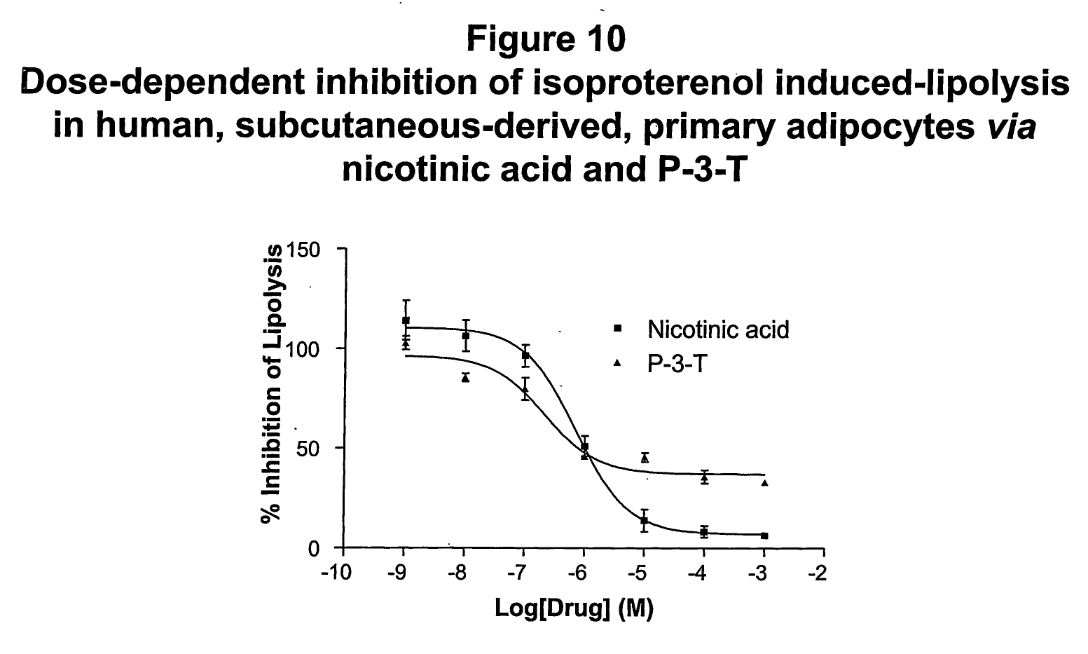

FIG. 10 illustrates the ability of both nicotinic acid and the related compound P-3-T (3-tetrazole-5-pyridine) to inhibit isoproterenol induced lipolysis in adipocyte primary cultures derived from human subcutaneous fat in a dose-dependant manner. The EC₅₀ value for nicotinic acid and P-3-T were 716 nM and 218 nM respectively.

### Example 14

SUMMARY: hRUP25, mRUP25 and rRUP25.

TABLE C

| Disclosed | | | | |
|---|---|---|---|---|
| Nicotinic | | Gi-Coupled | Shown to | |
| Acid | | (Lowers the | Inhibit | |
| Receptor | Expression by | Level of | Intra- | |
| Sub-Family | Adipocytes or | Intracellular | cellular | |
| GPCRs | Adipose | cAMP) | Lipolysis | Agonist |
| hRUP25 | yes | yes | yes | nicotinic acid; |
| | | | | nicotine; see |
| mRUP25 | yes | yes | yes | nicotinic acid |
| rRUP25 | yes | yes | yes | nicotinic acid |

### Example 15

Rodent Diabetes Models

Rodent models of type 2 diabetes associated with obesity and insulin resistance have been developed. Genetic models such as db/db and ob/ob [see Diabetes (1982) 31:1-6] in mice and fa/fa in zucker rats have been developed for understanding the pathophysiology of disease and for testing candidate therapeutic compounds [Diabetes (1983) 32:830-838; Annu Rep Sankyo Res Lab (1994) 46:1-57). The homozygous animals, C57 BL/KsJ-db/db mice developed by Jackson Laboratory are obese, hyperglycemic, hyperinsulinemic and insulin resistant [J Clin Invest (1990) 85:962-967], whereas heterozygotes are lean and normoglycemic. In the db/db model, mice progressively develop insulinopenia with age, a feature commonly observed in late stages of human type 2 diabetes when sugar levels are insufficiently controlled. Since this model resembles that of human type 2 diabetes, the compounds of the present invention are tested for activities including, but not limited to, lowering of plasma glucose and triglycerides. Zucker (fa/fa) rats are severely obese, hyperinsulinemic, and insulin resistant {Coleman, Diabetes (1982) 31:1; E Shafrir in Diabetes Mellitus, H Rifkin and D Porte, Jr, Eds [Elsevier Science Publishing Co, New York, ed. 4, (1990), pp. 299-340]}, and the fa/fa mutation can be the rat equivalent of the murine db mutation [Friedman et al, Cell (1992) 69:217-220; Truett et al, Proc Natl Acad Sci USA (1991) 88:7806]. Tubby (tub/tub) mice are characterized by obesity, moderate insulin resistance and hyperinsulinemia without significant hyperglycemia [Coleman et al, Heredity (1990) 81:424].

The present invention encompasses the use of compounds of the invention for reducing the insulin resistance and hyperglycemia in any or all of the above rodent diabetes models, in humans with type 2 diabetes or other preferred metabolic-related disorders or disorders of lipid metabolism described previously, or in models based on other mammals. Plasma glucose and insulin levels will be tested, as well as other factors including, but not limited to, plasma free fatty acids and triglycerides.

In Vivo Assay for Anti-Hyperglycemic Activity of Compounds of the Invention

Genetically altered obese diabetic mice (db/db) (male, 7-9 weeks old) are housed (7-9 mice/cage) under standard laboratory conditions at 22° C. and 50% relative humidity, and maintained on a diet of Purina rodent chow and water ad libitum. Prior to treatment, blood is collected from the tail vein of each animal and blood glucose concentrations are determined using One Touch Basic Glucose Monitor System (Lifescan). Mice that have plasma glucose levels between 250 to 500 mg/dl are used. Each treatment group consists of seven mice that are distributed so that the mean glucose levels are equivalent in each group at the start of the study. The db/db mice are dosed by micro-osmotic pumps, inserted using isoflurane anesthesia, to provide compounds of the invention, saline, or an irrelevant compound to the mice subcutaneously (s.c.). Blood is sampled from the tail vein at intervals thereafter and. analyzed for blood glucose concentrations. Significant differences between groups (comparing compounds of the invention to saline-treated) are evaluated using Student t-test.

### Example 16

Mouse Atherosclerosis Model

Adiponectin-deficient mice generated through knocking out the adiponectin gene have been shown to be predisposed to atherosclerosis and to be insulin resistant. The mice are also a suitable model for ischemic heart disease [Matsuda, M et al. J Biol Chem (2002) July, and references cited therein, the disclosures of which are incorporated herein by reference in their entirety).

Adiponectin knockout mice are housed (7-9 mice/cage) under standard laboratory conditions at 22° C. and 50% relative humidity. The mice are dosed by micro-osmotic pumps, inserted using isoflurane anesthesia, to provide compounds of the invention, saline, or an irrelevant compound to the mice subcutaneously (s.c.). Neointimal thickening and ischemic heart disease are determined for different groups of mice sacrificed at different time intervals. Significant differences between groups (comparing compounds of the invention to saline-treated) are evaluated using Student t-test.

### Example 17

In Vivo Animal Model For Dyslipidemia and Atherosclerosis

The utility of the compound of the present invention as a medical agent in the prophylaxis and treatment of a high total cholesterol/HDL-cholesterol ratio and conditions relating thereto is demonstrated by the activity of the compound in lowering the ratio of total cholesterol to HDL-cholesterol, in elevating BDL-cholesterol, or in protection from atherosclerosis in an in vivo pig model. Pigs are used as an animal model because they reflect human physiology, especially lipid metabolism, more closely than most other animal models. An illustrative in vivo pig model not intended to be limiting is presented here.

Yorkshire albino pigs (body weight 25.5±4 kg) are fed a saturated fatty acid rich and cholesterol rich (SFA-CHO) diet during 50 days (1 kg chow 35 kg⁻¹ pig weight), composed of standard chow supplemented with 2% cholesterol and 20% beef tallow [Royo T et al., European Journal of Clinical Investigation (2000) 30:843-52; which disclosure is hereby incorporated by reference in its entirety]. Saturated to unsaturated fatty acid ratio is modified from 0.6 in normal pig chow to 1.12 in the SFA-CHO diet. Animals are divided into two groups, one group (n=8) fed with the SFA-CHO diet and treated with placebo and one group (n=8) fed with the SFA-CHO diet and treated with the compound (3.0 mg kg⁻¹). Control animals are fed a standard chow for a period of 50 days. Blood samples are collected at baseline (2 days after the reception of the animals), and 50 days after the initiation of the diet. Blood lipids are analyzed. The animals are sacrificed and necropsied.

Alternatively, the foregoing analysis comprises a plurality of groups each treated with a different dose of the compound. Preferred said doses are selected from the group consisting of: 0.1 mg kg⁻¹, 0.3 mg kg⁻¹, 1.0 mg kg⁻¹, 3.0 mg kg⁻¹, 10 mg kg⁻¹, 30 mg kg⁻¹ and 100 mg kg⁻¹. Alternatively, the foregoing analysis is carried out at a plurality of timepoints. Preferred said timepoints are selected from the group consisting of 10 weeks, 20 weeks, 30 weeks, 40 weeks, and 50 weeks.

HDL-Cholesterol

Blood is collected in trisodium citrate (3.8%, 1:10). Plasma is obtained after centrifugation (1200 g 15 min) and immediately processed. Total cholesterol, HDL-cholesterol, and LDL-cholesterol are measured using the automatic analyzer Kodak Ektachem DT System (Eastman Kodak Company, Rochester, N.Y., USA). Samples with value parameters above the range are diluted with the solution supplied by the manufacturer and then re-analyzed. The total cholesterol/HDL-cholesterol ratio is determined. Comparison is made of the level of HDL-cholesterol between groups. Comparison is made of the total cholesterol/HDL-cholesterol ratio between groups.

Elevation of HDL-cholesterol or reduction of the total cholesterol/HDL-cholesterol ratio on administration of the compound is taken as indicative of the compound having the aforesaid utility.

Atherosclerosis

The thoracic and abdominal aortas are removed intact, opened longitudinally along the ventral surface, and fixed in neutral-buffered formalin after excision of samples from standard sites in the thoracic and abdominal aorta for histological examination and lipid composition and synthesis studies. After fixation, the whole aortas are stained with Sudan IV and pinned out flat, and digital images are obtained with a TV camera connected to a computerized image analysis system (Image Pro Plus; Media Cybernetics, Silver Spring, Md.) to determine the percentage of aortic surface involved with atherosclerotic lesions [Gerrity R G et al, Diabetes (2001) 50:1654-65; Cornhill J F et al, Arteriosclerosis, Thrombosis, and Vascular Biology (1985) 5:415-26; which disclosures are hereby incorporated by reference in their entirety]. Comparison is made between groups of the percentage of aortic surface involved with atherosclerotic lesions.

Reduction of the percentage of aortic surface involved with atherosclerotic lesions on administration of the compound is taken as indicative of the compound having the aforesaid utility.

### Example 18

In Vitro Biological Activity

A modified Flash Plate™ Adenylyl Cyclase kit (New England Nuclear; Cat. No. SMP004A) is used for direct identification of candidate compounds as agonists to hRUP25 in accordance with the following protocol:

Stably transfected CHO cells (clone 46) were harvested from flasks via non-enzymatic means. The cells were washed in PBS and resuspended in the manufacturer's Assay Buffer. Live cells were counted using a hemacytometer and Trypan blue exclusion, and the cell concentration was adjusted to 2×10⁶ cells/ml. cAMP standards and Detection Buffer (comprising 2 μCi of tracer [¹²⁵I]-cAMP (100 μl) to 11 ml Detection Buffer) were prepared and maintained in accordance with the manufacturer's instructions. Candidate compounds identified as per above (if frozen, thawed at room temperature) were added to their respective wells (preferably wells of a 96-well plate) at increasing concentrations (3 μl/well; 12 μM final assay concentration). To these wells, 100,000 cells in 50 μl of Assay Buffer were added and the mixture was then incubated for 30 minutes at room temperature, with gentle shaking. Following the incubation, 100 μl of Detection Buffer was added to each well, followed by incubation for 2-24 hours. Plates were counted in a Wallac MicroBeta™ plate reader using “Prot. #31” (as per manufacturer instructions).

The biological activities for several representative compounds using the above mentioned assay are shown in the table below:

| Compound No. | RUP25 (EC₅₀) (μM) |
|---|---|
| 48 | 4.3 |
| 63 | 7.9 |

The majority of the compounds of the Examples showed activities of at least about 60 μM.

### Example 19

General Synthesis of compounds of Formula (I)—Pyrazole Formation:

To a solution of NaOEt in EtOH (either prepared by the addition of Na or commereically available NaOEt; 17.4 mmol; ETOH 20 mL), is added a ketone (15 mmol) and diethyl oxalate (2.2 g, 15 mmol) at room temperature. The reaction is heated to 75° C. and maintained at the same temperature for two hours. The reaction is cooled to room temperature and treated with a solution of NH₂NH₂·HCl (1.57 g 15 mmol) in H₂O (3 mL). The subsequent reaction mixture is allowed to stir at 75° C. for two hours. The reaction is cooled to room temperature and concentrated under vacuum. The resulting residue is dissolved in 2.5M NaOH (10 mL) and heated to 95° C. After stirring for two hours, the reaction is cooled to room temperature and washed with ether (5 mL). The aqueous layer is neutralized (pH=6.5) with 2M HCl at 0° C. The reaction is stirred at 0° C. for one hour and the product is filtered. The solid is washed with H₂O (10 mL) and dried under vacuum to afford the desired compound. The product if not a solid can be purified via methods known in the art, for example, by column chromatography or HPLC.

Utilizing the above procedure with 2-hexanone (1.5 g, 15 mmol) gave the desired product, as the carboxylic acid after hydrolysis, 1.69 g (67% yield, unoptimized).

Representative Compounds of the Present Invention:

### Compound 48: 5-Methylsulfanylmethyl-2H-pyrazole-3-carboxylic acid.

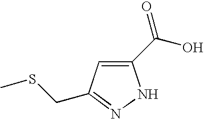

Compound 48 was prepared using a similar method as described above; LCMS: 517.4 (3M+H)⁺, 345.2 (2M+H)⁺, 173.1 (MH)⁺, 154.9, 125.1 and 107.0. NMR (400 MHz, CD₃OD, ppm): 6.62 (1H, s), 3.63 (2H, s) and 3.3 (3H, s).

### Compound 63: 5-Methoxymethyl-2H-pyrazole-3-carboxylic acid.

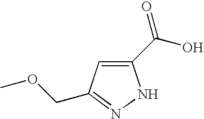

Compound 63 was prepared using a similar method as described above; LCMS: 469.3 (3M+H)⁺, 313.2 (2M+H)⁺, 157.0 (MH)⁺, 139.0, 125.1 and 107.1. NMR (400 MHz, DMSOd₆, ppm): 13.2 (1H, br. s), 6.75 (1H, s), 4.45 (2H, s) and 3.3 (3H, s).

### Compound 64: 5-(2-Ethoxy-ethyl)-2H-pyrazole-3-carboxylic acid.

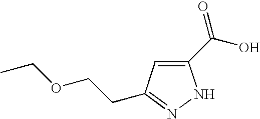

Compound 64 was prepared using a similar method as described above; LCMS: 553.5 (3M+H)⁺, 369.2 (2M+H)⁺, 185.0 (MH)⁺, 167.1 and 121.2. NMR (400 MHz, DMSOd₆, ppm): 13.1 (1H, br. s), 6.6 (1H, s), 3.68 (2H, t, J=8 Hz), 3.5 (2H, q, J=8 Hz), 2.88 (2H, t, J=8 Hz) and 1.15 (3H, t, J=8 Hz).

### Compound 65: 5-(2,2-Diethoxy-ethyl)-2H-pyrazole-3-carboxylic acid.

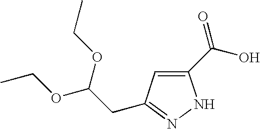

Compound 65 was prepared using a similar method as described above; LCMS: 685.5 (3M+H)⁺, 457.2 (2M+H)⁺, 229.2 (MH)⁺, 183.0 and 137.0. NMR (400 MHz, DMSOd₆, ppm): 13 (1H, br. s), 6.55 (1H, s), 4.7 (1H, t, J=4 Hz), 3.6 and 3.58 (2H, qq, J=8 Hz), 3.44 and 3.42 (2H, qq, J=8 Hz), 2.86 (2H, d, J=8 Hz) and 1.05 (6H, t, J=8 Hz).

### Example 20

General Synthesis of compounds of Formula (I)—Reductive Amination:

To a 20 mL vial with stirring bar is added 5-formyl-1H-pyrazole-3-carboxylic acid ethyl ester (1.19 mmol) and 1,2-chloroethane (3 mL). An amine (1.19 mmol) is added, followed by sodium triacetoxyborohydride (2.37 mmol). The vial is capped with a septum flushed with N₂, and stirred overnight at room temperature. The mixture is added to NaHCO₃ (10 mL) and extracted with CH₂Cl₂ (3×10 mL). The combined organic extracts are dried (Na₂SO₄), filtered, and concentrated. The crude product is purified using preparatory-BPLC or other purification method known in the art to provide compounds of Formula (I), where R₂ is not H. The purified product can be hydrolysis, for example, using a manner as described above in Example 19, to give compounds of Formula (I), where R₂ is H.

The intermediate 5-formyl-1H-pyrazole-3-carboxylic acid ethyl ester was prepared in the following manner:

Step A: Synthesis of N-2,2-Dimethoxy-ethyl)-4methyl-benzenesulfonamide.

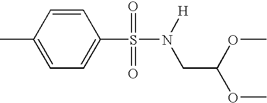

To 350 mL of dry Et₂O was added p-toluenesulfonyl chloride (73.4 g, 385 mmol). Without cooling, a mixture of 2,2-dimethoxy-ethylamine (36.8 g, 350 mmol) and triethylamine (53.7 mL, 385 mmol) was added dropwise to the first solution at a rate sufficient to give a gentle reflux. After stirring 15 h, the reaction was washed with NaHCO₃, and the organic layer was separated, dried (Na₂SO₄), filtered, and concentrated providing 95.0 g (95%) of N-(2,2-dimethoxy-ethyl)-4-methyl-benzenesulfonamide.

Step B: Synthesis of N-(2,2-dimethoxyethyl)-N-nitroso-4-tosylamide.

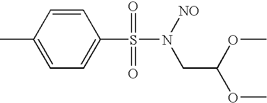

Without further purification, N-(2,2-dimethoxy-ethyl)-4-methyl-benzenesulfon-amide (95 g, 366 mmol) was taken up in dry Et₂O (350 mL) and treated with HOAc (140 mL), and Ac₂O (140 mL), cooled to 0° C. and stirred. In one portion, NaNO₂ (48.3 g, 700 mmol) was added, and the reaction was stirred 2 h at 0° C., and then 15 h at rt. Saturated NaHCO₃ (350 mL) was added, followed by solid NaNCO₃ until CO₂ bubbling stopped. The organic layer was separated, and the aqueous portion was extracted with Et₂O (4×100 mL). The combined organic layers were washed with NaHCO₃ (2×50 mL), dried (Na₂SO₄), filtered, and concentrated providing 36.4 g (34%) of N-(2,2-dimethoxyethyl)-N-nitroso-4-tosylamide.

Step C: Synthesis of 2-Diazo-1,1-dimethoxy-ethane.

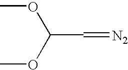

To a mixture of MeOH (140 mL), H₂O (70 mL), and Et₂O (28 mL) was added KOH (21.1 g, 377 mmol). After the KOH dissolved, the mixture was stirred vigorously at 0° C. Portionwise, N-2,2-dimethoxyethyl)-N-nitroso-4-tosylamide (36.4 g, 126 mmol) was added over 15 min., and stirred 2 h at 0° C. Ether (70 mL) and 2 M KOH (70 mL) were added, and the reaction was stirred 2 h at 0° C.; Further 2 M KOH (70 mL) was added to dissolve any solid, and the Et₂O layer was separated. The aqueous portion was extracted with Et₂O until no more yellow color was seen in the organic phase. The combined organic extracts were washed with 2 M KOH (70 mL), dried (Na₂SO₄), and filtered to provide a solution of 2diazo-1,1-dimethoxy-ethane in ether.

Step D: Synthesis of 5-Dimethoxymethyl-1H-pyrazole-3-carboxylic acid ethyl ester.

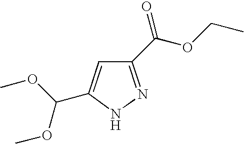

The ether solution of 2-diazo-1,1-dimethoxy-ethane in ether from the previous step was cooled to about −20° C. and treated with propynoic acid ethyl ester (12.8 mL, 126 mmol). The reaction was stirred for 2 h at 0° C., quenched with AcOH (1.8 mL), and concentrated to afford ˜27.4 g of 5-dimethoxymethyl-1H-pyrazole-3-carboxylic acid ethyl ester.

Step E: Synthesis of 5-formyl-1H-pyrazole-3-carboxylic acid ethyl ester.

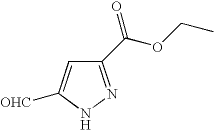

Amberlyst-15 (19 g) was added to a solution of crude 5-dimethoxymethyl-1H-pyrazole-3-carboxylic acid ethyl ester dissolved in acetone (142 mL) and H₂O (66 mL); After stirring 2 h at rt, the Amberlyst was filtered from the solution, and the filtrate was extracted with EtOAc (2×100 mL) and concentrated. The concentrate was suspended in benzene (30 mL) and hexane (120 mL), and stirred 2 h at rt. The precipitate was filtered, washed with 9:1 hexanes/benzene, and air-dried providing 5.29 g [25% from N-(2,2dimethoxyethyl)-N-nitroso-4-tosylamide] of 5-dimethoxymethyl-1H-pyrazole-3-carboxylic acid ethyl ester: LC-MS m/z 167 (M-1); ¹H NMR (400 MHz, DMSO-d₆) δ9.97 (bs, 1 H), 7.34 (bs, 1 H), 4.37 (m, 2 H), 1.36 (t, J=7.06 Hz, 3 H).

### Compound 62: 5-(Benzylamino-methyl)-1H-pyrazole-3-carboxylic acid ethyl ester.

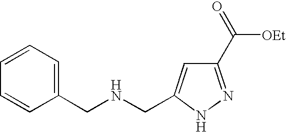

To a 20 mL vial with stirring bar was added 5-formyl-1H-pyrazole-3-carboxylic acid ethyl ester (0.20 g, 1.19 mmol) and 1,2-dichloroethane (3 mL). Benzylamine (0.130 mL, 1.19 mmol) was added, followed by sodium triacetoxyborohydride (0.504 g, 2.37 mmol). The vial was capped with a septum flushed with N₂, and stirred overnight at room temperature. The mixture was added to NaHCO₃ (10 mL) and extracted with CH₂Cl₂ (3×10 mL). The combined organic extracts were dried (Na₂SO₄), filtered, and concentrated. The crude product was purified using preparatory-HPLC providing 0.254 g (87%) of 5-(benzylamino-methyl)-1H-pyrazole-3-carboxylic acid ethyl ester: LC-MS m/z 260 (M+1); ¹H NMR (400 MHz, CDCl₃) δ9.30 (s, 1 H), 7.60-7.31 (m, 5 H), 6.87 (s, 1 H), 4.22 (q, J=7.1 Hz, 2 H), 4.11 (m, 4 H), 1.21 (t, J=7.3 Hz, 3 H).

Throughout this application, various publications, patents and published patent applications are cited. The disclosures of these publications, patents and published patent applications referenced in this application are hereby incorporated by reference in their entirety into the present disclosure. Modifications and extension of the disclosed inventions that are within the purview of the skilled artisan are encompassed within the above disclosure and the claims that follow.

Although a variety of expression vectors are available to those in the art, for purposes of utilization for both the endogenous and non-endogenous human GPCRs, it is most preferred that the vector utilized be pCMV. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCWV: ATCC #203351.

1. A compound of Formula (I): 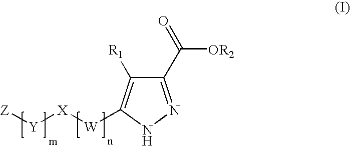
wherein:
W and Y are independently a straight or branched chain C₁₋₅ alkylene group optionally containing one double bond, one triple bond or carbonyl, wherein said C₁₋₅ alkylene group is optionally substituted with halogen, hydroxyl, C₁₋₄ alkyl, C₁₋₄ haloalkyl or C₁₋₄ alkoxy;
X is —NR₃C(O)—, —C(O)NR₃, —NR₃S(O)₂—, —S(O)₂NR₃—, —NR₃C(O)NR₄—, —NR₃C(O)O—, —OC(O)NR₃—, —NR₃—, —C(O)—, —CH(OH)—, —C(NH)—, —O—, —S—, —S(O)— or —S(O)₂—;
R₃ and R₄ are independently H, C₁₋₄ alkyl, phenyl or heteroaryl, wherein each of said alkyl, phenyl and heteroaryl are optionally substituted with 1 to 5 substituents selected from the group consisting of halogen, hydroxyl, thiol, cyano, nitro, C₁₋₄ haloalkyl, amino, C₁₋₄ alkylamino, di-C₁₋₄-alkylamino, C₁₋₄ alkyl, C₁₋₄ alkoxy, C₂₋₄ alkenyl, C₂₋₄ alkynyl, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl;
Z is H, halogen, phenyl or heteroaryl, wherein said phenyl and heteroaryl are optionally substituted with 1 to 5 substituents selected from the group consisting of halogen, hydroxy, thiol, cyano, nitro, C₁₋₄ haloalkyl, amino, C₁₋₄ alkylamino, di-C₁₋₄-alkylamino, C₁₋₄ alkyl, C₁₋₄ alkoxy, C₂₋₄ alkenyl, C₂₋₄ alkynyl, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl;
R₁ is H, hydroxyl, halogen, C₁₋₄ alkyl or C₁₋₄ haloalkyl;
R₂ is H or C₁₋₈ alkyl and
“n” and “m” are each independently 0 or 1; or
a pharmaceutically acceptable salt, solvate or hydrate thereof;
provided that:
i) when both R₁ and R₂ are H then —[W]ₙ—X—[Y]ₘ-Z together is not CO₂H, C(O)—C₆H₄-p-O—C₈H₁₇, OCH₂CH₃, OH, CH₂CH₂CH₂CH₂CO₂H, CH₂CH₂CH₂CO₂H, CH₂CO₂H and CH₂CH₂CO₂H;
ii) when R₁ is CH₃ and R₂ is H then —[W]ₙ—X—[Y]ₘ-Z together is not CH₂CO₂H, C(O)CH═CH C₆H₅, C(O)C₆H₄-p-OCH₃, CO₂H, C(O)CH₃, C(O)C₆H₄-o-CH₃, C(O)C₆H₄-o-Br, C(O)C₆H₄-o-Cl, and C(O)C₆H₅;
iii) when R₁ is Br and R₂ is H then —[W]ₙ—X—[Y]ₘ-Z together is not CO₂H;
iv) when R₁ is OH and R₂ is H then —[W]ₙ—X—[Y]ₘ-Z together is not CO₂H;
v) when R₁ is H and R₂ is CH₃ then —[W]ₙ—X—[Y]ₘ-Z together is not 2,6-dichloro-4-trifluoromethylphenoxy, C(O)NH—C₆H₄-p-OCH₂CH₃, NHC(O)CH(CH₃)₂, SCH₃, C(O)—C₆H₄-p-O-C₈H₁₇, SCH₂CH₃, C(O)NHC₆H₅, CH(OCH₃)₂, CH₂OC(O)CH₃, CO₂H, CO₂CH₃, C(O)C₆H₄-p-NO₂, C(O)C₆H₅, CH₂CH₂CO₂CH₃, CH₂CH₂CH₂CH₂CO₂CH₃, CH₂CH₂CH₂CO₂CH₃ and CH₂CO₂CH₃;
vi) when R₁ is OH and R₂ is CH₃ then —[W]ₙ—X—[Y]ₘ-Z together is not CH₂OCH₂C₆H₅, CH₂OCH(CH₃)₂ and CH₂OH;
vii) when R₂ is CH₃ then:
R₁ is not CH₃ and —[W]ₙ—X—[Y]ₘ-Z together is not 2,6-dichloro-4-trifluoromethylphenoxy;
R₁ is not I and —[W]ₙ—X—[Y]ₘ-Z together is not CO₂C(CH₃)₃;
R₁ is not C(CH₃)₃ and —[W]ₙ—X—[Y]ₘ-Z together is not formyl;
R₁ is not Br and —[W]ₙ—X—[Y]ₘ-Z together is not CO₂CH₃; and
R₁ is not CH₂CH₂CH₂CH₃ and —[W]ₙ—X—[Y]ₘ-Z together is not formyl;
viii) when R₁ is H and R₂ is CH₂CH₃ then —[W]ₙ—X—[Y]ₘ-Z together is not CH₂SCH₂CH₃, OCH₂CH₂CH═CH₂, CH₂CH₂CH₂OH, CH₂CH₂CHO, CO₂CH₂CH₃, OCH₃, C(O)CH₂Br, CO₂C₈H₁₇, formyl, OH, CH₂N(CH₂CH₂Cl)₂, CH(CH₃)OC(O)CH₃, CH₂OH, CH₂OC(O)CH₃, C(O)CH₃, C(O)C₆H₅ and C(O)NHCH₂CO₂CH₂CH₃.
ix) when R₁ is CH₃ and R₂ is CH₂CH₃ then —[W]ₙ—X—[Y]ₘ-Z together is not CH(OH)C₆H₄-p-N(CH₃)₂, C(O)CH₂C(O)CH₃, CO₂CH₂C₆H₅, CO₂CH₃, C(O)CH₂CH₂CH₃, C(O)CH₃, C(O)C₆H₄-p-OCH₃, C(O)C₆H₄-o-Br, C(O)C₆H₄-p-Cl, C(O)C₆H₄-o-Cl, C(O)CH₂C₆H₅ and C(O)C₆H₅;
x) when R₂ is CH₂CH₃ then:
R₁ is not I and —[W]ₙ—X—[Y]ₘ-Z together is not CO₂CH₂CH₃;
R₁ is not CF₃ and —[W]ₙ—X—[Y]ₘ-Z together is not CO₂CH₂CH₃; and
R₁ is not Br and —[W]ₙ—X—[Y]ₘ-Z together is not CO₂CH₂CH₃;
xi) when R₁ is OH and R₂ is CH₂CH₃ then −[W]ₙ—X—[Y]ₘ-Z together is not C(O)C₆H₅, C(O)NH₂ and CO₂CH₂CH₃;
xii) when R₁ is H and R₂ is C(CH₃)₃ then —[W]ₙ—X—[Y]ₘ-Z together is not CO₂C(CH₃)₃, C(O)NHC(O)CH₃ and C(O)NH₂;
xiii) when R₁ is OH and R₂ is CH₂CH₂CH₂CH₃ then —[W]ₙ—X—[Y]ₘ-Z together is not C(O)C₆H₅; and
xiv) when X is —NR₃— then “n” is 1.

2. The compound according to claim 1 wherein “n” is 0.

3. The compound according to claim 1 wherein “n” is 1.

4-149. (canceled)

150. The compound according to claim 1 wherein “m” is 0.

151. The compound according to claim 1 wherein “m” is 1.

152. The compound according to claim 1 wherein W is the straight or branched C₁₋₅ alkylene group optionally containing one double bond, one triple bond or carbonyl, wherein said C₁₋₅ alkylene group is optionally substituted with halogen, hydroxyl, C₁₋₄ alkyl or C₁₋₄ alkoxy.

153. The compound according to claim 152 wherein W is selected from the group consisting of —CH₂—, —CH₂CH₂—, —CH(CH₃)CH₂—, —CH₂CH(CH₃)—, —C(CH₃)₂CH₂—, —CH₂C(CH₃)₂—, —CH₂CH₂CH₂—, —CH₂CH₂CH₂CH₂—, —CH₂C(O)—, —C(O)CH₂—, —CH(CH₃)C(O)—, —C(O)CH(CH₃)—, —CH₂CH₂C(O)—, —C(O)CH₂CH₂—, —C(CH₃)₂C(O)—, —C(O)C(CH₃)₂—, —C(CH₃)₂CH₂C(O)—, —C(O)CH₂C(CH₃)₂—, —CH₂C(O)CH₂—, —CH₂CH₂CH₂C(O)—, —C(O)CH₂CH₂CH₂—, —CH(CH₃)CH₂CH₂C(O)—, —C(O)CH₂CH₂CH(CH₃)—, —CH₂CH₂C(O)CH₂—, —CH₂C(O)CH₂CH₂—, —CH═CHC(O)—, —C(O)CH═CH—, —C(CH₃)═CHC(O)—, and —C(O)CH═C(CH₃)—, each optionally substituted with halogen, hydroxyl, C₁₋₄ alkyl or C₁₋₄ alkoxy.

154. The compound according to claim 152 wherein W is —CH(CH₃)—, —CH(OCH₃)CH₂—, or —CH₂CH(OCH₃)—, each optionally substituted with halogen, hydroxyl, C₁₋₄ alkyl or C₁₋₄ alkoxy.

155. The compound according to claim 152 wherein W is selected from the group consisting of —CH₂—, —CH(CH₃)—, —C(CH₃)₂—, —CH₂CH₂—, —CH(CH₃)CH₂—, —CH₂CH(CH₃)—, —C(CH₃)₂CH₂—, —CH₂C(CH₃)₂—, —CH(OCH₃)CH₂—, —CH₂CH(OCH₃)—, —CH₂CH₂CH₂—, —CH₂CH₂CH₂CH₂—, —CH₂C(O)—, —C(O)CH₂—, —CH(CH₃)C(O)—, —C(O)CH(CH₃)—, —CH₂CH₂C(O)—, —C(O)CH₂CH₂—, —C(CH₃)₂C(O)—, —C(O)C(CH₃)₂—, —C(CH₃)₂CH₂C(O)—, —C(O)CH₂C(CH₃)₂—, —CH₂C(O)CH₂—, —CH₂CH₂CH₂C(O)—, —C(O)CH₂CH₂CH₂—, —CH(CH₃)CH₂CH₂C(O)—, —C(O)CH₂CH₂CH(CH₃)—, —CH₂CH₂C(O)CH₂—, —CH₂C(O)CH₂CH₂—, —CH═CHC(O)—, —C(O)CH═CH—, —C(CH₃)═CHC(O)—, and —C(O)CH═C(CH₃)—.

156. The compound according to claim 152 wherein W is —CH═CH—, —C≡C—, or —C(O)—.

157. The compound according to claim 1 wherein Y is the straight or branched chain C₁₋₅ alkylene group optionally containing one double bond, one triple bond or carbonyl, wherein said C₁₋₅ alkylene group is optionally substituted with halogen, hydroxyl, C₁₋₄ alkyl or C₁₋₄ alkoxy.

158. The compound according to claim 157 wherein Y is selected from the group consisting of —CH₂—, —CH₂CH₂—, —CH(CH₃)CH₂—, —CH₂CH(CH₃)—,—C(CH₃)₂CH₂—, —CH₂C(CH₃)₂—, —CH₂CH₂CH₂—, —CH₂CH₂CH₂CH₂—, —C≡CCH₂—, —CH₂C≡C—, —CH₂C(O)—, —C(O)CH₂—, —CH(CH₃)C(O)—, —C(O)CH(CH₃)—, —CH₂CH₂C(O)—, —C(O)CH₂CH₂—, —C(CH₃)₂CH₂C(O)—, —C(O)CH₂C(CH₃)₂—, —CH₂C(O)CH₂—, —CH₂CH₂CH₂C(O)—, —C(O)CH₂CH₂CH₂—, —CH(CH₃)CH₂CH₂C(O)—, —C(O)CH₂CH₂CH(CH₃)—, —CH₂CH₂C(O)CH₂—, —CH₂C(O)CH₂CH₂—, —CH═CHC(O)—, —C(O)CH═CH—, —C(CH₃)═CHC(O)—, and —C(O)CH═C(CH₃)—, each optionally substituted with halogen, hydroxyl, C₁₋₄ alkyl or C₁₋₄ alkoxy.

159. The compound according to claim 157 wherein Y is selected from the group consisting of —CH₂—, —CH₂CH₂—, —CH(CH₃)CH₂—, —CH₂CH(CH₃)—,—C(CH₃)₂CH₂—, —CH₂C(CH₃)₂—, —CH₂CH₂CH₂—, —CH₂CH₂CH₂CH₂—, —C≡CCH₂—, —CH₂C≡C—, —CH₂C(O)—, —C(O)CH₂—, —CH(CH₃)C(O)—, —C(O)CH(CH₃)—, —CH₂CH₂C(O)—, —C(O)CH₂CH₂—, —C(CH₃)₂CH₂C(O)—, —C(O)CH₂C(CH₃)₂—, —CH₂C(O)CH₂—, —CH₂CH₂CH₂C(O)—, —C(O)CH₂CH₂CH₂—, —CH(CH₃)CH₂CH₂C(O)—, —C(O)CH₂CH₂CH(CH₃)—, —CH₂CH₂C(O)CH₂—, —CH₂C(O)CH₂CH₂—, —CH═CHC(O)—, —C(O)CH═CH—, —C(CH₃)═CHC(O)—, and —C(O)CH═C(CH₃)—.

160. The compound according to claim 157 wherein Y is —CH(CH₃)— optionally substituted with halogen, hydroxyl or C₁₋₄ alkoxy.

161. The compound according to claim 157 wherein Y is —CH(OCH₃)CH₂— or —CH₂CH(OCH₃)— optionally substituted with halogen, hydroxyl or C₁₋₄ alkyl.

162. The compound according to claim 157 wherein Y is —CH═CH— optionally substituted with C₁₋₄ alkyl or C₁₋₄ alkoxy.

163. The compound according to claim 157 wherein Y is —C(CH₃)₂—, —C≡C—, —C(O)—, —C(CH₃)₂C(O)—, or —C(O)C(CH₃)₂—.

164. The compound according to claim 1 wherein X is —NHC(O)— or —C(O)NH—.

165. The compound according to claim 1 wherein X is —NH— or —NCH₃—.

166. The compound according to claim 1 wherein X is selected from the group consisting of —C(O)—, —CH(OH)—, —C(NH)—, —O—, —S—, —S(O)—, or —S(O)₂—.

167. The compound according to claim 1 wherein Z is H, halogen, or phenyl.

168. The compound according to claim 1 wherein Z is phenyl optionally substituted with 1 to 3 substituents selected from the group consisting of —F, —Cl, —Br, —CF₃, —NHCH₃, —N(CH₃)₂, —CH₃, —CH₂CH₃, —OCH₃ and —OCF₃.

169. The compound according to claim 1 wherein Z is heteroaryl optionally substituted with 1 to 3 substituents selected from the group consisting of —F, —Cl, —Br, —CF₃, —NHCH₃, —N(CH₃)₂, —CH₃, —CH₂CH₃, —OCH₃ and —OCF₃.

170. The compound according to claim 1 wherein R₁ is H.

171. The compound according to claim 1 wherein R₁ is hydroxyl.

172. The compound according to claim 1 wherein R₁ is halogen.

173. The compound according to claim 1 wherein R₁ is C₁₋₄ alkyl.

174. The compound according to claim 1 wherein R₁ is C₁₋₄ haloalkyl.

175. The compound according to claim 1 wherein R₂ is H.

176. The compound according to claim 1 wherein R₂ is C₁₋₈ alkyl.

177. The compound according to claim 1 selected from the group consisting of:
5-Ethylsulfanylmethyl-1H-pyrazole-3-carboxylic acid;
5-Ethanesulfmylmethyl-1H-pyrazole-3-carboxylic acid;
5-Ethanesulfonylmethyl-1H-pyrazole-3-carboxylic acid;
5-(2-Oxo-propoxymethyl)-1H-pyrazole-3-carboxylic acid;
5-Prop-2-ynyloxymethyl-1H-pyrazole-3-carboxylic acid;
5-Carbamoyl-1H-pyrazole-3-carboxylic acid;
5-(1-Methylsulfanyl-ethyl)-1H-pyrazole-3-carboxylic acid;
5-(1-Methanesulfinyl-ethyl)-1H-pyrazole-3-carboxylic acid;
5-(1-Methanesulfonyl-ethyl)-1H-pyrazole-3-carboxylic acid;
5-(1,1-Dimethoxy-ethyl)-1H-pyrazole-3-carboxylic acid;
5-(2-Carboxy-1,1-dimethyl-ethyl)-1H-pyrazole-3-carboxylic acid;
5-(1-Acetoxy-ethyl)-1H-pyrazole-3-carboxylic acid;
5-(3-Hydroxy-propyl)-1H-pyrazole-3-carboxylic acid;
5-(1-Chloro-3-hydroxy-propyl)-1H-pyrazole-3-carboxylic acid;
5-(2-Hydroxy-ethyl)-1H-pyrazole-3-carboxylic acid;
5-(2-Hydroxy-1-methyl-ethyl)-1H-pyrazole-3-carboxylic acid;
5-(2-Carboxy-1-methyl-vinyl)-1H-pyrazole-3-carboxylic acid;
5-Propylcarbamoylmethyl-1H-pyrazole-3-carboxylic acid;
5-(2-Amino-vinyl)-1H-pyrazole-3-carboxylic acid;
5-(2-Amino-propyl)-1H-pyrazole-3-carboxylic acid;
5-(2-Dimethylamino-1-methyl-ethyl)-1H-pyrazole-3-carboxylic acid;
5-(1-Hydroxy-ethyl)-1H-pyrazole-3-carboxylic acid;
5-(1-Hydroxy-1-methyl-ethyl)-1H-pyrazole-3-carboxylic acid
5-(2-Hydroxy-2-methyl-propyl)-1H-pyrazole-3-carboxylic acid;
5-(3-Carboxy-1-methyl-propyl)-1H-pyrazole-3-carboxylic acid;
5-(2-Carboxy-vinyl)-1H-pyrazole-3-carboxylic acid;
5-(2-Methoxy-vinyl)-1H-pyrazole-3-carboxylic acid;
5-(3-Acetoxy-propyl)-1H-pyrazole-3-carboxylic acid;
5-Carbamoylmethyl-1H-pyrazole-3-carboxylic acid;
5-Hydroxymethyl-1H-pyrazole-3-carboxylic acid;
5-(2,2-Dimethoxy-ethyl)-1H-pyrazole-3-carboxylic acid;
5-(2-Imino-propyl)-1H-pyrazole-3-carboxylic acid;
5-(2-Amino-2-methyl-propyl)-1H-pyrazole-3-carboxylic acid;
5-(Ethoxycarbonyl-fluoro-methyl)-1H-pyrazole-3-carboxylic acid;
5-(1-Ethoxycarbonyl-ethyl)-1H-pyrazole-3-carboxylic acid;
5-Ethoxycarbonylmethyl-1H-pyrazole-3-carboxylic acid;
5-(2-Ethoxycarbonyl-ethyl)-1H-pyrazole-3-carboxylic acid;
5-Methoxymethyl-1H-pyrazole-3-carboxylic acid;
5-(1-Methoxycarbonyl-1methyl-ethyl)-1H-pyrazole-3-carboxylic acid;
5-(1-Hydroxy-1-methoxycarbonyl-ethyl)-1H-pyrazole-3-carboxylic acid;
5-(3-Methoxycarbonyl-propyl)-1H-pyrazole-3-carboxylic acid;
5-2-Methoxycarbonyl-vinyl)-1H-pyrazole-3-carboxylic acid;
5-Dimethylcarbamoylmethyl-1H-pyrazole-3-carboxylic acid;
1H-Pyrzole-3,5-dicarboxylic acid;
5-Ethoxymethyl-1H-pyrazole-3-carboxylic acid;
5-(2-Methoxy-ethyl)-1H-pyrazole-3-carboxylic acid;
5-(3-Methoxy-propyl)-1H-pyrazole-3-carboxylic acid;
5-Methylsulfanylmethyl-1H-pyrazole-3-carboxylic acid;
5-Methanesulfinylmethyl-1H-pyrazole-3-carboxylic acid;
5-Methanesulfonylmethyl-1H-pyrazole-3-carboxylic acid;
5-(2-Methylsulfanyl-ethyl)-1H-pyrazole-3-carboxylic acid;
5-(2-Methanesulfinyl-ethyl)-1H-pyrazole-3-carboxylic acid;
5-(2-Methanesulfonyl-ethyl)-1H-pyrazole-3-carboxylic acid;
5-(3-Methylsulfanyl-propyl)-1H-pyrazole-3-carboxylic acid;
5-(3-Methanesulfinyl-propyl)-1H-pyrazole-3-carboxylic acid;
5-(3-Methanesulfonyl-propyl)-1H-pyrazole-3-carboxylic acid;
5-(2-Amino-ethyl)-1H-pyrazole-3-carboxylic acid;
5-(2-Methylamino-ethyl)-1H-pyrazole-3-carboxylic acid;
5-(2-Dimethylamino-ethyl)-1H-pyrazole-3-carboxylic acid;
5-(2-Oxo-propyl)-1H-pyrazole-3-carboxylic acid;
5-(3-Oxo-butyl)-1H-pyrazole-3-carboxylic acid;
5-(Benzylamino-methyl)-1H-pyrazole-3-carboxylic acid;
5-Methoxymethyl-1H-pyrazole-3-carboxylic acid;
5-Ethoxymethyl-1H-pyrazole-3-carboxylic acid; and
5-(2,2-Diethoxy-ethyl)-1H-pyrazole-3-carboxylic acid; or
a pharmaceutically acceptable salt, solvate or hydrate thereof.

178. A pharmaceutical composition comprising a pharmaceutically acceptable carrier in combination with at least one compound according to Formula (I): 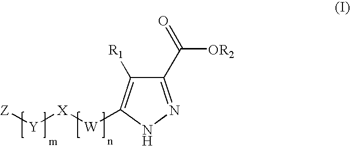
wherein:
W and Y are independently a straight or branched chain C₁₋₅ alkylene group optionally containing one double bond, one triple bond or carbonyl, wherein said C₁₋₅ alkylene group is optionally substituted with halogen, hydroxyl, C₁₋₄ alkyl, C₁₋₄ haloalkyl or C₁₋₄ alkoxy;
X is —NR₃C(O)—, —C(O)NR₃, —NR₃S(O)₂—, —S(O)₂NR₃—, —NR₃C(O)NR₄—, —NR₃C(O)O—, —OC(O)NR₃—, —NR₃—, —C(O)—, —CH(OH)—, —C(NH)—, —O—, —S—, —S(O)— or —S(O)₂—;
R₃ and R₄ are independently H, C₁₋₄ alkyl, phenyl or heteroaryl, wherein each of said alkyl, phenyl and heteroaryl are optionally substituted with 1 to 5 substituents selected from the group consisting of halogen, hydroxyl, thiol, cyano, nitro, C₁₋₄ haloalkyl, amino, C₁₋₄ alkylamino, di-C₁₋₄-alkylamino, C₁₋₄ alkyl, C₁₋₄ alkoxy, C₂₋₄ alkenyl, C₂₋₄ alkynyl, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl;
Z is H, halogen, phenyl or heteroaryl, wherein said phenyl and heteroaryl are optionally substituted with 1 to 5 substituents selected from the group consisting of halogen, hydroxy, thiol, cyano, nitro, C₁₋₄ haloalkyl, amino, C₁₋₄ alkylamino, di-C₁₋₄-alkylamino, C₁₋₄ alkyl, C₁₋₄ alkoxy, C₂₋₄ alkenyl, C₂₋₄ alkynyl, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl and C₁₋₄ haloalkylsulfonyl;
R₁ is H, hydroxyl, halogen, C₁₋₄ alkyl or C₁₋₄ haloalkyl;
R₂ is H or C₁₋₈ alkyl and
“n” and “m” are each independently 0 or 1; or
a pharmaceutically acceptable salt, solvate or hydrate thereof;
provided that when X is —NR₃— then “n” is 1.

179. A method for prophylaxis or treatment of a metabolic-related disorder in an individual in need of said prophylaxis or treatment comprising administering to the individual a therapeutically effective amount of a compound according to claim 1 or a pharmaceutical composition according to claim 178.

180. The method according to claim 179 wherein the metabolic-related disorder is selected from the group consisting of dyslipidemia, atherosclerosis, coronary heart disease, insulin resistance, obesity, impaired glucose tolerance, atheromatous disease, hypertension, stroke, Syndrome X, heart disease and type 2 diabetes.

181. The method according to claim 180 wherein the metabolic-related disorder is dyslipidemia, atherosclerosis, coronary heart disease, insulin resistance and type 2 diabetes.

182. The method according to claim 180 wherein the metabolic-related disorder is dyslipidemia.

183. The method according to claim 180 wherein the metabolic-related disorder is atherosclerosis.

184. The method according to claim 180 wherein the metabolic-related disorder is coronary heart disease.

185. The method according to claim 180 wherein the metabolic-related disorder is insulin resistance.

186. The method according to claim 180 wherein the metabolic-related disorder is type 2 diabetes.

187. The method of producing a pharmaceutical composition comprising admixing at least one compound according to claim 1 and a pharmaceutically acceptable carrier or excipient.


SEQUENCE LISTING (4 sequences)

SEQ ID NO: 1 - DNA, 22 bp, Artificial
  Novel Sequence
gctggagcattcactaggcgag

SEQ ID NO: 2 - DNA, 24 bp, Artificial
  Novel Sequence
agatcctggttcttggtgacaatg

SEQ ID NO: 3 - DNA, 27 bp, Artificial
  Novel Sequence
ctgatggacaactatgtgaggcgttgg

SEQ ID NO: 4 - DNA, 27 bp, Artificial
  Novel Sequence
gctgaagctgctgcacaaatttgcacc